(19) United States
(12) Patent Application Publication
(10) Pub. No.: US 2026/0001830 A1
(43) Pub. Date: Jan. 1, 2026

(54) PROCESS FOR PD(II)-CATALYZED SITE-SELECTIVE BETA- AND GAMMA-C(SP3)-H ARYLATION OF PRIMARY ALDEHYDES CONTROLLED BY TRANSIENT DIRECTING GROUPS

(71) Applicant: The Scripps Research Institute, La Jolla, CA (US)

(72) Inventors: Jin-Quan Yu, San Diego, CA (US); Yi-Hao Li, San Diego, CA (US)

(21) Appl. No.: 18/834,084

(22) PCT Filed: Jan. 30, 2023
(86) PCT No.: PCT/US2023/061574

§ 371 Date: Jul. 29, 2024

Related U.S. Application Data
(60) Provisional application No. 63/304,930, filed on Jan. 31, 2022.

(51) Int. Cl. C07C 45/68; C07C 51/353; C07C 67/343; C07C 201/12; C07C 253/30; C07D 221/14; C07J 1/00

(52) U.S. Cl. CPC C07C 45/68; C07C 51/353; C07C 67/343; C07C 201/12; C07C 253/30; C07D 221/14; C07J 1/00; C07C 2601/08; C07C 2601/14; C07C 2602/20

(57) ABSTRACT

The present invention provides a process for Pd(II)-catalyzed site selective β- and γC(sp3)-II arylation of primary aldehydes controlled by transient directing groups.

### CROSS REFERENCE TO RELATED APPLICATION

This application claims priority to U.S. provisional patent application No. 63/304,930, which was filed on Jan. 31, 2022, and which is hereby incorporated by reference in its entirety.

### GOVERNMENTAL SUPPORT

This invention was made with government support under grant number NIGMS R01 GM084019 awarded by the Institutes of Health (NIH). The government has certain rights in the invention.

### FIELD OF INVENTION

This invention provides a process for PdII-catalyzed site selective β- and γ-C(sp3)—H arylation of primary aldehydes controlled by transient directing groups.

### BACKGROUND OF THE DISCLOSURE

L,X-type transient directing groups (TDG) have emerged as a powerful tool in Pd(II)-catalyzed C—H functionalization since the first report in 2016.¹ Without the need of directing group installation and removal, the discovery and development of this class of TDG represents a significant advance for directed C—H activation reactions of ketones, aldehydes and amines that can form reversible imines with TDG.^{2,3} Compared to ketone and amine substrates, C—H functionalizations of aldehydes are underdeveloped. Our initial report using glycine as a TDG for the C(sp³)—H arylation was limited to ketones or o-tolualdehydes.¹ Subsequently, other benzylic and ortho-functionalizations of benzaldehyde derivatives have been extensively investigated.^{4.5} Amino acid-based TDG has also been developed for aliphatic aldehydes by the Ge group and others to achieve β- and γ-arylation (Scheme 1a).⁶ Despite these advances, substrates were limited to secondary or tertiary aldehydes. For primary aldehydes, only two individual examples have been reported to date by Li and Ge with 46% and 25% yield for methyl and methylene C(sp³)—H arylation, respectively.^{6a} In addition, β-methylene C—H functionalization of acyclic primary aldehydes remains to be developed.^{6a,c,g} Most importantly, controlling site-selectivity in C(sp³)—H activation by designing different TDG has not been demonstrated thus far. Notably, an alternative approach for B-arylation of aliphatic aldehydes have also been pursued via a radical pathway using cyanobenzenes and excess amount of aldehydes (Scheme 1b).⁷

There is a need for methodology for efficient β- and γ-arylation of primary aldehydes.

### SUMMARY OF THE DISCLOSURE

Some embodiments described herein provide a process for preparing a compound of Formula (I)

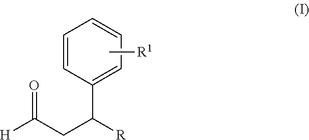

wherein:
R is C₁-C₁₀ alkyl that is optionally substituted with one substituent selected from the group consisting of C₃-C₇ cycloalkyl, C₆-C₁₀ aryl, halo, —O—C(═O)-(C₁-C₆ alkyl), —O-(C₆-C₁₀ aryl), and —O—(C₁-C₆ alkyl) wherein the C₁-C₆ alkyl of the —O-(C₁-C₆ alkyl) is optionally substituted with C₆-C₁₀ aryl; C₃-C₇cycloalkyl; and C₆-C₁₀ aryl;
R¹ is hydrogen, halo,-C (=O)-(C₁-C₆ alkyl), halo (C₁-C₆ alkyl), C₁-C₆ alkyl, —NO₂, —C(═O)—OH, —CN, —C(═O)—O-(C₁-C₆ alkyl), and —O-(C₁-C₆ alkyl), or —O-halo(C₁-C₆ alkyl); comprising reacting a compound of Formula (II)

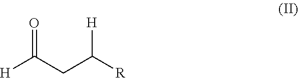

wherein R in Formula (II) is as defined above for Formula (I);
with a compound of Formula (III)

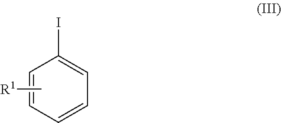

wherein R¹ in Formula (III) is as defined above for Formula (I);
in the presence of a palladium salt, an amino acid based transient directing group compound (TDG), a 2-pyridone ligand (L), a salt composition comprising at least one silver salt, and an acid composition comprising acetic acid or an acetic acid derivative.

Some embodiments described herein provide a process for preparing a compound of Formula (IV)

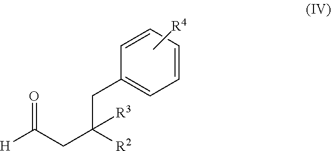

wherein:
R² is H, or C₁-C₆ alkyl;
R³ is C₃-C₇ cycloalkyl; or C₁-C₆ alkyl which is optionally substituted with a substituent selected from the group consisting of —O—C(—O)-C₁-C₆ alkyl and C₆-C₁₀ aryl; and
R⁴ is selected from the group consisting of —C(═O)-(C₁-C₆ alkyl); —C(═O)—O-(C₁-C₆ alkyl); —C(═O)—O-(C₃-C₁₀ bicyclic carbocyclyl) wherein said C₃-C₁₀ bicyclic carbocyclyl is optionally substituted with C₁-C₆ alkyl; halo; halo(C₁-C₆ alkyl); —NO₂; —CN;

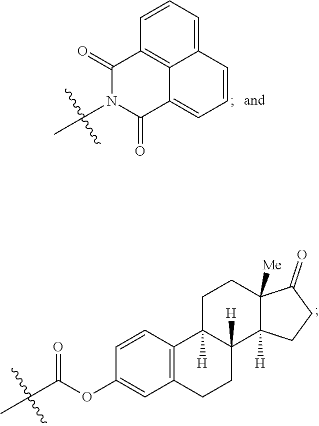

comprising reacting a compound of Formula (V)

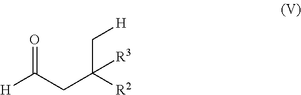

wherein R² and R³ in Formula (V) are as defined above for Formula (IV);
with a compound of Formula (VI)

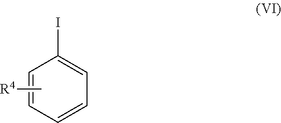

wherein R⁴ in Formula (VI) is as defined above for Formula (IV);

in the presence of a palladium salt, an amino acid based transient directing group compound (TDG), a 2-pyridone ligand (L), a salt composition comprising at least one silver salt, and an acid composition comprising acetic acid or an acetic acid derivative.

Some embodiments described herein also provide a process for preparing a compound of Formula (VII)

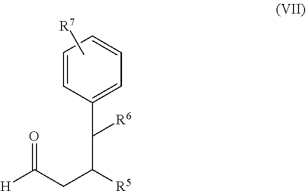

wherein:
R⁵ and R⁶ together with the carbon atoms to which they are shown attached form a C₅-C₇ cycloalkyl; and
R⁷ is selected from the group consisting of —C(—O)-(C₁-C₆ alkyl); —C(═O)—O-(C₁-C₆ alkyl); —C(═O)—O-(C₃-C₁₀ bicyclic carbocyclyl) wherein said C₃-C₁₀ bicyclic carbocyclyl is optionally substituted with C₁-C₆ alkyl; halo; halo (C₁-C₆ alkyl); —NO₂; —CN;

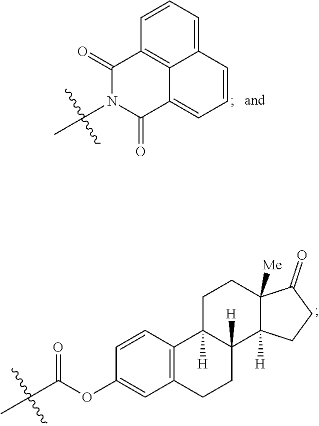

comprising reacting a compound of Formula (VIII)

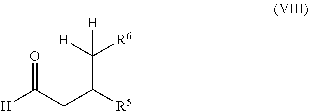

wherein R⁵ and R⁶ are as defined above for Formula (VII);
with a compound of Formula (IX)

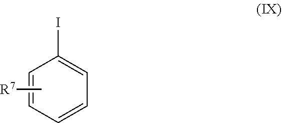

wherein R⁷ is as defined above for Formula (VII);
in the presence of a palladium salt, an amino acid based transient directing group compound (TDG), a 2-pyridone ligand (L), a salt composition comprising at least one silver salt, and an acid composition comprising acetic acid or an acetic acid derivative.

### BRIEF DESCRIPTION OF THE FIGURES

FIG. 1 shows the kinetic isotope effect (KIE) for β-C—H arylation: k_{H}/k_{D}=7.8

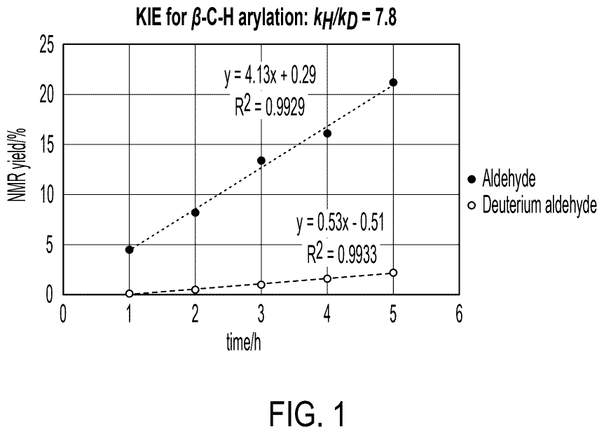

FIG. 2 shows the kinetic isotope effect (KIE) for γ-C—H arylation: k_{H}/k_{D}=5.6

FIG. 3 shows a plot of relative quasi-harmonic Gibbs free energies (Δqh-G₃₈₃) in kcal/mol for the C(sp³)—H cleavage TS in the analyzed ensembles for structures within 5 kcal/mol (corresponding to >99.9% of Boltzmann population).

### DETAILED DESCRIPTION OF THE DISCLOSURE

Unless otherwise stated, the following terms used in the specification and claims are defined for the purposes of this Application and have the following meaning. All undefined technical and scientific terms used in this Application have the meaning as commonly understood by one of ordinary skill in the art to which this disclosure belongs.

As used herein, “a” or “an” entity refers to one or more of that entity; for example, a compound refers to one or more compounds or at least one compound unless stated otherwise. As such, the terms “a” (or “an”), “one or more”, and “at least one” can be used interchangeably herein.

When a range of values is listed, it is intended to encompass each value and sub-range within the range. For example, “C₁₋₆ alkyl” is intended to encompass, C₁, C₂, C₃, C₄, C₅, C₆, C₁₋₆, C₁₋₅, C₁₋₄, C₁₋₃, C₁₋₂, C₂₋₆, C₂₋₅, C₂₋₄, C₂₋₃, C₃₋₆, C₃₋₅, C₃₋₄, C₄₋₆, C₄₋₅, and C₅₋₆ alkyl.

“Alkyl” refers to a radical of a straight-chain or branched saturated hydrocarbon group having from 1 to 20 carbon atoms (“C₁₋₂₀ alkyl”). In some embodiments, an alkyl group has 1 to 15 carbon atoms (“C₁₋₁₅ alkyl”). In some embodiments, an alkyl group has 1 to 14 carbon atoms (“C₁₋₁₄ alkyl”). In some embodiments, an alkyl group has 1 to 13 carbon atoms (“C₁₋₁₃ alkyl”). In some embodiments, an alkyl group has 1 to 12 carbon atoms (“C₁₋₁₂ alkyl”). In some embodiments, an alkyl group has 1 to 11 carbon atoms (“C₁₋₁₁ alkyl”). In some embodiments, an alkyl group has 1 to 10 carbon atoms (“C₁₋₁₀ alkyl”). In some embodiments, an alkyl group has 1 to 9 carbon atoms (“C₁₋₉ alkyl”). In some embodiments, an alkyl group has 1 to 8 carbon atoms (“C₁₋₈ alkyl”). In some embodiments, an alkyl group has 1 to 7 carbon atoms (“C₁₋₇ alkyl”). In some embodiments, an alkyl group has 1 to 6 carbon atoms (“C₁₋₆ alkyl”). In some embodiments, an alkyl group has 1 to 5 carbon atoms (“C₁₋₅ alkyl”). In some embodiments, an alkyl group has 1 to 4 carbon atoms (“C₁₋₄ alkyl”). In some embodiments, an alkyl group has 1 to 3 carbon atoms (“C₁₋₃ alkyl”). In some embodiments, an alkyl group has 1 to 2 carbon atoms (“C₁₋₂ alkyl”). In some embodiments, an alkyl group has 1 carbon atom (“C₁ alkyl”). In some embodiments, an alkyl group has 2 to 6 carbon atoms (“C₂₋₆ alkyl”). Examples of C₁₋₆ alkyl groups include methyl (C₁), ethyl (C₂), n-propyl (C₃), isopropyl (C₃), n-butyl (C₄), tert-butyl (C₄), sec-butyl (C₄), iso-butyl (C₄), n-pentyl (C₅), 3-pentanyl (C₅), amyl (C₅), neopentyl (C₅), 3-methyl-2-butanyl (C₅), tertiary amyl (C₅), and n-hexyl (C₆). Additional examples of alkyl groups include n-heptyl (C₇), n-octyl (C₈) and the like.

“Alkenyl” refers to a radical of a straight-chain or branched hydrocarbon group having from 2 to 10 carbon atoms and 1, 2, 3, or 4 carbon-carbon double bonds (“C₂₋₁₀ alkenyl”). In some embodiments, an alkenyl group has 2 to 9 carbon atoms (“C₂₋₉ alkenyl”). In some embodiments, an alkenyl group has 2 to 8 carbon atoms (“C₂₋₈ alkenyl”). In some embodiments, an alkenyl group has 2 to 7 carbon atoms (“C₂₋₇ alkenyl”). In some embodiments, an alkenyl group has 2 to 6 carbon atoms (“C₂₋₆ alkenyl”). In some embodiments, an alkenyl group has 2 to 5 carbon atoms (“C₂₋₅ alkenyl”). In some embodiments, an alkenyl group has 2 to 4 carbon atoms (“C₂₋₄ alkenyl”). In some embodiments, an alkenyl group has 2 to 3 carbon atoms (“C₂₋₃ alkenyl”). In some embodiments, an alkenyl group has 2 carbon atoms (“C₂ alkenyl”). The one or more carbon-carbon double bonds can be internal (such as in 2-butenyl) or terminal (such as in 1-butenyl). Examples of C₂₋₄ alkenyl groups include ethenyl (C₂), 1-propenyl (C₃), 2-propenyl (C₃), 1-butenyl (C₄), 2-butenyl (C₄), butadienyl (C₄), and the like. Examples of C₂₋₆ alkenyl groups include the aforementioned C₂₋₄ alkenyl groups as well as pentenyl (C₅), pentadienyl (C₅), hexenyl (C₆), and the like. Additional examples of alkenyl include heptenyl (C₇), octenyl (C₈), octatrienyl (C₈), and the like.

“Alkynyl” refers to a radical of a straight-chain or branched hydrocarbon group having from 2 to 10 carbon atoms and one or more carbon-carbon triple bonds (e.g., 1, 2, 3, or 4 triple bonds) (“C₂₋₁₀ alkynyl”). In some embodiments, an alkynyl group has 2 to 9 carbon atoms (“C₂₋₉ alkynyl”). In some embodiments, an alkynyl group has 2 to 8 carbon atoms (“C₂₋₈ alkynyl”). In some embodiments, an alkynyl group has 2 to 7 carbon atoms (“C₂₋₇ alkynyl”). In some embodiments, an alkynyl group has 2 to 6 carbon atoms (“C₂₋₆ alkynyl”). In some embodiments, an alkynyl group has 2 to 5 carbon atoms (“C₂₋₅ alkynyl”). In some embodiments, an alkynyl group has 2 to 4 carbon atoms (“C₂₋₄ alkynyl”). In some embodiments, an alkynyl group has 2 to 3 carbon atoms (“C₂₋₃ alkynyl”). In some embodiments, an alkynyl group has 2 carbon atoms (“C₂ alkynyl”). The one or more carbon-carbon triple bonds can be internal (such as in 2-butynyl) or terminal (such as in 1-butynyl). Examples of C₂₋₄ alkynyl groups include, without limitation, ethynyl (C₂), 1-propynyl (C₃), 2-propynyl (C₃), 1-butynyl (C₄), 2-butynyl (C₄), and the like. Examples of C₂₋₆ alkenyl groups include the aforementioned C₂₋₄ alkynyl groups as well as pentynyl (Cs), hexynyl (C₆), and the like. Additional examples of alkynyl include heptynyl (C₇), octynyl (C₈), and the like.

“Carbocyclyl” or “carbocyclic” refers to a radical of a non-aromatic cyclic hydrocarbon group having from 3 to 14 ring carbon atoms (“C₃₋₁₄ carbocyclyl”) and zero heteroatoms in the non-aromatic ring system. In some embodiments, a carbocyclyl group has 3 to 10 ring carbon atoms (“C₃₋₁₀ carbocyclyl”). In some embodiments, a carbocyclyl group has 3 to 8 ring carbon atoms (“C₃₋₈ carbocyclyl”). In some embodiments, a carbocyclyl group has 3 to 7 ring carbon atoms (“C₃₋₇ carbocyclyl”). In some embodiments, a carbocyclyl group has 3 to 6 ring carbon atoms (“C₃₋₆ carbocyclyl”). In some embodiments, a carbocyclyl group has 4 to 6 ring carbon atoms (“C₄₋₆ carbocyclyl”). In some embodiments, a carbocyclyl group has 5 to 6 ring carbon atoms (“C₅₋₆ carbocyclyl”). In some embodiments, a carbocyclyl group has 5 to 10 ring carbon atoms (“C₅₋₁₀ carbocyclyl”). Exemplary C₃₋₆ carbocyclyl groups include, without limitation, cyclopropyl (C₃), cyclopropenyl (C₃), cyclobutyl (C₄), cyclobutenyl (C₄), cyclopentyl (C₅), cyclopentenyl (C₅), cyclohexyl (C₆), cyclohexenyl (C₆), cyclohexadienyl (C₆), and the like. Exemplary C₃₋₈ carbocyclyl groups include, without limitation, the aforementioned C₃₋₆ carbocyclyl groups as well as cycloheptyl (C₇), cycloheptenyl (C₇), cycloheptadienyl (C₇), cycloheptatrienyl (C₇), cyclooctyl (C₈), cyclooctenyl (C₈), bicyclo[2.2.1]heptanyl (C₇), bicyclo[2.2.2] octanyl (C₈), and the like. Exemplary C₃₋₁₀ carbocyclyl groups include, without limitation, the aforementioned C₃₋₈ carbocyclyl groups as well as cyclononyl (C₉), cyclononenyl (C₉), cyclodecyl (C₁₀), cyclodecenyl (C₁₀), octahydro-1H-indenyl (C₉), decahydronaphthalenyl (C₁₀), spiro[4.5]decanyl (C₁₀), and the like. As the foregoing examples illustrate, in certain embodiments, the carbocyclyl group is either monocyclic (“monocyclic carbocyclyl”) or polycyclic (e.g., containing a fused, bridged or spiro ring system such as a bicyclic system (“bicyclic carbocyclyl”) or tricyclic system (“tricyclic carbocyclyl”)) and can be saturated or can contain one or more carbon-carbon double or triple bonds. “Carbocyclyl” also includes ring systems wherein the carbocyclyl ring, as defined above, is fused with one or more aryl or heteroaryl groups wherein the point of attachment is on the carbocyclyl ring, and in such instances, the number of carbons continue to designate the number of carbons in the carbocyclic ring system.

In some embodiments, “carbocyclyl” is a monocyclic, saturated carbocyclyl group having from 3 to 14 ring carbon atoms (“C₃₋₁₄ cycloalkyl”). In some embodiments, “carbocyclyl” is a monocyclic, saturated carbocyclyl group having from 3 to 10 ring carbon atoms (“C₃₋₁₀ cycloalkyl”). In some embodiments, a cycloalkyl group has 3 to 8 ring carbon atoms (“C₃₋₈ cycloalkyl”). In some embodiments, a cycloalkyl group has 3 to 6 ring carbon atoms (“C₃₋₆ cycloalkyl”). In some embodiments, a cycloalkyl group has 4 to 6 ring carbon atoms (“C₄₋₆ cycloalkyl”). In some embodiments, a cycloalkyl group has 5 to 6 ring carbon atoms (“C₅₋₆ cycloalkyl”). In some embodiments, a cycloalkyl group has 5 to 10 ring carbon atoms (“C₅₋₁₀ cycloalkyl”). Examples of C₅₋₆ cycloalkyl groups include cyclopentyl (C₅) and cyclohexyl (C₈). Examples of C₃₋₆ cycloalkyl groups include the aforementioned C₅₋₆ cycloalkyl groups as well as cyclopropyl (C₃) and cyclobutyl (C₄). Examples of C₃₋₈ cycloalkyl groups include the aforementioned C₃₋₆ cycloalkyl groups as well as cycloheptyl (C₇) and cyclooctyl (C₈).

“Heterocyclyl” or “heterocyclic” refers to a group or radical of a 3- to 14-membered non-aromatic ring system having ring carbon atoms and 1 to 4 ring heteroatoms, wherein each heteroatom is independently selected from nitrogen, oxygen, and sulfur (“3-14 membered heterocyclyl”). In heterocyclyl groups that contain one or more nitrogen atoms, the point of attachment can be a carbon or nitrogen atom, as valency permits. A heterocyclyl group can either be monocyclic (“monocyclic heterocyclyl”) or polycyclic (e.g., a fused, bridged or spiro ring system such as a bicyclic system (“bicyclic heterocyclyl”) or tricyclic system (“tricyclic heterocyclyl”)), and can be saturated or can contain one or more carbon-carbon double or triple bonds. Heterocyclyl polycyclic ring systems can include one or more heteroatoms in one or both rings. “Heterocyclyl” also includes ring systems wherein the heterocyclyl ring, as defined above, is fused with one or more carbocyclyl groups wherein the point of attachment is either on the carbocyclyl or heterocyclyl ring, or ring systems wherein the heterocyclyl ring, as defined above, is fused with one or more aryl or heteroaryl groups, wherein the point of attachment is on the heterocyclyl ring, and in such instances, the number of ring members continue to designate the number of ring members in the heterocyclyl ring system.

In some embodiments, a heterocyclyl group is a 5-10 membered non-aromatic ring system having ring carbon atoms and 1-4 ring heteroatoms, wherein each heteroatom is independently selected from nitrogen, oxygen, and sulfur (“5-10 membered heterocyclyl”). In some embodiments, a heterocyclyl group is a 5-8 membered non-aromatic ring system having ring carbon atoms and 1-4 ring heteroatoms, wherein each heteroatom is independently selected from nitrogen, oxygen, and sulfur (“5-8 membered heterocyclyl”). In some embodiments, a heterocyclyl group is a 5-6 membered non-aromatic ring system having ring carbon atoms and 1-4 ring heteroatoms, wherein each heteroatom is independently selected from nitrogen, oxygen, and sulfur (“5-6 membered heterocyclyl”). In some embodiments, the 5-6 membered heterocyclyl has 1-3 ring heteroatoms selected from nitrogen, oxygen, and sulfur. In some embodiments, the 5-6 membered heterocyclyl has 1-2 ring heteroatoms selected from nitrogen, oxygen, and sulfur. In some embodiments, the 5-6 membered heterocyclyl has 1 ring heteroatom selected from nitrogen, oxygen, and sulfur.

Exemplary 3-membered heterocyclyl groups containing 1 heteroatom include, without limitation, azirdinyl, oxiranyl, and thiiranyl. Exemplary 4-membered heterocyclyl groups containing 1 heteroatom include, without limitation, azetidinyl, oxetanyl and thietanyl. Exemplary 5-membered heterocyclyl groups containing 1 heteroatom include, without limitation, tetrahydrofuranyl, dihydrofuranyl, tetrahydrothiophenyl, dihydrothiophenyl, pyrrolidinyl, dihydropyrrolyl, and pyrrolyl-2,5-dione. Exemplary 5-membered heterocyclyl groups containing 2 heteroatoms include, without limitation, dioxolanyl, oxathiolanyl and dithiolanyl. Exemplary 5-membered heterocyclyl groups containing 3 heteroatoms include, without limitation, triazolinyl, oxadiazolinyl, and thiadiazolinyl. Exemplary 6-membered heterocyclyl groups containing 1 heteroatom include, without limitation, piperidinyl, tetrahydropyranyl, dihydropyridinyl, and thianyl. Exemplary 6-membered heterocyclyl groups containing 2 heteroatoms include, without limitation, piperazinyl, morpholinyl, dithianyl, and dioxanyl. Exemplary 6-membered heterocyclyl groups containing 3 heteroatoms include, without limitation, triazinanyl. Exemplary 7-membered heterocyclyl groups containing 1 heteroatom include, without limitation, azepanyl, oxepanyl and thiepanyl. Exemplary 8-membered heterocyclyl groups containing 1 heteroatom include, without limitation, azocanyl, oxecanyl and thiocanyl. Exemplary bicyclic heterocyclyl groups include, without limitation, indolinyl, isoindolinyl, dihydrobenzofuranyl, dihydrobenzothienyl, tetrahydrobenzothienyl, tetrahydrobenzofuranyl, tetrahydroindolyl, tetrahydroquinolinyl, tetrahydroisoquinolinyl, decahydroquinolinyl, decahydroisoquinolinyl, octahydrochromenyl, octahydroisochromenyl, decahydronaphthyridinyl, decahydro-1,8-naphthyridinyl, octahydropyrrolo[3,2-b]pyrrole, indolinyl, phthalimidyl, naphthalimidyl, chromanyl, chromenyl, 1H-benzo[e][1,4]diazepinyl, 1,4,5,7-tetrahydropyrano[3,4-b]pyrrolyl, 5,6-dihydro-4H-furo[3,2-b]pyrrolyl, 6,7-dihydro-5H-furo[3,2-b]pyranyl, 5,7-dihydro-4H-thieno[2,3-c]pyranyl, 2,3-dihydro-1H-pyrrolo[2,3-b]pyridinyl, 2,3-dihydrofuro[2,3-b]pyridinyl, 4,5,6,7-tetrahydro-1H-pyrrolo-[2,3-b]pyridinyl, 4,5,6,7-tetrahydrofuro[3,2-c]pyridinyl, 4,5,6, 7-tetrahydrothieno[3,2-b]pyridinyl, 1,2,3,4-tetrahydro-1,6-naphthyridinyl, and the like.

“Aryl” refers to a radical of a monocyclic or polycyclic (e.g., bicyclic or tricyclic) 4n+2 aromatic ring system (e.g., having 6, 10, or 14 pi electrons shared in a cyclic array) having 6-14 ring carbon atoms and zero heteroatoms provided in the aromatic ring system (“C₆₋₁₄ aryl”). In some embodiments, an aryl group has 6 ring carbon atoms (“C₆ aryl”; e.g., phenyl). In some embodiments, an aryl group has 10 ring carbon atoms (“C₁₀ aryl”; e.g., naphthyl such as 1-naphthyl (α-naphthyl) and 2-naphthyl (β-naphthyl)). In some embodiments, an aryl group has 14 ring carbon atoms (“C₁₄ aryl”; e.g., anthracyl). “Aryl” also includes ring systems wherein the aryl ring, as defined above, is fused with one or more carbocyclyl or heterocyclyl groups wherein the radical or point of attachment is on the aryl ring, and in such instances, the number of carbon atoms continue to designate the number of carbon atoms in the aryl ring system.

“Heteroaryl” refers to a radical of a 5-14 membered monocyclic or polycyclic (e.g., bicyclic, tricyclic) 4n+2 aromatic ring system (e.g., having 6, 10, or 14 pi electrons shared in a cyclic array) having ring carbon atoms and 1-4 ring heteroatoms provided in the aromatic ring system, wherein each heteroatom is independently selected from nitrogen, oxygen, and sulfur (“5-14 membered heteroaryl”). In heteroaryl groups that contain one or more nitrogen atoms, the point of attachment can be a carbon or nitrogen atom, as valency permits. Heteroaryl polycyclic ring systems can include one or more heteroatoms in one or both rings. “Heteroaryl” includes ring systems wherein the heteroaryl ring, as defined above, is fused with one or more carbocyclyl or heterocyclyl groups wherein the point of attachment is on the heteroaryl ring, and in such instances, the number of ring members continue to designate the number of ring members in the heteroaryl ring system. “Heteroaryl” also includes ring systems wherein the heteroaryl ring, as defined above, is fused with one or more aryl groups wherein the point of attachment is either on the aryl or heteroaryl ring, and in such instances, the number of ring members designates the number of ring members in the fused polycyclic (aryl/heteroaryl) ring system. Polycyclic heteroaryl groups wherein one ring does not contain a heteroatom (e.g., indolyl, quinolinyl, carbazolyl, and the like) the point of attachment can be on either ring, i.e., either the ring bearing a heteroatom (e.g., 2-indolyl) or the ring that does not contain a heteroatom (e.g., 5-indolyl).

In some embodiments, a heteroaryl group is a 5-10 membered aromatic ring system having ring carbon atoms and 1-4 ring heteroatoms provided in the aromatic ring system, wherein each heteroatom is independently selected from nitrogen, oxygen, and sulfur (“5-10 membered heteroaryl”). In some embodiments, a heteroaryl group is a 5-8 membered aromatic ring system having ring carbon atoms and 1-4 ring heteroatoms provided in the aromatic ring system, wherein each heteroatom is independently selected from nitrogen, oxygen, and sulfur (“5-8 membered heteroaryl”). In some embodiments, a heteroaryl group is a 5-6 membered aromatic ring system having ring carbon atoms and 1-4 ring heteroatoms provided in the aromatic ring system, wherein each heteroatom is independently selected from nitrogen, oxygen, and sulfur (“5-6 membered heteroaryl”). In some embodiments, the 5-6 membered heteroaryl has 1-3 ring heteroatoms selected from nitrogen, oxygen, and sulfur. In some embodiments, the 5-6 membered heteroaryl has 1-2 ring heteroatoms selected from nitrogen, oxygen, and sulfur. In some embodiments, the 5-6 membered heteroaryl has 1 ring heteroatom selected from nitrogen, oxygen, and sulfur.

Exemplary 5-membered heteroaryl groups containing 1 heteroatom include, without limitation, pyrrolyl, furanyl and thiophenyl. Exemplary 5-membered heteroaryl groups containing 2 heteroatoms include, without limitation, imidazolyl, pyrazolyl, oxazolyl, isoxazolyl, thiazolyl, and isothiazolyl. Exemplary 5-membered heteroaryl groups containing 3 heteroatoms include, without limitation, triazolyl, oxadiazolyl, and thiadiazolyl. Exemplary 5-membered heteroaryl groups containing 4 heteroatoms include, without limitation, tetrazolyl. Exemplary 6-membered heteroaryl groups containing 1 heteroatom include, without limitation, pyridinyl. Exemplary 6-membered heteroaryl groups containing 2 heteroatoms include, without limitation, pyridazinyl, pyrimidinyl, and pyrazinyl. Exemplary 6-membered heteroaryl groups containing 3 or 4 heteroatoms include, without limitation, triazinyl and tetrazinyl, respectively. Exemplary 7-membered heteroaryl groups containing 1 heteroatom include, without limitation, azepinyl, oxepinyl, and thiepinyl. Exemplary 5,6-bicyclic heteroaryl groups include, without limitation, indolyl, isoindolyl, indazolyl, benzotriazolyl, benzothiophenyl, isobenzothiophenyl, benzofuranyl, benzoisofuranyl, benzimidazolyl, benzoxazolyl, benzisoxazolyl, benzoxadiazolyl, benzthiazolyl, benzisothiazolyl, benzthiadiazolyl, indolizinyl, and purinyl. Exemplary 6,6-bicyclic heteroaryl groups include, without limitation, naphthyridinyl, pteridinyl, quinolinyl, isoquinolinyl, cinnolinyl, quinoxalinyl, phthalazinyl, and quinazolinyl. Exemplary tricyclic heteroaryl groups include, without limitation, phenanthridinyl, dibenzofuranyl, carbazolyl, acridinyl, phenothiazinyl, phenoxazinyl and phenazinyl.

“Saturated” refers to a ring moiety that does not contain a double or triple bond, i.e., the ring contains all single bonds.

Alkyl, cycloalkyl, heterocyclyl, aryl, and heteroaryl groups may be optionally substituted. Optionally substituted refers to a group which may be substituted or unsubstituted. In general, the term “substituted” means that at least one hydrogen present on a group is replaced with a non-hydrogen substituent, and which upon substitution results in a stable compound, e.g., a compound which does not spontaneously undergo transformation such as by rearrangement, cyclization, elimination, or other reaction. Heteroatoms such as nitrogen, oxygen, and sulfur may have hydrogen substituents and/or non-hydrogen substituents which satisfy the valencies of the heteroatoms and results in the formation of a stable compound.

Exemplary non-hydrogen substituents may be selected from the group consisting of halogen, —CN, —NO₂, —N₃, —SO₂H, —SO₃H, —OH, —OR^{aa}, —N(R^{bb})₂, —N(OR^{cc})R^{bb}, —SH, —SR^{aa}, —C(═O)R^{aa}, —CO₂H, —CHO, —CO₂R^{aa}, —OC(═O)R^{aa}, —OCO₂R^{aa}, —C(═O)N(R^{bb})₂, —OC(═O)N(R^{bb})₂, —NR^{bb}C(═O)R^{aa}, —NR^{bb}CO₂R^{aa}, —NR^{bb}C(═O)N(R^{bb})₂, —C(═NR^{bb})R^{aa}, —C(═NR^{bb})OR^{aa}, —OC(═NR^{bb})R^{aa}, —OC(═NR^{bb})OR^{aa}, —C(═NR^{bb})N(R^{bb})₂, —OC(═NR^{bb})N(R^{bb})₂, —NR^{bb}C(═NR^{bb})N(R^{bb})₂, —C(—O)NR^{bb}SO₂R^{aa}, —NR^{bb}SO₂R^{aa}, —SO₂N(R^{bb})₂, —SO₂R^{aa}, —S(═O)R^{aa}, —OS(═O)R^{aa}, —B(OR^{cc})₂, C₁₋₁₀ alkyl, C₂₋₁₀ alkenyl, C₂₋₁₀ alkynyl, C₃₋₁₄ carbocyclyl, 3- to 14-membered heterocyclyl, C₆₋₁₄ aryl, and 5- to 14-membered heteroaryl, wherein each alkyl, alkenyl, alkynyl, carbocyclyl, heterocyclyl, aryl, and heteroaryl is independently substituted with 0, 1, 2, 3, 4, or 5 R^{dd} groups, or two geminal hydrogens on a carbon atom are replaced with the group ═O;
each instance of R^{aa} is, independently, selected from the group consisting of C₁₋₁₀ alkyl, C₁₋₁₀ perhaloalkyl, C₂₋₁₀ alkenyl, C₂₋₁₀ alkynyl, C₃₋₁₄ carbocyclyl, 3- to 14-membered heterocyclyl, C₆₋₁₄ aryl, and 5- to 14-membered heteroaryl, or two R^{aa} groups are joined to form a 3- to 14-membered heterocyclyl or 5- to 14-membered heteroaryl ring, wherein each alkyl, alkenyl, alkynyl, carbocyclyl, heterocyclyl, aryl, and heteroaryl is independently substituted with 0, 1, 2, 3, 4, or 5 R^{dd} groups;
each instance of R^{bb} is, independently, selected from the group consisting of hydrogen, —OH, —OR^{aa}, —N(R^{cc})₂, —CN, —C(═O)R^{aa}, —C(═O)N(R^{cc})₂, —CO₂R^{aa}, —SO₂R^{aa}, —SO₂N(R^{cc})₂, —SOR^{aa}, C_{1 10} alkyl, C₁₋₁₀ perhaloalkyl, C₂₋₁₀ alkenyl, C₂₋₁₀ alkynyl, C₃₋₁₄ carbocyclyl, 3- to 14-membered heterocyclyl, C₆₋₁₄ aryl, and 5- to 14-membered heteroaryl, or two R^{bb} groups are joined to form a 3- to 14-membered heterocyclyl or 5- to 14-membered heteroaryl ring, wherein each alkyl, alkenyl, alkynyl, carbocyclyl, heterocyclyl, aryl, and heteroaryl is independently substituted with 0, 1, 2, 3, 4, or 5 R^{dd} groups;
each instance of R^{cc} is, independently, selected from the group consisting of hydrogen, C₁₋₁₀ alkyl, C₁₋₁₀ perhaloalkyl, C₂₋₁₀ alkenyl, C₂₋₁₀ alkynyl, C₃₋₁₄ carbocyclyl, 3- to 14-membered heterocyclyl, C₆₋₁₄ aryl, and 5- to 14-membered heteroaryl, or two R^{cc} groups are joined to form a 3- to 14-membered heterocyclyl or 5- to 14-membered heteroaryl ring, wherein each alkyl, alkenyl, alkynyl, carbocyclyl, heterocyclyl, aryl, and heteroaryl is independently substituted with 0, 1, 2, 3, 4, or 5 R^{dd} groups; and
each instance of R^{dd} is, independently, selected from the group consisting of halogen, —CN, —NO₂, —N₃, —SO₂H, —SO₃H, —OH, —OC₁₋₆ alkyl, —ON(C₁₋₁₀alkyl)₂, —N (C₁₋₆ alkyl)₂, —N(OC₁₋₆ alkyl)(C₁₋₆ alkyl), —N(OH)(C₁₋₆ alkyl), —NH (OH), —SH, —SC₁₋₆ alkyl, —C(═O)(C₁₋₆ alkyl), —CO₂H, —CO₂(C₁₋₆ alkyl), —OC(═O)(C₁₋₆ alkyl), —OCO₂(C₁₋₆ alkyl), —C(═O)NH₂, —C(═O)N(C₁₋₆ alkyl)₂, —OC(═O)NH(C₁₋₆ alkyl), —NHC(═O)(C₁₋₆ alkyl), —N (C₁₋₆alkyl)C(═O)(C₁₋₆ alkyl), —NHCO₂(C₁₋₆ alkyl), —NHC(═O)N(C₁₋₆ alkyl)₂, —NHC(═O)NH(C₁₋₆ alkyl), —NHC(═O)NH₂, —C(═NH)O(C₁₋₆ alkyl), —OC(═NH)(C₁₋₆ alkyl), —OC(═NH)OC₁₋₆ alkyl, —C (═NH)N(C₁₋₆ alkyl)₂, —C(═NH)NH(C₁₋₆ alkyl), —C(═NH)NH₂, —OC(═NH)N(C₁₋₆ alkyl)₂, —OC(NH)NH(C₁₋₆ alkyl), —OC(NH)NH₂, —NHC(NH)N(C₁₋₆ alkyl)₂, —NHC(═NH)NH₂, —NHSO₂(C₁₋₆ alkyl), —SO₂N(C₁₋₆ alkyl)₂, —SO₂NH(C₁₋₆ alkyl), —SO₂NH₂, —SO₂C₁₋₆ alkyl, —B(OH)₂, —B(OC₁₋₆ alkyl)₂, C₁₋₆ alkyl, C₁₋₆ perhaloalkyl, C₂₋₆ alkenyl, C₂₋₆ alkynyl, C₃₋₁₀ carbocyclyl, C_{6 10} aryl, 3- to 10-membered heterocyclyl, and 5- to 10-membered heteroaryl; or two geminal R^{dd} substituents on a carbon atom may be joined to form ═O.

“Halo” or “halogen” refers to fluorine (fluoro, —F), chlorine (chloro, —Cl), bromine (bromo, —Br), or iodine (iodo, —I).

It should be noted that in hetero-atom containing ring systems described herein, there are no hydroxyl groups on carbon atoms adjacent to a N, O or S, as well as there are no N or S groups on carbon adjacent to another heteroatom. Thus, for example, in the ring:

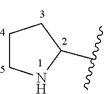 [SMILES: *C1CCCN1]

there is no —OH attached directly to carbons marked 2 and 5.

It should also be noted that tautomeric forms such as, for example, the moieties:

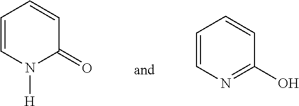 [SMILES: O=c1cccc[nH]1 ; Oc1ccccn1]

are considered equivalent unless otherwise specified.

### EMBODIMENTS

Examples of embodiments of the present application include the following:

### Embodiment 1

A process for preparing a compound of Formula (I)

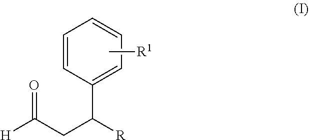

wherein:
R is C₁-C₁₀ alkyl that is optionally substituted with one substituent selected from the group consisting of C₃-C₇ cycloalkyl, C₆-C₁₀ aryl, halo, —O—C(═O)-(C₁-C₆ alkyl), —O-(C₆-C₁₀ aryl), and —O-(C₁-C₆ alkyl) wherein the C₁-C₆ alkyl of the —O-(C₁-C₆ alkyl) is optionally substituted with C₆-C₁₀ aryl; C₃-C₇cycloalkyl; and C₆-C₁₀ aryl;
R1 is hydrogen, halo, —C (═O)-(C₁-C₆ alkyl), halo (C₁-C₆ alkyl), C₁-C₆ alkyl, —NO₂, —C(═O)—OH, —CN, —C(═O)—O-(C₁-C₆ alkyl), and —O-(C₁-C₆ alkyl), or —O-halo (C₁-C₆ alkyl); comprising reacting a compound of Formula (II)

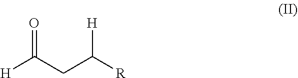

wherein R in Formula (II) is as defined above for Formula (I);
with a compound of Formula (III)

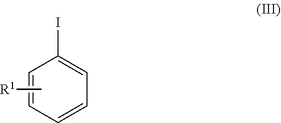

wherein R¹ in Formula (III) is as defined above for Formula (I);
in the presence of a palladium salt, an amino acid based transient directing group compound (TDG), a 2-pyridone ligand (L), a salt composition comprising at least one silver salt, and an acid composition comprising acetic acid or an acetic acid derivative.

Both alpha and beta amino acids based transient directing group compounds (TDG) are contemplated.

### Embodiment 2

The process according to Embodiment 1, wherein in R:
the optionally substituted C₁-C₁₀ alkyl is selected from the group consisting of —(CH₂)-n-C₆H₁₃, —CH₂CH₃, —CH₂CH₂CH₃, —CH₂-cyclohexyl, —CH₂CH₂-phenyl, —(CH₂)₄-CH₂F, —(CH₂)₂CH₂-naphthyl, —(CH₂)₂CH₂-O—C(═O)—CH₃, —(CH₂)₃CH₂-O-phenyl, —(CH₂)₄CH₂—O-n-C₃H₇, —(CH₂)₄CH₂—O—CH(CH₃)₂, —(CH₂)₄CH₂-O-benzyl, and —(CH₂)₄CH₂-O-naphthyl;
the C₃-C₇cycloalkyl is cyclohexyl; and
the C₆-C₁₀ aryl is phenyl.

### Embodiment 3

The process according to Embodiment 1 or 2, wherein in R¹:
the —C(═O)-(C₁-C₆ alkyl) is —C(═O)—CH₃;
the halo(C₁-C₆ alkyl) is CF₃;
the —C(═O)—O—(C₁-C₆ alkyl) is —C(═O)—OCH₃;
the —O-(C₁-C₆ alkyl) is —OCH₃; and
the —O-halo(C₁-C₆ alkyl) is —OCF₃.

### Embodiment 4

The process according to any one of Embodiments 1-3, wherein the compound of Formula (I) is selected from the group consisting of:

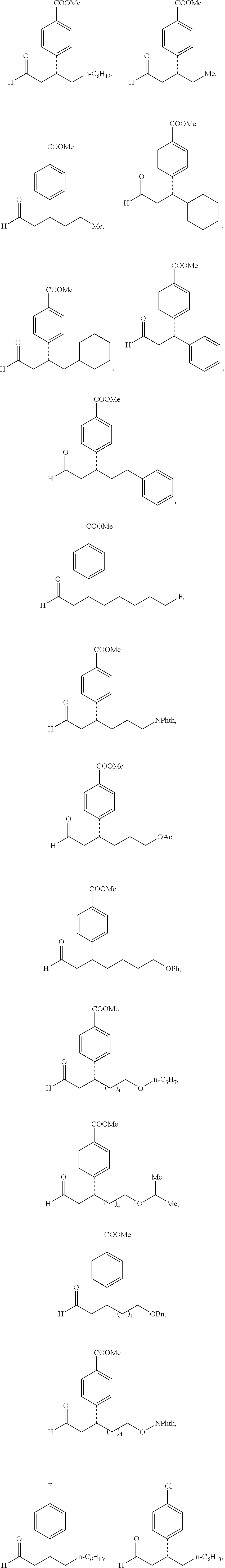

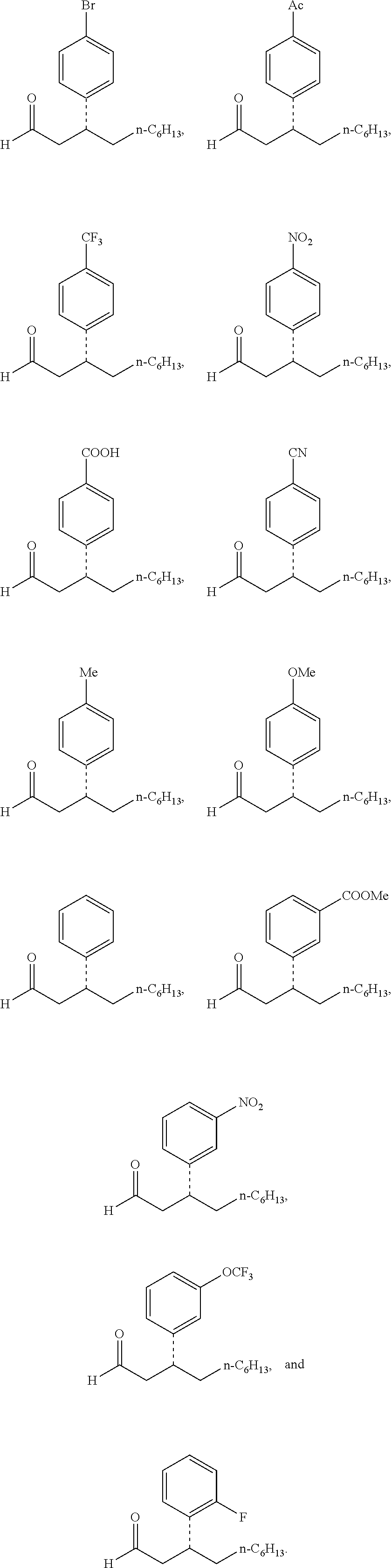

### Embodiment 5

The process according to any one of Embodiments 1-4, wherein the palladium salt is Pd(OAc)₂, Pd(TFA)₂, or PdCl₂.

### Embodiment 6

The process according to any one of Embodiments 1-5, wherein the TDG is selected from the group consisting of

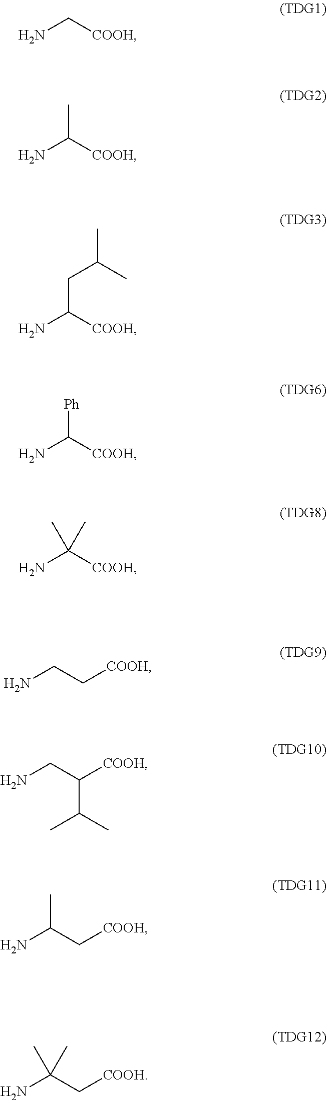

### Embodiment 7

The process according to Embodiment 6, wherein the TDG is TDG12.

### Embodiment 8

The process according to any one of Embodiments 1-7, wherein L is selected from the group consisting of

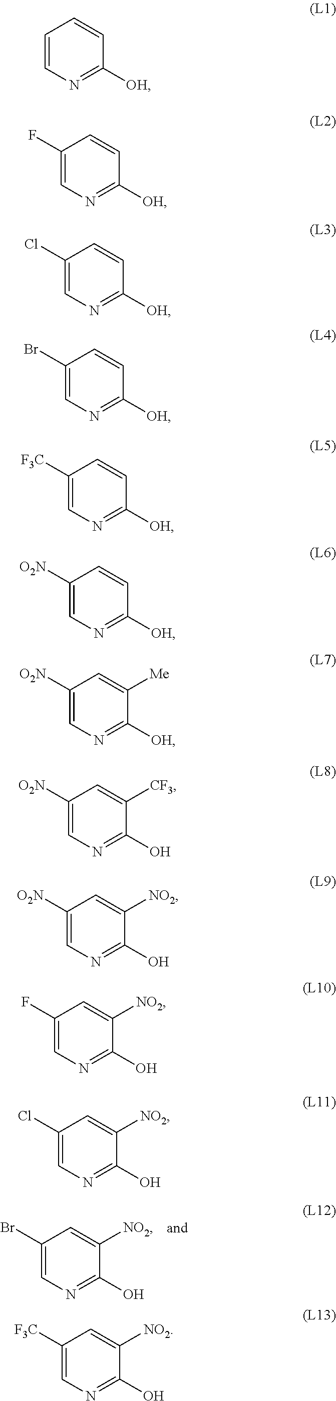

### Embodiment 9

The process according to Embodiment 8, wherein L is L8.

### Embodiment 10

The process according to any one of Embodiments 1-9, wherein the at least one silver salt of the salt composition is selected from the group consisting of silver trifluoroacetate (AgTFA), silver carbonate (Ag₂CO₃), silver acetate (AgOAc), and silver nitrate (AgNO₃).

### Embodiment 11

The process according to Embodiment 10, wherein the salt composition further comprises in addition to the silver salt, a metal salt distinct from the silver salt.

### Embodiment 12

The process according to Embodiment 11, wherein the metal salt distinct from the silver salt is selected from the group consisting of sodium carbonate (Na₂CO₃), lithium carbonate (Li₂CO₃), and cesium carbonate (Cs₂CO₃).

### Embodiment 13

The process according to any one of Embodiments 10-12, wherein the salt composition is selected from the group consisting of: a mixture of AgTFA and Ag₂CO₃, a mixture of AgTFA and Ag₂O, a mixture of AgTFA and Li₂CO₃, and a mixture of AgTFA and AgNO₃.

### Embodiment 14

The process according to any one of Embodiments 1-13, wherein the acetic acid derivative of the acid composition is selected from the group consisting of chloroacetic acid (ClCH₂COOH), trichloroacetic acid (Cl₃CCOOH), difluoroacetic acid (F₂CHCOOH), and trifluoroacetic acid (F₃CCOOH).

### Embodiment 15

A process for preparing a compound of Formula (IV)

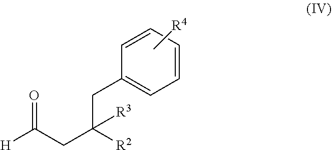

wherein:
R² is H, or C₁-C₆ alkyl;
R³ is C₃-C₇ cycloalkyl; or C₁-C₆ alkyl which is optionally substituted with a substituent selected from the group consisting of —O—C(—O)-C₁-C₆ alkyl and C₆-C₁₀ aryl; and
R⁴ is selected from the group consisting of —C(═O)-(C₁-C₆ alkyl); —C(═O)—O—(C₁-C₆ alkyl); —C(═O)—O-(C₃-C₁₀ bicyclic carbocyclyl) wherein said C₃-C₁₀ bicyclic carbocyclyl is optionally substituted with C₁-C₆ alkyl; halo; halo (C₁-C₆ alkyl); —NO₂; —CN;

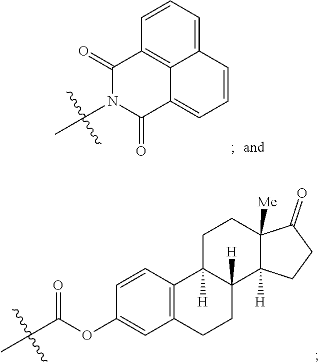

comprising reacting a compound of Formula (V)

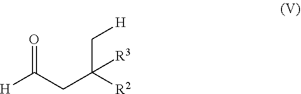

wherein R² and R³ in Formula (V) are as defined above for Formula (IV);
with a compound of Formula (VI)

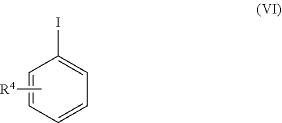

wherein R⁴ in Formula (VI) is as defined above for Formula (IV);
in the presence of a palladium salt, an amino acid based transient directing group compound (TDG), a 2-pyridone ligand (L), a salt composition comprising at least one silver salt, and an acid composition comprising acetic acid or an acetic acid derivative.

Both alpha and beta amino acids based transient directing group compounds (TDG) are contemplated.

### Embodiment 16

The process according to Embodiment 15, wherein:
in R², the C₁-C₆ alkyl is methyl;
in R³, the C₃-C₇ cycloalkyl is cyclopentyl or cyclohexyl; the optionally substituted C₁-C₆ alkyl is —CH₃, —(CH₂)₄CH₃, —CH₂-t-Butyl, —(CH₂)₃CH(CH₃)₂, —(CH₂)₂—O—C(═O)—CH₃, —(CH₂)₂-phenyl, or —(CH₂)₃CH₃; and
in R⁴, the —C(═O)-(C₁-C₆ alkyl) is —C(═O)—CH₃; the —C(═O)—O-(C₁-C₆ alkyl) is —C(═O)—O—CH₃; the —C(═O)—O-(C₃-C₁₀ bicyclic carbocyclyl) with the optionally substituted bicyclic carbocyclyl is

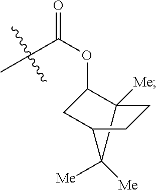 [SMILES: *C(=O)OC1CC2CCC1(C)C2(C)C]

and the halo (C₁-C₆ alkyl) is CF₃.

### Embodiment 17

The process according to Embodiment 15 or 16, wherein the compound of Formula (IV) is selected from the group consisting of:

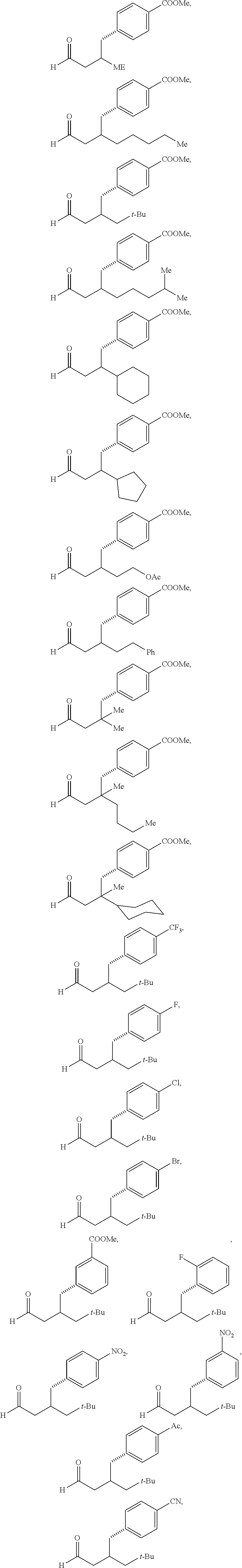

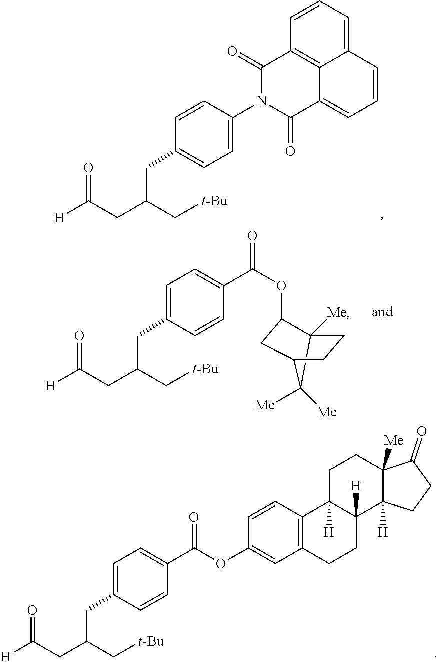

### Embodiment 18

The process according to any one of Embodiments 15-17, wherein the palladium salt is Pd(OAc₂, Pd(TFA)₂, or PdCl₂.

### Embodiment 19

The process according to any one of Embodiments 15-18, wherein the TDG is selected from the group consisting of

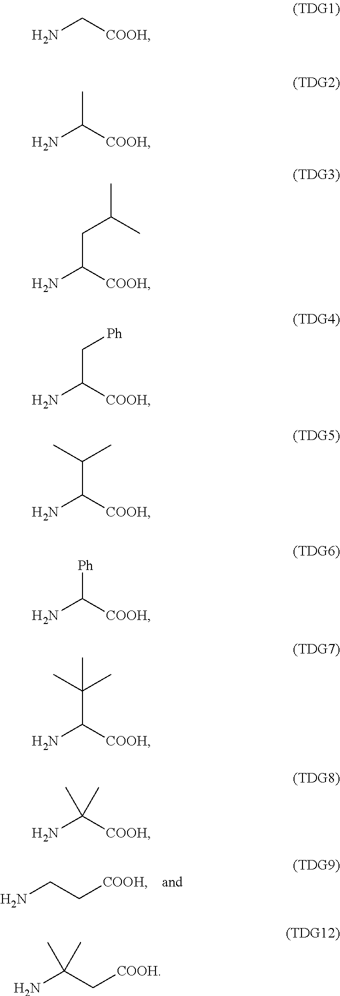

### Embodiment 20

The process according to Embodiment 19, wherein the TDG is TDG7.

### Embodiment 21

The process according to any one of Embodiments 15-20, wherein L is selected from the group consisting of

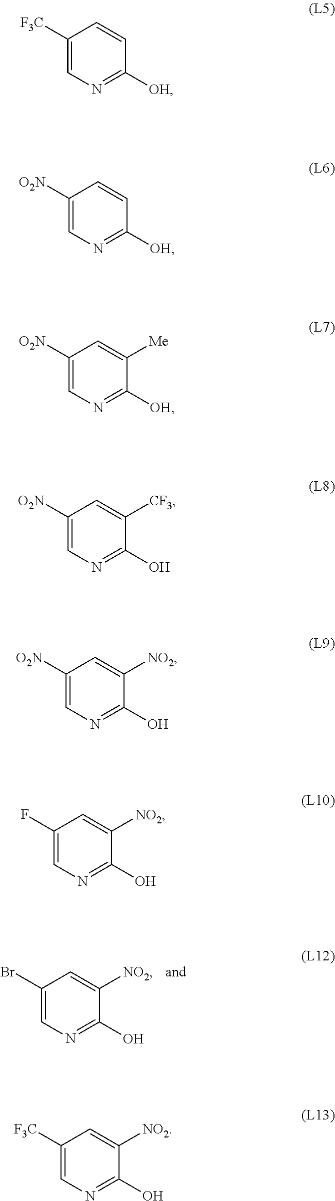

### Embodiment 22

The process according to Embodiment 21, wherein L is L8.

### Embodiment 23

The process according to any one of claims 15-22, wherein the at least one silver salt of the salt composition is selected from the group consisting of silver trifluoroacetate (AgTFA), silver carbonate (Ag₂CO₃), silver acetate (AgOAc), and silver nitrate (AgNO₃).

### Embodiment 24

The process according to Embodiment 15, wherein the salt composition further comprises in addition to the silver salt, a metal salt distinct from the silver salt.

### Embodiment 25

The process according to Embodiment 24, wherein the metal salt distinct from the silver salt is selected from the group consisting of sodium carbonate (Na₂CO₃), lithium carbonate (Li₂CO₃), and cesium carbonate (Cs₂CO₃).

### Embodiment 26

The process according to any one of Embodiments 23-25, wherein the salt composition is selected from the group consisting of: a mixture of AgTFA and Ag₂CO₃, a mixture of AgTFA and Ag₂O, a mixture of AgTFA and Li₂CO₃, and a mixture of AgTFA and AgNO₃.

### Embodiment 27

The process according to any one of Embodiments 15-26, wherein the acetic acid derivative of the acid composition is selected from the group consisting of chloroacetic acid (ClCH₂COOH), trichloroacetic acid (Cl₃CCOOH), difluoroacetic acid (F₂CHCOOH), and trifluoroacetic acid (F₃CCOOH).

### Embodiment 28

A process for preparing a compound of Formula (VII)

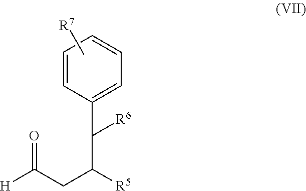

wherein:
R⁵ and R⁶ together with the carbon atoms to which they are shown attached form a C₄-C₇ cycloalkyl; and
R⁷ is selected from the group consisting of —C (═O)-(C₁-C₆ alkyl); —C(═O)—O-(C₁-C₆ alkyl); —C(═O)—O-(C₃-C₁₀ bicyclic carbocyclyl) wherein said C₃-C₁₀ bicyclic carbocyclyl is optionally substituted with C₁-C₆ alkyl; halo; halo (C₁-C₆ alkyl); —NO₂; —CN;

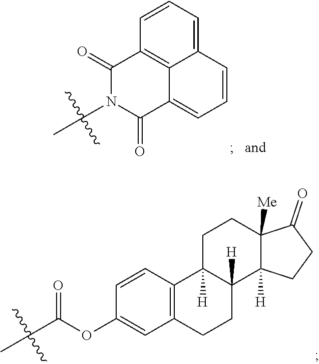

comprising reacting a compound of Formula (VII)

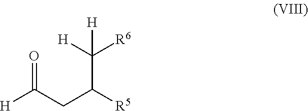

wherein R5 and R6 are as defined above for Formula (VII);
with a compound of Formula (IX)

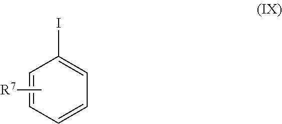

wherein R⁷ is as defined above for Formula (VII);
in the presence of a palladium salt, an amino acid based transient directing group compound (TDG), a 2-pyridone ligand (L), a salt composition comprising at least one silver salt, and an acid composition comprising acetic acid or an acetic acid derivative.

Both alpha and beta amino acids based transient directing group compounds (TDG) are contemplated.

### Embodiment 29

The process according to Embodiment 28, wherein:
R⁵ and R⁶ together with the carbon atoms to which they are shown attached form a cyclohexyl group; and
in R⁷, the —C(═O)-(C₁-C₆ alkyl) is —C(═O)—CH₃; the —C(═O)—O-(C₁-C₆ alkyl) is —C(═O)—O—CH₃; the —C(—O)—O-(C₃-C₁₀ bicyclic carbocyclyl) with the optionally substituted bicyclic carbocyclyl is

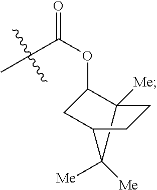 [SMILES: *C(=O)OC1CC2CCC1(C)C2(C)C]

and the halo (C₁-C₆ alkyl) is CF₃.

### Embodiment 30

The process according to Embodiment 29, wherein R⁷ is —C(═O)—O—CH₃.

### Embodiment 31

The process according to any one of Embodiments 28-30, wherein the compound of Formula (VII) is

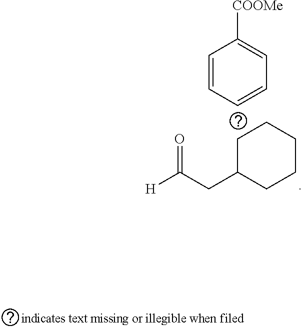

### Embodiment 32

The process according to any one of Embodiments 28-31, wherein the palladium salt is Pd(OAc)₂, Pd(TFA)₂, or PdCl₂.

### Embodiment 33

The process according to any one of Embodiments 28-32, wherein the TDG is selected from the group consisting of

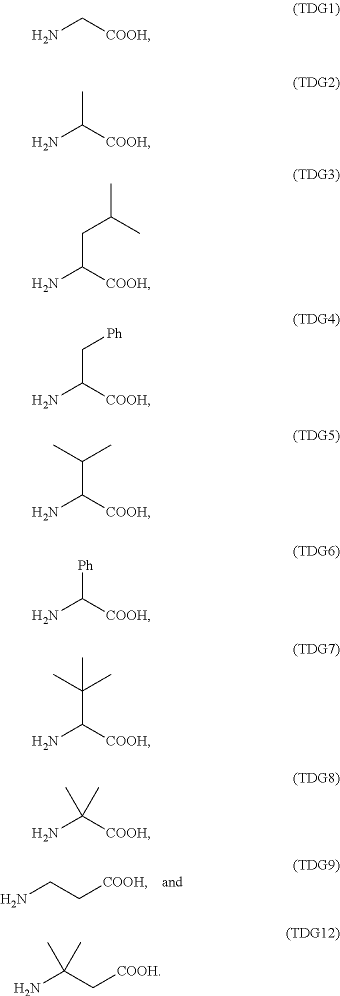

### Embodiment 34

The process according to Embodiment 33, wherein the TDG is TDG7.

### Embodiment 35

The process according to any one of Embodiments 28-34, wherein L is selected from the group consisting of

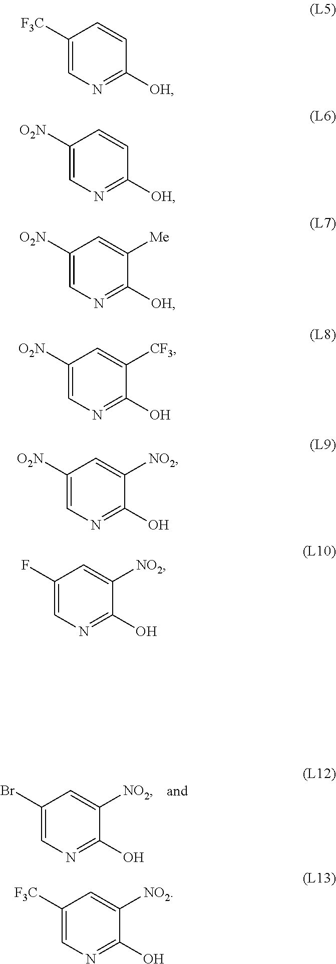

### Embodiment 36

The process according to Embodiment 35, wherein L is L8.

### Embodiment 37

The process according to any one of Embodiments 28-36, wherein the at least one silver salt of the salt composition is selected from the group consisting of silver trifluoroacetate (AgTFA), silver carbonate (Ag₂CO₃), silver acetate (AgOAc), and silver nitrate (AgNO₃).

### Embodiment 38

The process according to Embodiment 37, wherein the salt composition further comprises in addition to the silver salt, a metal salt distinct from the silver salt.

### Embodiment 39

The process according to Embodiment 38, wherein the metal salt distinct from the silver salt is selected from the group consisting of sodium carbonate (Na₂CO₃), lithium carbonate (Li₂CO₃), and cesium carbonate (Cs₂CO₃).

### Embodiment 40

The process according to any one of Embodiments 37-39, wherein the salt composition is selected from the group consisting of: a mixture of AgTFA and Ag₂CO₃, a mixture of AgTFA and Ag₂O, a mixture of AgTFA and Li₂CO₃, and a mixture of AgTFA and AgNO₃.

### Embodiment 41

The process according to any one of Embodiments 28-40, wherein the acetic acid derivative of the acid composition is selected from the group consisting of chloroacetic acid (ClCH₂COOH), trichloroacetic acid (Cl₃CCOOH), difluoroacetic acid (F₂CHCOOH), and trifluoroacetic acid (F₃CCOOH).

### EXAMPLES

### General Examples for the Processes of the Invention

Herein, we report a combination of ligand and TDG that enabled site-selective Pd^{II}-catalyzed C(sp³)-H arylation of a broad range of primary aldehydes (Scheme 1c). With 3-amino-3-methylbutyric acid (TDG12) as transient directing group, β-methylene C—H arylation could be achieved with up to 83% yield. By simply employing a different TDG7, tert-Leucine, the regioselectivity could be switched to relative remote y-position. Mechanistic studies combined with density functional theory (DFT) calculations suggested that matching the TDG bite angle with the size of palladacycle could minimize the strain in the C—H activation transition state (TS), thereby controlling the site-selectivity. Considering the recent extensive examples of remote C(sp²)—H activation reactions developed using distance and geometry as the core parameters,⁸ this finding represents a promising step towards systematical development of remote site-selective C(sp³)—H activation.

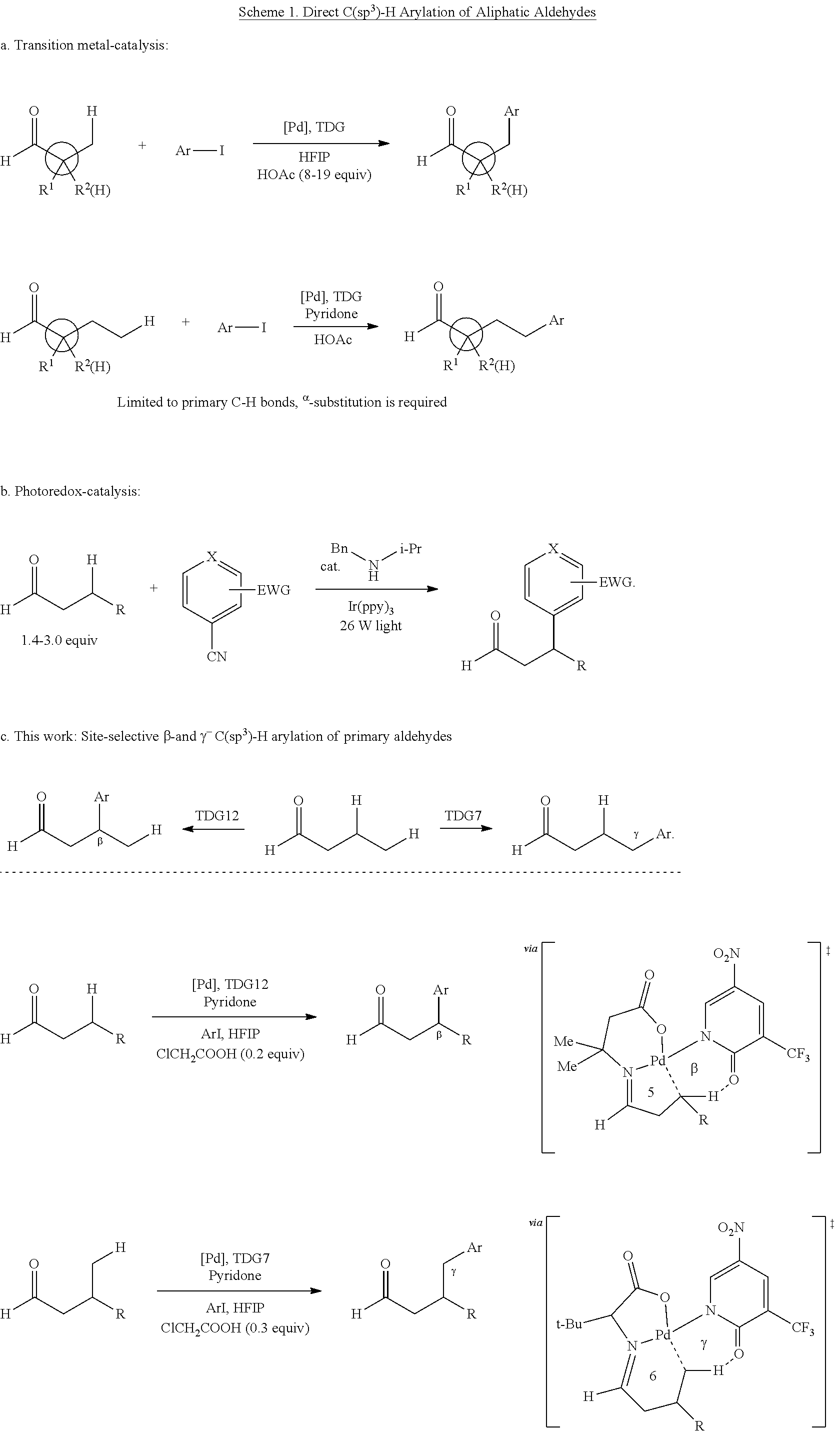

To address the limitation of β- or γ-C—H functionalizations of aldehydes, we began to search for more effective ligands and TDG. Since the discovery of 2-pyridones as effective ligands for non-directed C—H activation of arenes,⁹ this class of ligands has also found applications in several TDG-mediated sp³ and sp² C—H activation reactions.¹⁰ However, for TDG-mediated reactions, large excess of carboxylic acid is usually required to catalyze the attachment and dissociation of TDG, hence, the carboxylate could compete with pyridone for coordination and reduce the ligand acceleration effect (Scheme 2).

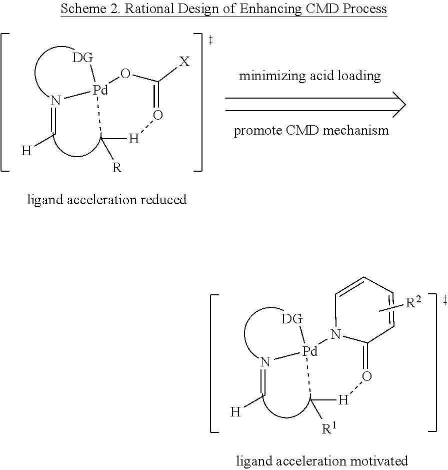

Thus, we began to investigate the influence of the acid loading on the reaction.

In the mixture of HOAc/HFIP (1/5, v/v), model substrate decanal Pd(OAc)₂ (10 mol %), 3-amino-3-methylbutyric acid (TDG12, 30 mol %), 5-nitro-3-(trifluoromethyl)-2-pyridone (L8, 80 mol %), AgTFA (1.5 equiv) and Ag₂CO₃ (0.5 equiv) at 110° C. for 26 h. The reaction mixture was filtered through a short celite pad, followed by solvent removal to afford the β-C(sp³)—H arylation product 2a in 45% NMR yield (Table 1, entry 1). By lowering the acid loading to 5.7 equiv, the reaction mass balance improved significantly from 52% to 72%. When minimal amount (0.2 equiv) of acid was used, 54% desired product was observed with mass balance reaching its highest at 92%. This observation is in line with our hypothesis that superstoichiometric amounts of carboxylates prevent ligand accelerated C—H activation and promote side reactions. Several other organic acids with lower pKₐ were tested for their ability to promote the reversible imine formation with lower loading (entries 4-7). To our delight, replacing acetic acid with 0.2 equiv chloroacetic acid was found to be optimal, achieving 80% NMR yield of the product (entry 7). Notably, the reaction could occur without acid albeit with halved yield (entry 8). Presumably, the mild acidity of HFIP could catalyze the imine formation. Different pyridone ligands were also evaluated for this reaction. Among unfunctionalized 2-pyridone (L1, entry 9) and 5-substituted 2-pyridones (L2-L6, entries 10-14), 5-nitro-2-pyridone (L6) gave the highest yield of 41%. Moreover, replacing the trifluoromethyl group (CF₃) at the 3-position of L8 with a methyl or a nitro group (L7 and L9) proved to be inefficient (entries 16-17). Not surprisingly, no arylation product was obtained in the absence of pyridone (entry 17).

TABLE 1
Evaluation of Acids and Ligands^{a,b}

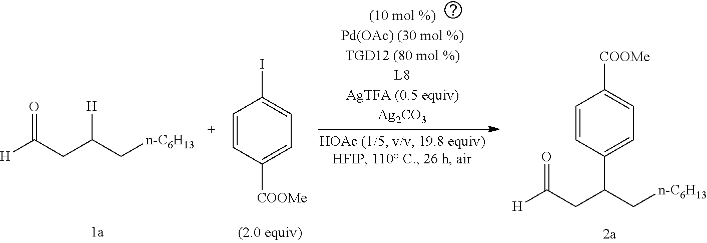

| Entry | Deviation from initial conditions | 2a^{(%)} |
|---|---|---|
| 1 | none | 45 (52)^{d} |
| 2 | HOAc (1/20, v/v, 5.7 equiv) | 62 (72)^{d} |
| 3 | HOAc (0.2 equiv) | 54 (92)^{d} |
| 4 | TFA (0.2 equiv) as acid^{c} | 57 |
| 5 | F₂CHCOOH (0.1 equiv) as acid^{c} | 53 |
| 6 | Cl₃CCOOH (0.1 equiv) as acid^{c} | 36 |
| 7 | ClCH₂COOH (0.2 equiv) as acid^{c} | 80 (96)^{d} |
| 8 | No acid | 46 (96)^{d} |
| 9 | L1 instead of L8, ClCH₂COOH (0.2 equiv) | 8 |
| 10 | L2 instead of L8, ClCH₂COOH (0.2 equiv) | 19 |
| 11 | L3 instead of L8, ClCH₂COOH (0.2 equiv) | 29 |
| 12 | L4 instead of L8, ClCH₂COOH (0.2 equiv) | 36 |
| 13 | L5 instead of L8, ClCH₂COOH (0.2 equiv) | 35 |
| 14 | L6 instead of L8, ClCH₂COOH (0.2 equiv) | 41 |
| 15 | L7 instead of L8, ClCH₂COOH (0.2 equiv) | 41 |
| 16 | L9 instead of L8, ClCH₂COOH (0.2 equiv) | 14 |
| 17 | No Ligand, ClCH₂COOH (0.2 equiv) | n.d. |

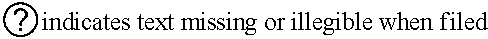

L1 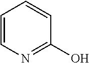
L2 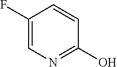
L3 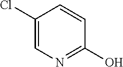
L4 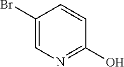
L5 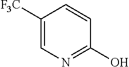 [SMILES: Oc1ccc(C(F)(F)F)cn1]
L6 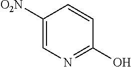
L7 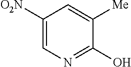 [SMILES: Cc1cc([N+](=O)[O-])cnc1O]
L8 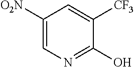 [SMILES: O=[N+]([O-])c1cnc(O)c(C(F)(F)F)c1]
L9 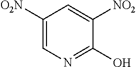 [SMILES: O=[N+]([O-])c1cnc(O)c([N+](=O)[O-])c1]
^{a}Conditions: 1a (0.1 mmol, 1.0 equiv), methyl 4-iodobenzoate (2.0 equiv), Pd(OAc)₂ (10 mol %), TDG12 (30
mol %), ligand (80 mol %), AgTFA (1.5 equiv), Ag₂CO₃ (0.5 equiv) and acid in HFIP (0.75 mL), 110° C., under
air, 26 h.
^{b}Yield determined by ¹H NMR; CH₂Br₂ as internal standard.
^{c}Loading that gave the highest yield within a serial of concentrations.
See SI for detailed screening.
^{d}Mass balance (combined yields of product and unreacted starting material).

With the optimized conditions in hand, a variety of primary aldehydes with methylene β-C(sp³)—H bonds were tested using methyl 4-iodobenzoate as the coupling partner (Table 2). Linear aldehydes were functionalized at the β-position to furnish 2a-2c with good yields. Aldehyde bearing a large cyclohexyl group at the γ-position showed inferior reactivity (2d, 41% yield), while cyclohexyl at the y-position did not inhibit the reaction (2e). Arylation of benzylic β-C(sp³)—H was also compatible, providing 2f in a moderate yield. Substrates containing phenyl, fluoro, amide, acetate, ether, and N-oxyamide groups could all be functionalized with moderate to good yields (2g-2o). The reaction could be readily carried out in gram scale to provide 2a in 70% yield (1.22 g isolated).

TABLE 2
Scope of Aldehydes for β-C(sp³)—H Arylation^{a,b}

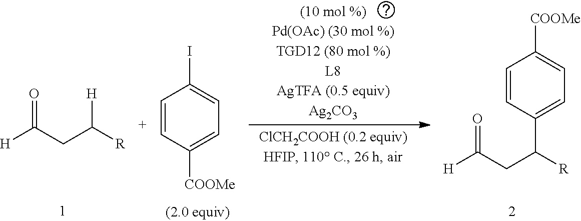

| 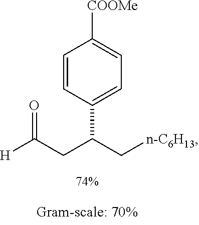 | 2a |
|---|---|
| 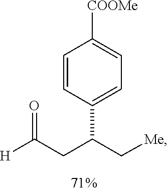 [SMILES: CC[C@H](CC=O)c1ccc(C(=O)OC)cc1] | 2b |
| 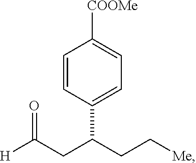 [SMILES: CCC[C@H](CC=O)c1ccc(C(=O)OC)cc1] | 2c |
| 70% | |
| 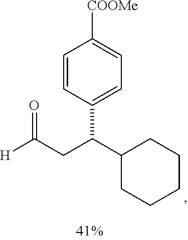 [SMILES: COC(=O)c1ccc([C@@H](CC=O)C2CCCCC2)cc1] | 2d |
| 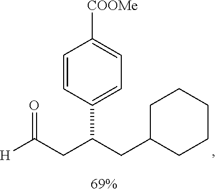 [SMILES: COC(=O)c1ccc([C@@H](CC=O)CC2CCCCC2)cc1] | 2e |
| 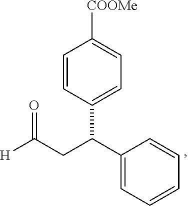 [SMILES: COC(=O)c1ccc([C@@H](CC=O)c2ccccc2)cc1] | 2f |
| 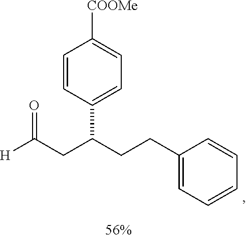 [SMILES: COC(=O)c1ccc([C@@H](CC=O)CCc2ccccc2)cc1] | 2g |
| 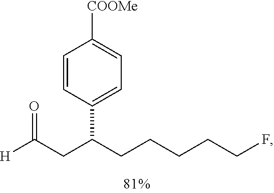 [SMILES: COC(=O)c1ccc([C@@H](CC=O)CCCCCF)cc1] | 2h |
| 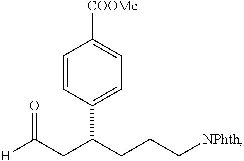 | 2i |
| 62%^{c} | |
| 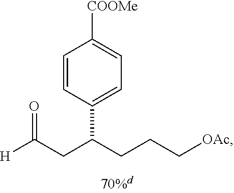 [SMILES: COC(=O)c1ccc([C@@H](CC=O)CCCOC(C)=O)cc1] | 2j |
| 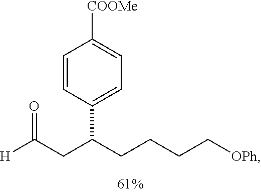 [SMILES: COC(=O)c1ccc([C@@H](CC=O)CCCCOc2ccccc2)cc1] | 2k |
| 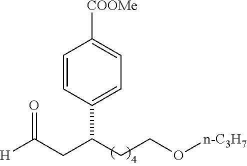 | 2l |
| 69% | |
| 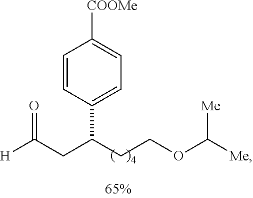 | 2m |
| 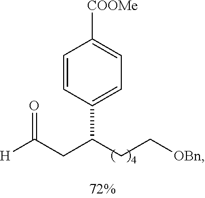 | 2n |
| 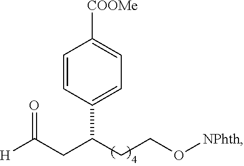 | 2o |
| 71% | |

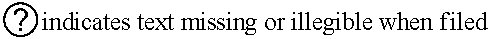

^{a}Conditions: 1 (0.1 mmol, 1.0 equiv), methyl 4-iodobenzoate (2.0 equiv), Pd(OAc)₂ (10 mol %), TDG12 (30
mol %), L8 (80 mol %), AgTFA (1.5 equiv), Ag₂CO₃ (0.5 equiv) and ClCH₂COOH (0.2 equiv) in HFIP (0.75 mL),
110° C., under air, 26 h.
^{b}Isolated yields.
^{c}Reaction time 32 h.
^{d}Reaction time 72 h.

Synthetic versatility of this reaction was further explored with the aryl iodide scope (Table 3). We selected the high boiling point decanal (1a) as the model substrate in order to readily determine mass balance and reaction time. The reaction was compatible with a broad scope of aryl iodides. Good to relatively high yields were acquired with para-substituted electron-deficient aryl iodides containing halogen, acetyl, trifluoromethyl and nitro groups (3a-3f). Surprisingly, the reaction also tolerated unprotected carboxylic acid functionality in the coupling partner to give 3g in 57% yield with longer reaction time. However, the reaction with para-cyano-substituted aryl iodide resulted in 3h with only 45% yield. The reactivities of electron-neutral iodides and iodides with election-donating groups were slightly lower, providing 3i-3k in good to moderate yields. For other aryl iodides containing a meta-ester, nitro, and trifluoromethoxy group, good yields were also obtained (3l-3n). The ortho-fluoro-substituted aryl iodide showed moderate reactivity due to steric hindrance (3o).

TABLE 3
Scope of Aryl Iodide for β-C(sp³)—H Arylation^{a,b}

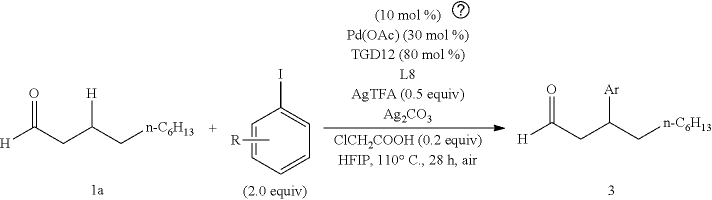

| 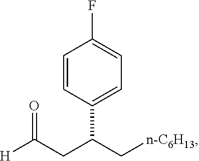 | 3a |
|---|---|
| 80% | |
| 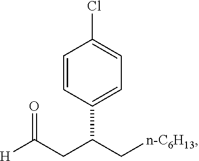 | 3b |
| 69% | |
| 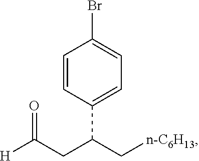 | 3c |
| 70% | |
| 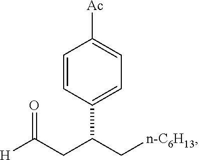 | 3d |
| 69% | |
| 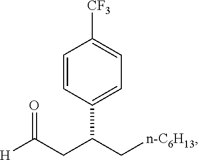 | 3e |
| 71%^{c} | |
| 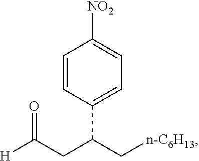 | 3f |
| 83% | |
| 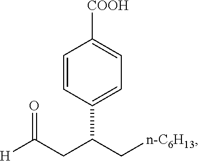 | 3g |
| 57%^{d} | |
| 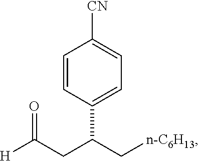 | 3h |
| 45% | |
| 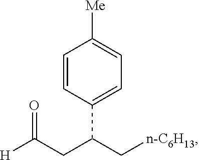 | 3i |
| 60% | |
| 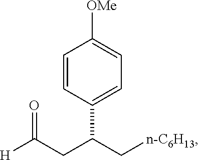 | 3j |
| 52% | |
| 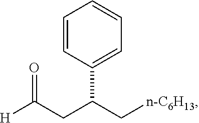 | 3k |
| 55% | |
| 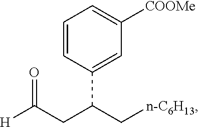 | 3l |
| 72% | |
| 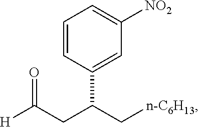 | 3m |
| 78% | |
| 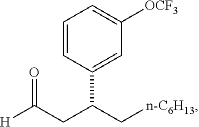 | 3n |
| 63% | |
| 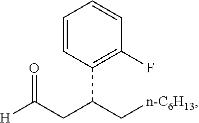 | 3o |
| 58% | |

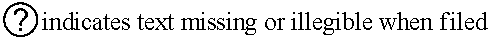

^{a}Conditions: 1a (0.1 mmol, 1.0 equiv), aryl iodide (2.0 equiv), Pd(OAc)₂ (10 mol %), TDG12 (30 mol %), L8 (80 mol %), AgTFA
(1.5 equiv), Ag₂CO₃ (0.5 equiv) and ClCH₂COOH (0.2 equiv) in HFIP (0.75 mL), 110° C., under air, 28 h.
^{b}Isolated yields.
^{c}Reaction time 24 h.
^{d}Reaction time 36 h.

When this-methylene C—H arylation protocol was extended to γ-C—H arylation of aldehyde 4a, less than 10% of the desired product was obtained. We wondered whether the six-membered cyclopalladation of the γ-C—H bond could be promoted by a TDG chelating with Pd (II) via 5-membered ring due to a better match of the bite angles. To our delight, tert-Leucine (TDG7) efficiently directed γ-C(sp³)—H arylation of 3-methylbutanal, forming mono-and di-arylated products in 72% combined yield under slightly modified conditions. We then investigated other primary aldehydes to demonstrate the scope of compatible substrates (Table 4). The protocol tolerated a moderate to bulky substitution at the β-position. Substrates with pentyl, neopentyl, 4-methylpentyl, cyclohexyl or cyclopentyl groups could be transformed to the corresponding products in good yields (5b-5f). Acetoxy and phenyl groups were also shown to be compatible (5g-5h). Other primary aldehydes containing β-quaternary centers could be arylated at the γ-position efficiently, achieving good to moderate yields (5i-5k). At this stage, methylene γ-C(sp³)—H arylation is less efficient, affording low yield (5l).

TABLE 4
Scope of Aldehydes for γ-C(sp³)—H Arylation^{a,b}

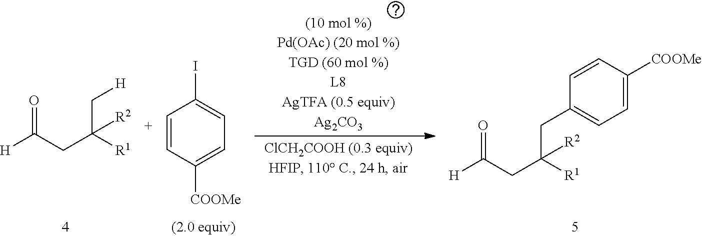

| 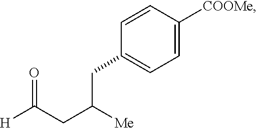 | 5a |
|---|---|
| 72% (40:32)^{c} | |
| 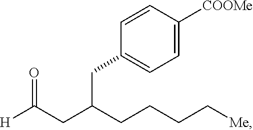 | 5b |
| 60% | |
| 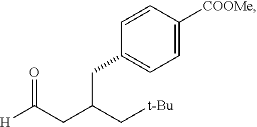 | 5c |
| 72%^{d} | |
| 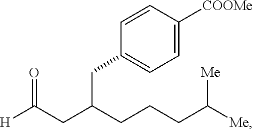 | 5d |
| 61% | |
| 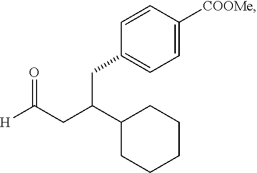 | 5e |
| 60% | |
| 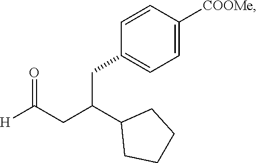 | 5f |
| 59% | |
| 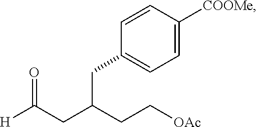 | 5g |
| 56% | |
| 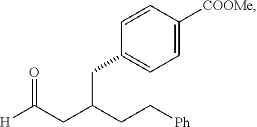 | 5h |
| 45% | |
| 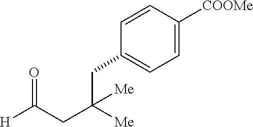 | 5i |
| 83% (60:23)^{c} | |
| 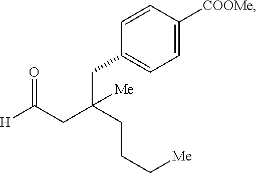 | 5j |
| 60% (44:16)^{c} | |
| 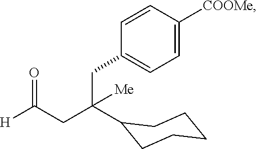 | 5k |
| 46% (37:9)^{c} | |
| 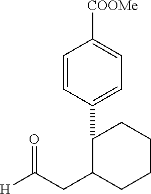 [SMILES: COC(=O)c1ccc([C@@H]2CCCCC2CC=O)cc1] | 5l |
| 20% | |

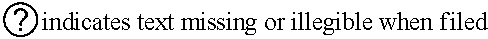

^{a}Conditions: 4 (0.1 mmol, 1.0 equiv), methyl 4-iodobenzoate (2.0 equiv), Pd(OAc)₂ (10 mol %), TDG7 (20 mol %), L8 (60 mol %),
AgTFA (1.5 equiv), Ag₂CO₃ (0.5 equiv) and ClCH₂COOH (0.3 equiv) in HFIP (0.50 mL), 110° C., under air, 24 h.
^{b}Isolated yields.
^{c}Ratio of mono: di.
^{d}Reaction time 36 h.

γ-C—H arylation reaction of 4c with a plethora of aryl iodides exhibited a good functional-group compatibility (Table 5). Aryl iodides with various electron-withdrawing groups at the para, meta or ortho positions were coupled to the desired γ-C(sp³)—H bonds in good yields (6a-6f). Coordinating groups such as nitro, acetyl, and cyano were also compatible (6g-6j). In addition, the reaction of fluorescent para-substituted N-(p-iodophenyl)-1,8-naphthalimide resulted in the fluorophore conjugate 6k with 56% yield. Furthermore, aryl iodides derived from natural products such as estrone and borneol were also effectively functionalized to afford the desired products in 65% and 57% yields, respectively (6l-6m). However, electron-neutral and electron-rich aryl iodides exhibited poor reactivity, with 10-20% yields observed using 4-iodotoluene and 4-iodoanisole as coupling partners, for instance.

TABLE 5
Scope of Aryl Iodide for γ-C(sp³)—H Arylation^{a,b}

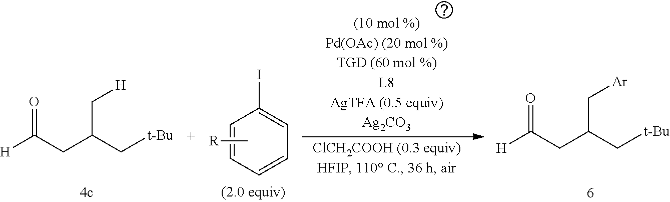

| 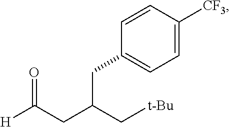 | 6a |
|---|---|
| 64% | |
| 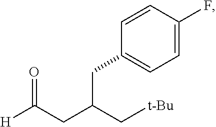 | 6b |
| 57% | |
| 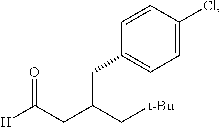 | 6c |
| 64% | |
| 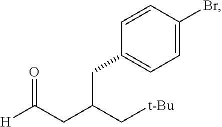 | 6d |
| 63% | |
| 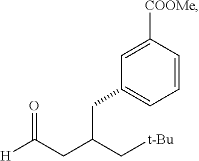 | 6e |
| 69% | |
| 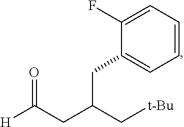 | 6f |
| 61% | |
| 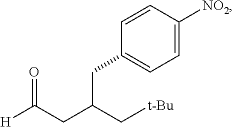 | 6g |
| 70% | |
| 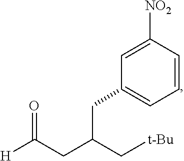 | 6h |
| 71% | |
| 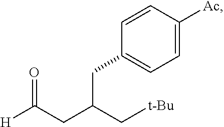 | 6i |
| 59% | |
| 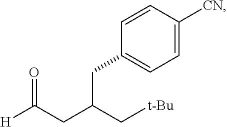 | 6j |
| 48% | |
| 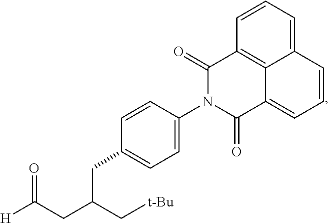 | 6k |
| 56% | |
| 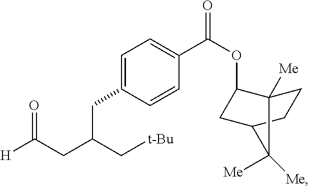 | 6l |
| 65% | |
| 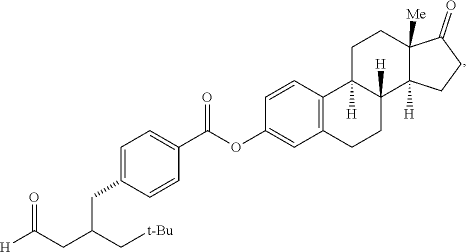 | 6m |
| 57% | |

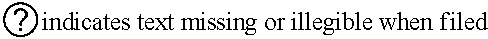

^{a}Conditions: 4c (0.1 mmol, 1.0 equiv), aryl iodide (2.0 equiv), Pd(OAc)₂ (10 mol %), TDG7 (20 mol %), L8 (60 mol %),
AgTFA (1.5 equiv), Ag₂CO₃ (0.5 equiv) and ClCH₂COOH (0.3 equiv) in HFIP (0.50 mL), 110° C., under air, 36 h.
^{b}Isolated yields.

To further illustrate the impact of the chelating ring size of TDG on site-selectivity, we attempted the challenging site-selective C(sp³)—H activation of a representative substrate containing both β-methylene and γ-primary C—H bonds (Scheme 3). To our delight, arylation of butanal 1p afforded over 77% yield of the desired product (2p) with an exclusive β-selectivity (β/γ>20:1) when 6-membered chelating TDG (TDG12) was used. In contrast, the selectivity was switched to γ-arylation (γ/β=9:1) in 62% yield with 5-membered chelating TDG (TDG7). Considering that previously reported C(sp³)—H activation reactions via a 6-membered palladacycle intermediate often needed substitutions at α/β positions to prevent β-C(sp³)—H activation, the impact of the TDG on site-selectivity is significant.¹¹

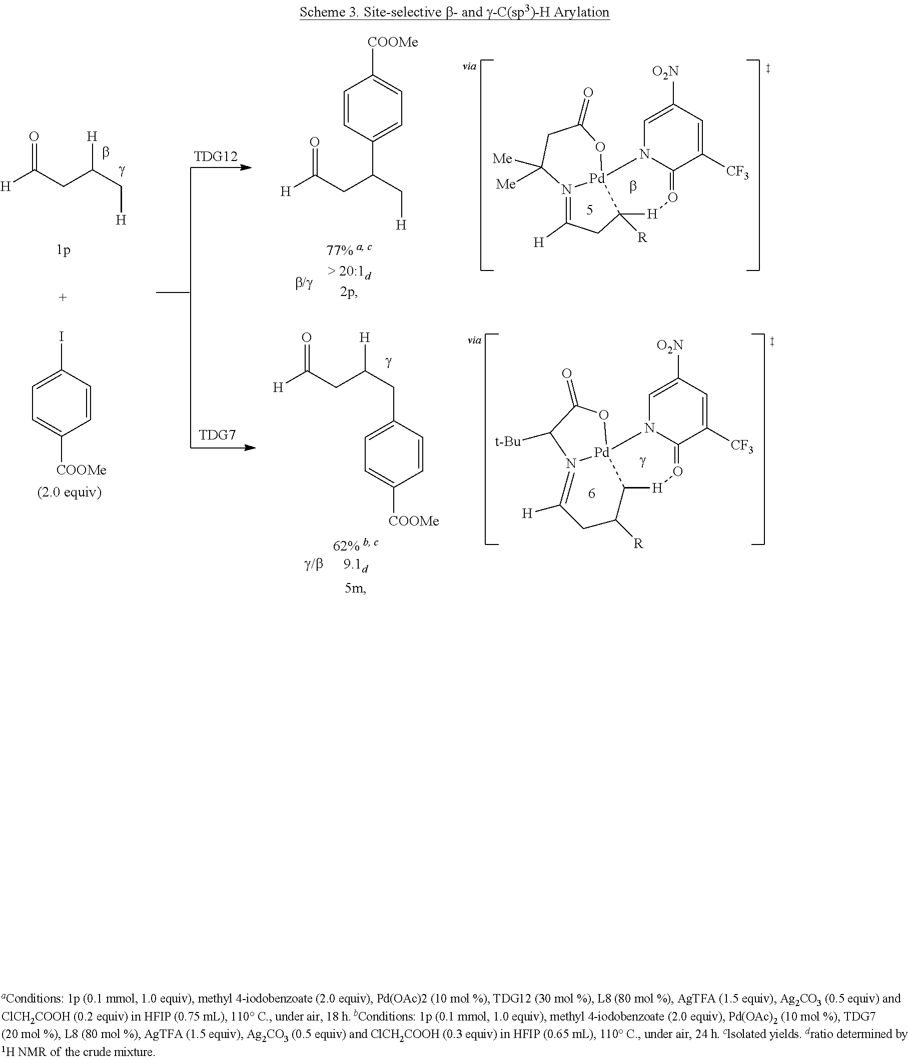

Although we have reported a single example of controlling γ/β selectivity in directed C(sp³)—H arylation of alcohols by designing different covalent L,X-type directing groups, the origin of the selectivity has not been investigated in-depth.^{12,13} The β-site-selectivity using amino acid-based TDG for C(sp³)—H activation of ketones has been rationalized through computational studies.¹⁴ This first example of TDG-controlled β- and γ-C(sp³)—H activation offers us a unique opportunity to probe the origin of site-selectivity. We hence performed deuterium incorporation experiments in the presence of 2-chloroacetic acid-d and HFIP-ol-D (Scheme 4a). The absence of deuterium incorporation in the arylated products suggested that the C—H cleavage step was irreversible for both β- and γ-C(sp³)—H arylation. Moreover, kinetic isotope effect (KIE) studies revealed large primary KIE values (KIE_{β} of 7.8 and KIE_{γ} of 5.6) when using β- and y-deuterated substrates (Scheme 4b). These results are consistent with the C—H cleavage being the rate-and site-selectivity-determining step for both β- and γ-C(sp³)—H arylation.

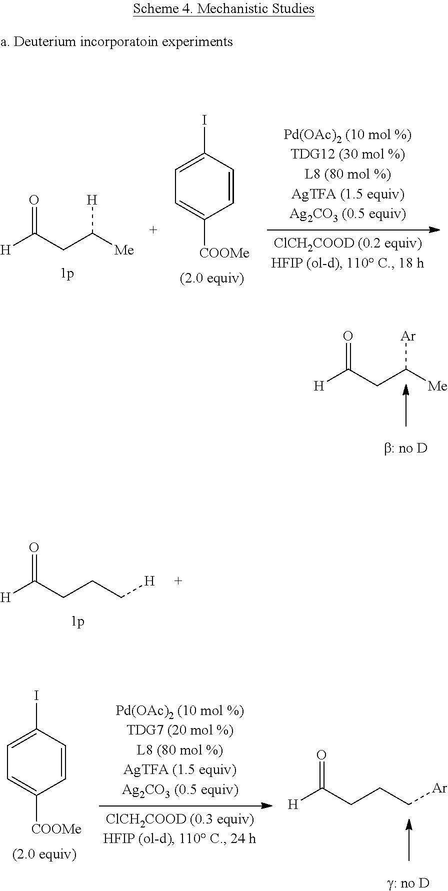

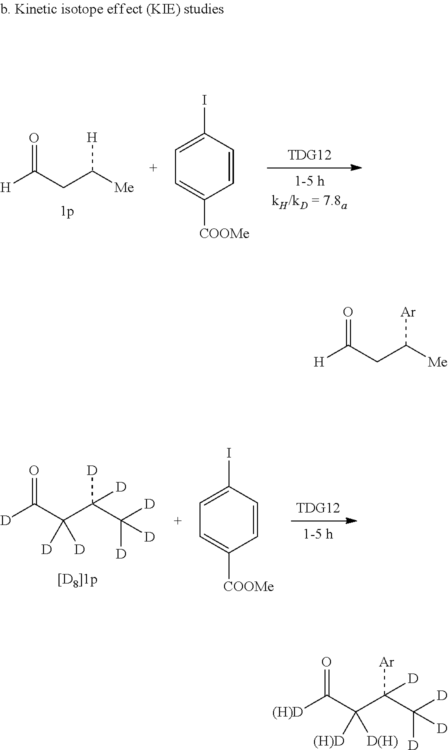

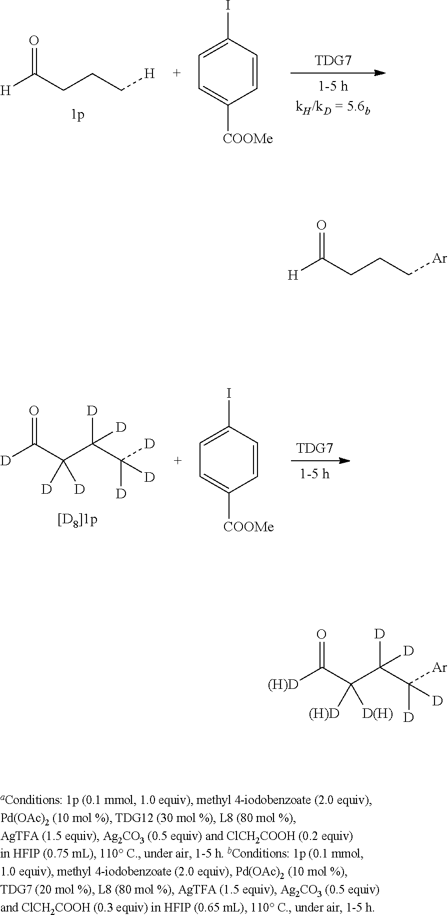

With these findings in hand, we began to investigate the influence of TDG on site-selectivity by DFT modeling of the corresponding C—H cleavage transition states (TS). We used 1p as the model substrate for our studies (please see the SI for computational details). With L8 as the ligand, 4 ensembles of TS were located, corresponding to β- and γ-C(sp³)—H activation with TDG12 and TDG7 (Scheme 5, lowest TS shown).

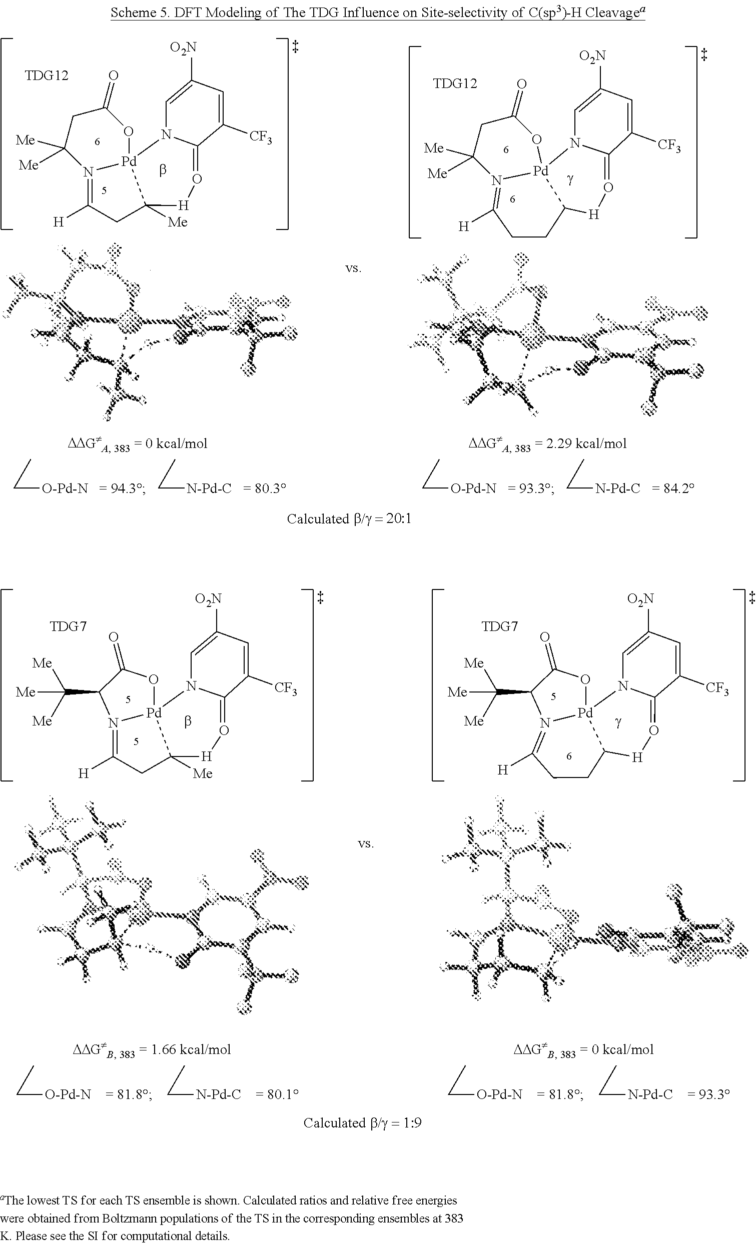

Calculated β/γ site-selectivity and relative activation free energies (ΔΔG 383) were obtained from the ratios of combined Boltzmann populations of the corresponding TS ensembles. β-C(sp³)—H activation was calculated to be favored over γ- by 2.29 kcal/mol with TDG12, while with TDG7 the selectivity was reversed with a free energy difference of 1.66 kcal/mol. These values correspond to 20:1 and 1:9 calculated β/γ- ratios, respectively, in excellent agreement with the experimental observations (Scheme 3). Our studies suggest that the site-selectivity of C—H cleavage is controlled by the bite angle of the TDG. Evidently, the 5,6-membered coordination (with O—Pd—C angle of around) 175° is preferred over the 5,5-or 6,6-membered coordination, where additional ring strain in the C—H cleavage TS renders them less favored. This finding is also consistent with the ring strain of fused carbocyclic rings.¹⁵

In summary, we have developed a protocol for Pdll-catalyzed site-selective β-methylene and γ-C(sp³)—H arylation of primary aldehydes. This reaction features broad substrate scope with good functional group compatibility, exemplified by successful C—H arylation of a range of readily oxidizable aldehydes under mild conditions. Moreover, this strategy highlighted the influence of TDG on site-selectivity through matching the bite angles between the palladacycle and the TDG chelation.

### REFERENCES

(1) Zhang, F.-L.; Hong, K.; Li, T.-J.; Park, H.; Yu, J.-Q. Functionalization of C(sp³)—H Bonds Using a Transient Directing Group. Science 2016, 351, 252-256.

(2) For selected aliphatic ketone C(sp³)—H arylations, see: (a) K. Hong; H. Park; J.-Q. Yu. Methylene C(sp³)—H Arylation of Aliphatic Ketones Using a Transient Directing Group. ACS Catal. 2017, 7, 6938-6941. (b) L. Pan; K. Yang; G. Li; H. Ge. Palladium-catalyzed site-selective arylation of aliphatic ketones enabled by a transient ligand. Chem. Commun. 2018, 54, 2759-2762. (c) Wang, J.; Dong, C.; Wu, L.; Xu, M.; Lin, J.; Wei, K. Palladium-Catalyzed β-C—H Arylation of Ketones Using Amino Amide as a Transient Directing Group: Applications to Synthesis of Phenanthridinone Alkaloids. Adv. Synth. Catal. 2018, 360, 3709-3715. (d) L.-J. Xiao; K. Hong; F. Luo; L. Hu; W. R. Ewing; K.-S. Yeung; J.-Q. Yu. Pd^{II}-Catalyzed Enantioselective C(sp³)—H Arylation of Cyclobutyl Ketones Using a Chiral Transient Directing Group. Angew. Chem., Int. Ed. 2020, 59, 9594-9600. (e) Provencher, P. A.; Bay, K. L.; Hoskin, J. F.; Houk, K. N.; Yu, J.-Q.; Sorensen, E. J. Cyclization by C(sp³)—H Arylation with a Transient Directing Group for the Diastereoselective Preparation of Indanes. ACS Catal. 2021, 11, 3115-3127. (f) Provencher, P. A.; Hoskin, J. F.; Wong, J. J.; Chen, X.; Yu, J.-Q.; Houk, K. N.; Sorensen, E. J. Pd (II)-Catalyzed Synthesis of Benzocyclobutenes by β-Methylene-Selective C(sp³)—H Arylation with a Transient Directing Group. J. Am. Chem. Soc. 2021. doi: 10.1021/jacs. 1c09368.

(3) For selected aliphatic amine C(sp³)—H functionalization, see: (a) Y. Xu; M. C. Young; C. Wang; D. M. Magness; G. Dong. Catalytic C(sp³)—H Arylation of Free Primary Amines with an exo Directing Group Generated In Situ. Angew. Chem., Int. Ed. 2016, 55, 9084-9087. (b) Wu, Y.; Chen, Y.-Q.; Liu, T.; Eastgate, M. D.; Yu, J.-Q. Pd-Catalyzed γ-C(sp3)-H Arylation of Free Amines Using a Transient Directing Group. J. Am. Chem. Soc. 2016, 138, 14554-14557. (c) Y. Liu; H. Ge. Site-selective C—H arylation of primary aliphatic amines enabled by a catalytic transient directing group. Nat. Chem. 2016, 9, 26-32. (d) Chen, Y.-Q.; Wang, Z.; Wu, Y.; Wisniewski, S. R.; Qiao, J. X.; Ewing, W. R.; Eastgate, M. D.; Yu, J.-Q. Overcoming the Limitations of γ- and δ-C—H Arylation of Amines through Ligand Development. J. Am. Chem. Soc. 2018, 140, 17884-17894. (e) Chen, Y. Q.; Wu, Y. W.; Wang, Z.; Qiao, J. X.; Yu, J. Q. Transient Directing Group Enabled Pd-Catalyzed Gamma-C(sp³)—H Oxygenation of Alkyl Amines. ACS Catal. 2020, 10, 5657-5662. (f) Chen, Y.-Q.; Singh, S.; Wu, Y.; Wang, Z.; Hao, W.; Verma, P.; Qiao, J. X.; Sunoj, R. B.; Yu, J.-Q. Pd-Catalyzed γ-C(sp³)—H Fluorination of Free Amines. J. Am. Chem. Soc. 2020, 142, 9966-9974.

(4) For selected benzylic C(sp³)—H functionalization of o-tolualdehydes using transient directing groups, see: (a) Zhang, F.-L.; Hong, K.; Li, T.-J.; Park, H.; Yu, J.-Q. Functionalization of C(sp³)—H Bonds Using a Transient Directing Group. Science 2016, 351, 252-256. (b) Ma, F.; Lei, M.; Hu, L. Acetohydrazone: A Transient Directing Group for Arylation of Unactivated C(sp³)—H Bonds. Org. Lett. 2016, 18, 2708-2711. (c) Park, H.; Verma, P.; Hong, K.; Yu, J.-Q. Controlling Pd (IV) Reductive Elimination Pathways Enables Pd(II)-Catalysed Enantioselective C(sp³)—H Fluorination. Nat. Chem. 2018, 10, 755-762. (d) Tang, M.; Yu, Q.; Wang, Z.; Zhang, C.; Sun, B.; Yi, Y.; Zhang, F.-L. Synthesis of Polycyclic Aromatic Hydrocarbons (PAHs) via a Transient Directing Group. Org. Lett. 2018, 20, 7620-7623. (e) Park, H.; Yoo, K.; Jung, B.; Kim, M. Direct Synthesis of Anthracenes from o-Tolualdehydes and Aryl Iodides through Pd(II)-Catalyzed sp3 C—H Arylation and Electrophilic Aromatic Cyclization. Tetrahedron 2018, 74, 2048-2055. (f) Wang, Z.; Dong, W.; Sun, B.; Yu, Q.; Zhang, F.-L. Cascade Reaction for the Synthesis of Polycyclic Aromatic Hydrocarbons via Transient Directing Group Strategy. Tetrahedron 2019, 75, 4031-4041. (g) Ding, M.; Hua, W.; Liu, M.; Zhang, F. Pd-Catalyzed C(sp³)—H Biarylation via Transient Directing Group Strategy. Org. Lett. 2020, 22, 7419-7423. (h) Bai, C.; Chao, B.; Muschin, T.; Bao, A.; Baiyin, M.; Liu, D.; Bao, Y.-S. Regiodivergent CDC reactions of aromatic aldehydes with unactivated arenes controlled by transient directing strategy. Chem. Commun. 2021, 57, 11229-11232.

(5) For selected C(sp²)—H functionalization of benzaldehydes using transient directing groups, see: (a) Liu, X.-H.; Park, H.; Hu, J.-H.; Hu, Y.; Zhang, Q.-L.; Wang, B.-L.; Sun, B.; Yeung, K.-S.; Zhang, F.-L.; Yu, J.-Q. Diverse Ortho-C(sp²)—H Functionalization of Benzaldehydes Using Transient Directing Groups. J. Am. Chem. Soc. 2017, 139, 888-896. (b) Chen, X.-Y.; Ozturk, S.; Sorensen, E. J. Synthesis of Fluorenones from Benzaldehydes and Aryl Iodides: Dual C—H Functionalizations Using a Transient Directing Group. Org. Lett. 2017, 19, 1140-1143. (c) Chen, X.-Y.; Ozturk, S.; Sorensen, E. J. Pd-Catalyzed Ortho C—H Hydroxylation of Benzaldehydes Using a Transient Directing Group. Org. Lett. 2017, 19, 6280-6283. (d) Chen, X.-Y.; Sorensen, E. J. Pd-Catalyzed, Ortho C—H Methylation and Fluorination of Benzaldehydes Using Orthanilic Acids as Transient Directing Groups. J. Am. Chem. Soc. 2018, 140, 2789-2792. (e) Li, F.; Zhou, Y.; Yang, H.; Liu, D.; Sun, B.; Zhang, F.-L. Assembly of Diverse Spirocyclic Pyrrolidines via Transient Directing Group Enabled Ortho-C(sp²)—H Alkylation of Benzaldehydes. Org. Lett. 2018, 20, 146-149. (f) Li, B.; Seth, K.; Niu, B.; Pan, L.; Yang, H.; Ge, H. Transient-Ligand-Enabled ortho-Arylation of Five-Membered Heterocycles: Facile Access to Mechanochromic Materials. Angew. Chem., Int. Ed. 2018, 57, 3401-3405. (g) Qiao, H.; Sun, B.; Yu, Q.; Huang, Y.; Zhou, Y.; Zhang, F. Palladium-Catalyzed Direct Ortho-C—H Selenylation of Benzaldehydes Using Benzidine as a Transient Directing Group. Org. Lett. 2019, 21, 6914-6918. (h) Li, F.; Zhou, Y.; Yang, H.; Wang, Z.; Yu, Q.; Zhang, F.-L., Monodentate Transient Directing Group Enabled Pd-Catalyzed Ortho-C—H Methoxylation and Chlorination of Benzaldehydes. Org. Lett. 2019, 21, 3692-3695.

(6) For selected β- and γ-C(sp³)—H functionalization of aldehydes using transient directing groups, see: (a) Yang, K.; Li, Q.; Liu, Y.; Li, G.; Ge, H. Catalytic C—H Arylation of Aliphatic Aldehydes Enabled by a Transient Ligand. J. Am. Chem. Soc. 2016, 138, 12775-12778. (b) St John-Campbell, S.; White, A. J. P.; Bull, J. A. Single Operation Palladium Catalysed C(sp³)—H Functionalisation of Tertiary Aldehydes: Investigations into Transient Imine Directing Groups. Chem. Sci. 2017, 8, 4840-4847. (c) Dong, C.; Wu, L.; Yao, J.; Wei, K. Palladium-Catalyzed β-C—H Arylation of Aliphatic Aldehydes and Ketones Using Amino Amide as a Transient Directing Group. Org. Lett. 2019, 21, 2085-2089. (d) St John-Campbell, S.; Bull, J. A. Intramolecular Palladium(II/IV) Catalysed C(sp³)—H Arylation of Tertiary Aldehydes Using a Transient Imine Directing Group. Chem. Commun. 2019, 55, 9172-9175. (e) Gou, B. B.; Yang, H.; Sun, H. R.; Chen, J.; Wu, J.; Zhou, L. Palladium-Catalyzed Site-Selective C(sp³)—H Arylation of Phenylacetaldehydes. Org. Lett. 2019, 21, 80-84. (f) Li, B.; Lawrence, B.; Li, G.; Ge, H. Ligand-Controlled Direct y-C—H Arylation of Aldehydes. Angew. Chem., Int. Ed. 2020, 59, 3078-3082. (g) St John-Campbell, S.; White, A. J. P.; Bull, J. A. Methylene C(sp³)—H β, β′-Diarylation of Cyclohexanecarbaldehydes Promoted by a Transient Directing Group and Pyridone Ligand. Org. Lett. 2020, 22, 1807-1812. (h) Wu, L.-F.; Yao, J.-W.; Zhang, X; Liu, S.-Y.; Zhuang, Z.-N.; Wei, K. Pd-Catalyzed β-C—H Arylation of Aldehydes and Ketones Based on a Transient Directing Group. Org. Lett. 2021, 23, 6237-6241.

(7) Pirnot, M. T.; Rankic, D. A.; Martin, D. B. C.; MacMillan, D. W. C., Photoredox Activation for the Direct β-Arylation of Ketones and Aldehydes. Science 2013, 339, 1593-1596.

(8) Meng, G.; Lam, N. Y. S.; Lucas, E. L.; Saint-Denis, T. G.; Verma, Site-Selectivity for C—H P.; Chekshin, N.; Yu, J.-Q. Achieving Activation Processes Based on Distance and Geometry: A Carpenter's Approach. J. Am. Chem. Soc. 2020, 142, 10571-10591.

(9) Wang, P.; Farmer, M. E.; Huo, X.; Jain, P.; Shen, P.-X.; Ishoey, M.; Bradner, J. E.; Wisniewski, S. R.; Eastgate, M. D.; Yu, J.-Q. Ligand-Promoted Meta-C—H Arylation of Anilines, Phenols, and Heterocycles. J. Am. Chem. Soc. 2016, 138, 9269-9276.

(10) For pyridone ligands enabled TDG mediated C—H functionalization reactions, see: 2d, e, f, 3d, e, f; 5h; 6f, g. For pyridone ligands enabled non TDG mediated C—H functionalization reactions, see: (a) Wang, P.; Verma, P.; Xia, G.; Shi, J.; Qiao, J. X.; Tao, S.; Cheng, P. T. W.; Poss, M. A.; Farmer, M. E.; Yeung, K. S.; Yu, J.-Q., Ligand-Accelerated Non-Directed C—H Functionalization of Arenes. Nature. 2017, 551, 489-493. (b) Wang, P.; Farmer, M. E.; Yu, J.-Q. Ligand-Promoted meta-C—H Functionalization of Benzylamines. Angew. Chem. Int. Ed. 2017, 56, 5125-5129. (c) Farmer, M. E.; Wang, P.; Shi, H.; Yu, J.-Q., Palladium Catalyzed meta-C—H Functionalization of Masked Aromatic Aldehydes. ACS Catal. 2018, 8, 7362-7367. (d) Zhu, R.-Y.; Li, Z. Q.; Park, H. S.; Senanayake, C. H.; Yu, J.-Q. Ligand-Enabled γ-C(sp³)—H Activation of Ketones. J. Am. Chem. Soc. 2018, 140, 3564-3568. (e) Chen, X. Y.; Wu, Y.; Zhou, J.; Wang, P.; Yu, J.-Q. Synthesis of β-Arylethenesulfonyl Fluoride via Pd-Catalyzed Nondirected C—H Alkenylation. Org. Lett. 2019, 21, 1426-1429. (f) Hill, D. E.; Yu, J.-Q.; Blackmond, D. G. Insights into the Role of Transient Chiral Mediators and Pyridone Ligands in Asymmetric Pd-Catalyzed C—H Functionalization. J. Org. Chem. 2020, 85, 13674-13679. (g) Park, H. S.; Fan, Z.; Zhu, R.-Y.; Yu, J.-Q., Distal γ-C(sp³)—H Olefination of Ketone Derivatives and Free Carboxylic Acids. Angew. Chem. Int. Ed. 2020, 59, 12853-12859. (h) Xia, G.; Zhuang, Z.; Liu, L.-Y.; Schreiber, S. L.; Melillo, B.; Yu, J.-Q. Ligand-Enabled β-Methylene C(sp³)—H Arylation of Masked Aliphatic Alcohols. Angew. Chem. Int. Ed. 2020, 59, 7783-7787. (i) Li, Y.; Zhang, P.; Liu, Y.; Yu, Z.; Shi, B. Remote γ-C(sp³)—H Alkylation of Aliphatic Carboxamides via an Unexpected Regiodetermining Pd Migration Process: Reaction Development and Mechanistic Study. ACS Catal. 2020, 10, 8212-8222. For a computational study on efficacy of pyridone ligands, see: (i) Mandal, N.; Datta, A. Harnessing the Efficacy of 2-Pyridone Ligands for Pd-Catalyzed (β/γ)-C(sp³)—H Activations. J. Org. Chem. 2020, 85, 13228-13238.

(11) For selected C(sp³)—H activation of quaternary carbon centers via a 6-membered palladacycle intermediate, see: (a) Giri, R.; Maugel, N.; Foxman, B. M.; Yu, J.-Q. Dehydrogenation of Inert Alkyl Groups via Remote C—H activation: Converting a Propyl Group into a π-allylic Complex. Organometallics 2008, 27, 1667-1670. (b) Nadres, E. T.; Daugulis, O., Heterocycle Synthesis via Direct C—H/N—H Coupling. J. Am. Chem. Soc. 2012, 134, 7-10. (c) Li, S.; Chen, G.; Feng, C.-G.; Gong, W.; Yu, J.-Q., Ligand-Enabled γ-C—H Olefination and Carbonylation: Construction of β-Quaternary Carbon Centers. J. Am. Chem. Soc. 2014, 136, 5267-5270. (d) Li, S.; Zhu, R.-Y.; Xiao, K.-J.; Yu, J.-Q. Ligand-enabled Arylation of γ-C—H Bonds. Angew. Chem., Int. Ed. 2016, 55, 4317-4321. (e) Liu, L.; Liu, Y.-H.; Shi, B.-F. Synthesis of Amino Acids and Peptides with Bulky Side Chains via Ligand-Enabled Carboxylate Directed γ-C(sp³)—H Arylation. Chem. Sci. 2020, 11, 290-294. (f) Ghosh, K. K.; Uttry, A.; Mondal, A.; Ghiringhelli, F.; Wedi, P.; van Gemmeren, M. Ligand-Enabled γ-C(sp³)—H Olefination of Free Carboxylic Acids. Angew. Chem., Int. Ed. 2020, 59, 12848-12852. Also see 3d, 6f, 10d, g, i.

(12) Xia, G.; Weng, J.; Liu, L.; Verma, P.; Li, Z.; Yu, J.-Q., Reversing Conventional Site-selectivity in C(sp³)—H Bond Activation. Nat. Chem. 2019, 11, 571-577.

(13) Jin, X.; Xu, H.; Zhao, N.; Li, R.; Dang, Y. Origins of unconventional γ site selectivity in palladium-catalyzed C(sp³)—H activation and arylation of aliphatic alcohols. Org. Lett. 2020, 22, 1464-1468.

(14) Liu, W.; Zheng, J.; Liu, Z.; Hu, W.; Wang, X.; Dang, Y. How Does Palladium-Amino Acid Cooperative Catalysis Enable Regio and Stereoselective C(sp³)—H Functionalization in Aldehydes and Ketones? A DFT Mechanistic Study. ACS Catal. 2018, 8, 7698-7709.

(15) Anslyn, E. V.; Dougherty, D. A. Modern Physical Organic Chemistry, University Science Books, Sausalito, 2006.

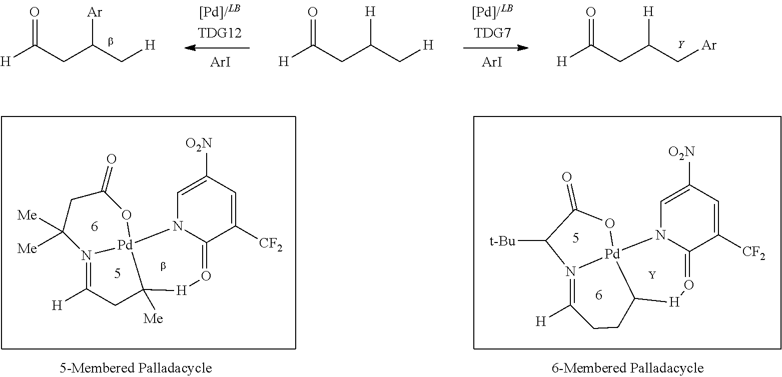

### Synthetic Examples

### Abbreviations

OAc=acetate
Ac=acetyl
TFA=trifluoroacetate
Pd(TFA)₂=Palladium trifluoroacetate
   Materials and methods

### 1. General Information

Substrates were obtained from the commercial sources or synthesized following literature procedures. Solvents were obtained from Sigma-Aldrich, Oakwood and Acros and used directly without further purification. Analytical thin layer chromatography was performed on 0.25 mm silica gel 60-F254. Visualization was carried out with UV light, and Bromocresol Green Stain and Vogel's permanganate. ¹H NMR was recorded on Bruker DRX-600 instrument (600 MHz). Chemical shifts were quoted in parts per million (ppm) referenced to 0.0 ppm for tetramethylsilane. The following abbreviations (or combinations thereof) were used to explain multiplicities: s=singlet, d=doublet, t=triplet, q=quartet, m=multiplet, br=broad. Coupling constants, J, were reported in Hertz unit (Hz). ¹³C NMR spectra were recorded on Bruker DRX-600 instrument (151 MHZ), and were fully decoupled by broad band proton decoupling. ¹⁹F NMR spectra were recorded on Bruker AMX-400 instrument (376 MHZ), and were fully decoupled by broad band proton decoupling. Chemical shifts were reported in ppm referenced to the center line of a triplet at 77.0 ppm of chloroform-d. High-resolution mass spectra (HRMS) were recorded on an Agilent Mass spectrometer using ESI-TOF (electrospray ionization-time of flight).

### 2. Substrate Structures

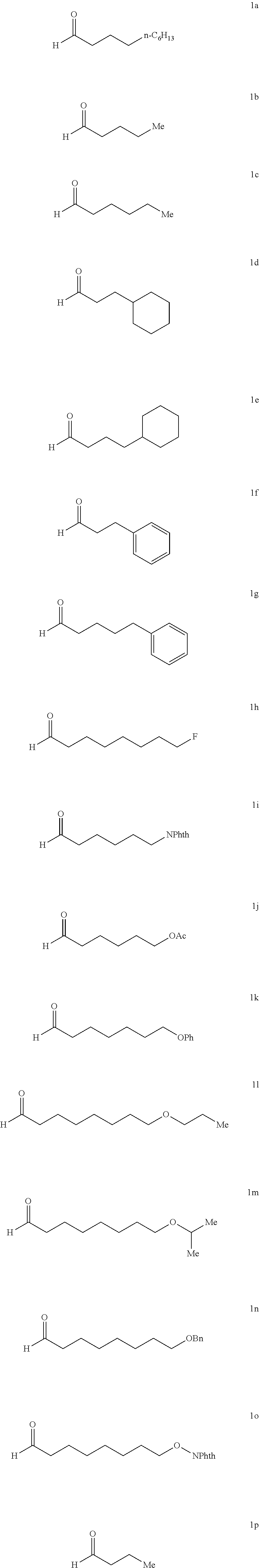

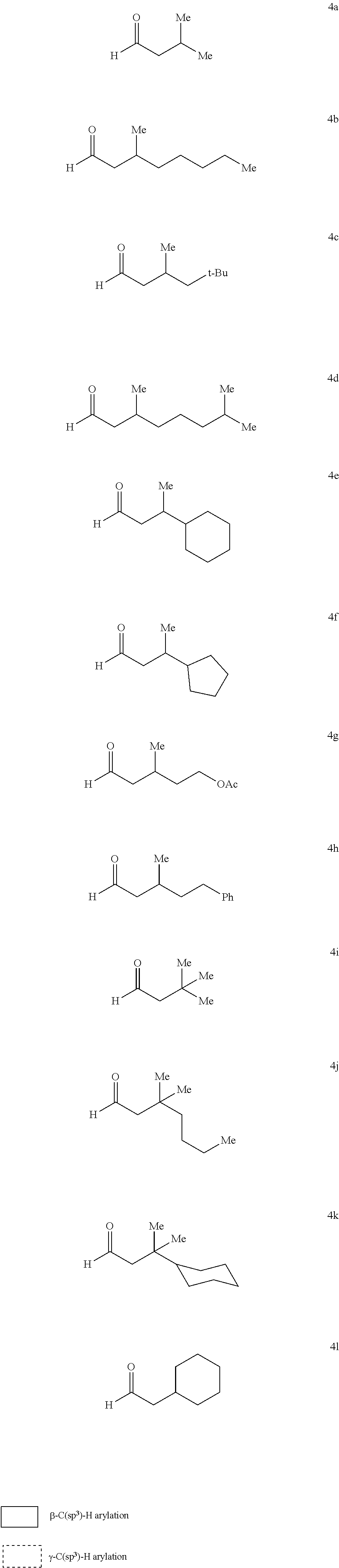

### 3. Substrate Preparation

3.1 Substrates 1a, 1b, 1c, 1p, 4a, 4c and 4i are commercially available and used after distillation.

3.2 Substrates 1d-o, 4d and 4g were synthesized from the corresponding alcohol with the following procedure:

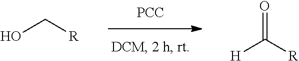

Alcohol (5.0 mmol) was dissolved in DCM (20 mL), cone. Pyridinium chlorochromate (PCC, 7.5 mmol, 1.62 g) added in proportion with stirring and the reaction mixture was stirred at room temperature for 2 h before filtered through a pad of silica gel and concentrated under reduced pressure. The crude aldehyde was then purified by chromatography to give the title compounds. The corresponding alcohol for substrates 1d-g and 4d are commercially available.

The corresponding alcohol for substrates 1h were prepared according to literature procedures^{1,2}.

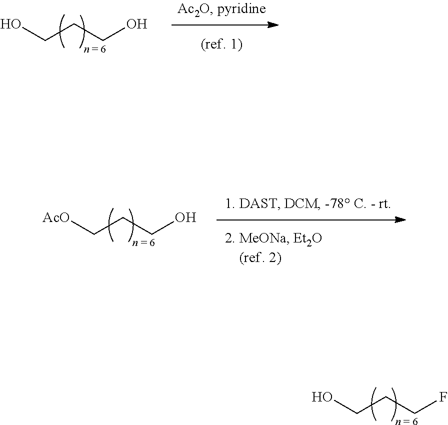

The corresponding alcohol for substrates 1i were prepared according to literature procedures³. The corresponding alcohol for substrates 1j and 4g were prepared according to literature procedures¹.

The corresponding alcohol for substrates 1k were prepared according to literature procedures⁴. The corresponding alcohol for substrates 1l-n were prepared according to literature procedures⁵.

The corresponding alcohol for substrates 10 were prepared according to literature procedures^{6,7}

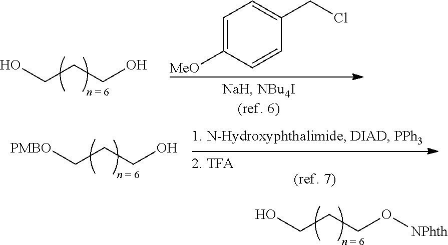

3.3 Substrates 4b, 4e-f and 4h were synthesized from the corresponding carboxylic acid, 4j-k were synthesized form the corresponding ethyl ester with the following procedure:

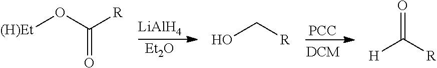

To a stirring suspension of LiAlH₄ (6.5 mmol) in 19 mL Et₂O at 0° C. was added a solution of ester/acid (5 mmol) in 4 mL Et₂O dropwise. The reaction was stirred for 5 min before being brough to reflux for 1 h. The mixture was cooled to 0° C. and 0.3 mL water was carefully added dropwise followed by 0.3 mL 6M NaOH solution, and MgSO₄ sufficient to sequester excess water. The slurry was stirred for 30-60 min and filter through celite. The filtrate was concentrated to give corresponding alcohol. The alcohol was dissolved in DCM (20 mL), cone. Pyridinium chlorochromate (PCC, 7.5 mmol, 1.62 g) added in proportion with stirring and the reaction mixture was stirred at room temperature for 2 h before filtered through a pad of silica gal and concentrated under reduced pressure. The crude aldehyde was then purified by chromatography to give the title compounds.

The corresponding carboxylic acid for substrates 4b, 4e-f and 4h were prepared according to literature procedures^{8,9}.

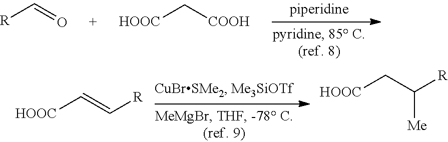

The corresponding ethyl ester for substrates 4j-k were prepared according to literature procedures¹⁰.

### Physical Characterization of Aldehyde Substrates

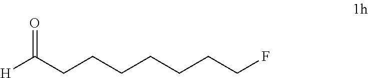

8-fluorooctanal (1h)

¹H NMR (600 MHZ, CDCl₃)δ 9.77 (t, J=1.9 Hz, 1H), 4.48 (t, J=6.1 Hz, 1H), 4.40 (t, J=6.1 Hz, 1H), 2.43 (td, J=7.3, 1.8 Hz, 2H), 1.77-1.62 (m, 4H), 1.47-1.32 (m, 6H). ¹³C NMR (151 MHz, CDCl₃) δ 202.75, 84.11 (d, J_{CF}=164.2 Hz), 43.85, 30.38, 30.25, 28.99 (d, J_{CF}=7.7 Hz), 25.01 (d, J_{CF}=5.5 Hz), 21.95. 19F NMR (376 MHz, CDCl₃) δ −220.88.

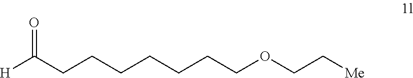 [SMILES: CCCOCCCCCCCC=O]

8-propoxyoctanal (1l)

¹H NMR (600 MHZ, CDCl₃) δ 9.76 (t, J=1.9 Hz, 1H), 3.39 (t, J=6.7 Hz, 2H), 3.36 (t, J=6.8 Hz, 2H), 2.42 (td, J=7.3, 1.8 Hz, 2H), 1.65-1.53 (m, 6H), 1.39-1.30 (m, 6H), 0.92 (t, J=7.4 Hz, 3H). ¹³C NMR (151 MHz, CDCl₃) δ 202.88, 72.59, 70.79, 43.90, 29.71, 29.21, 29.12, 26.02, 22.95, 22.03, 10.60.

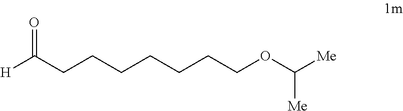 [SMILES: CC(C)OCCCCCCCC=O]

8-isopropoxyoctanal (1m)

¹H NMR (600 MHZ, CDCl₃) δ 9.76 (t, J=1.8 Hz, 1H), 3.58-3.50 (m, 1H), 3.39 (t, J=6.7 Hz, 2H), 2.42 (td, J=7.4, 1.8 Hz, 2H), 1.65-1.61 (m, 2H), 1.55 (p, J=6.7 Hz, 2H), 1.34 (qd, J=4.5, 2.8, 2.3 Hz, 6H), 1.15 (d, J=6.1 Hz, 6H). 13C NMR (151 MHz, CDCl₃) δ 202.91, 71.28, 68.11, 43.90, 30.10, 29.21, 29.11, 26.05, 22.16, 22.02.

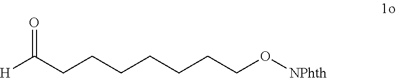

8-((1,3-dioxoisoindolin-2-yl)oxy)octanal (1o)

¹H NMR (600 MHZ, CDCl₃) δ 9.77 (t, J=1.9 Hz, 1H), 7.84 (dd, J=5.4, 3.1 Hz, 2H), 7.75 (dd, J=5.5, 3.0 Hz, 2H), 4.20 (t, J=6.7 Hz, 2H), 2.44 (td, J=7.4, 1.9 Hz, 2H), 1.79 (p, J=6.8 Hz, 2H), 1.66 (q, J=7.4 Hz, 2H), 1.54-1.48 (m, 2H), 1.39 (hd, J=8.9, 8.5, 2.8 Hz, 4H). ¹³C NMR (151 MHZ, CDCl₃) δ 202.85, 163.68, 134.46, 128.97, 123.49, 78.46, 43.84, 28.97, 28.06, 25.36, 21.96, 21.95.

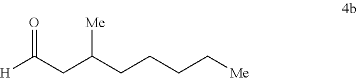

3-methyloctanal (4b)

¹H NMR (600 MHZ, CDCl₃) δ 9.76 (t, J=2.3 Hz, 1H), 2.39 (ddd, J=16.1, 5.8, 1.9 Hz, 1H), 2.22 (ddd, J=16.0, 7.9, 2.7 Hz, 1H), 2.04 (q, J=5.9, 4.0 Hz, 1H), 1.34-1.19 (m, 8H), 0.96 (dd, J=6.7, 1.1 Hz, 3H), 0.89 (td, J=7.1, 1.1 Hz, 3H). 13C NMR (151 MHz, CDCl₃) δ 203.22, 51.11, 36.88, 31.92, 28.19, 26.61, 22.62, 20.00, 14.06.

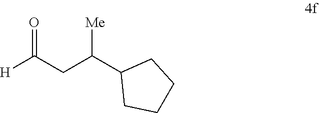

3-cyclopentylbutanal (4f)

¹H NMR (600 MHZ, CDCl₃) δ 9.94-9.64 (m, 1H), 2.50 (ddd, J=15.9, 4.3, 1.8 Hz, 1H), 2.22 (ddd, J=15.9, 9.0, 3.1 Hz, 1H), 1.92 (dddd, J=13.1, 8.7, 4.3, 1.9 Hz, 1H), 1.80-1.71 (m, 2H), 1.67-1.58 (m, 3H), 1.58-1.44 (m, 2H), 1.18-1.09 (m, 2H), 0.97 (d, J=6.7 Hz, 3H). ¹³C NMR (151 MHz, CDCl₃) δ 203.41, 50.25, 46.23, 33.53, 30.82, 30.29, 25.37, 18.83.

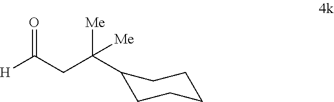 [SMILES: CC(C)(CC=O)C1CCCCC1]

3-cyclohexyl-3-methylbutanal (4k)

¹H NMR (600 MHZ, CDCl₃) δ 9.86 (t, J=3.3 Hz, 1H), 2.27 (d, J=3.3 Hz, 2H), 1.77 (t, J=12.7 Hz, 4H), 1.66 (d, J=11.9 Hz, 1H), 1.23-1.08 (m, 4H), 1.02 (s, 6H), 0.96 (qd, J=12.2, 10.8, 4.8 Hz, 2H). 13C NMR (151 MHz, CDCl₃) δ 203.91, 52.94, 47.03, 35.65, 26.77, 26.49, 26.07, 24.83.

### 4. Reaction Conditions Optimization

Part 1: Conditions Optimization for β-methylene C(sp³)—H Arylation

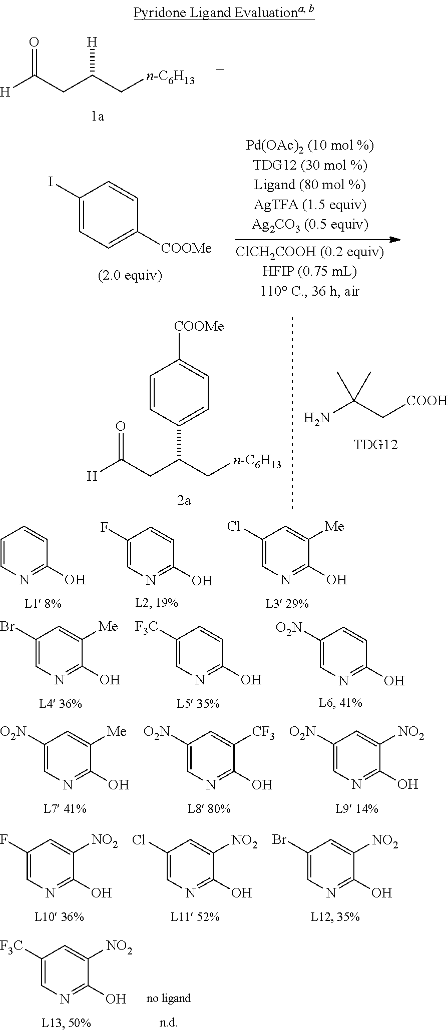

^{a}Conditions: 1a (0.1 mmol, 1.0 equiv), aryl iodide (2.0 equiv), Pd(OAc)₂ (10 mol %), TDG12 (30 mol %), Ligand (80 mol %), AgTFA (1.5 equiv), Ag₂CO₃ (0.5 equiv), ClCH₂COOH (0.2 equiv), HFIP (0.75 mL), 110° C., under air, 26 h. ^{b}Yield determined by ¹H NMR analysis of the crude product using CH₂Br₂ as internal standard.

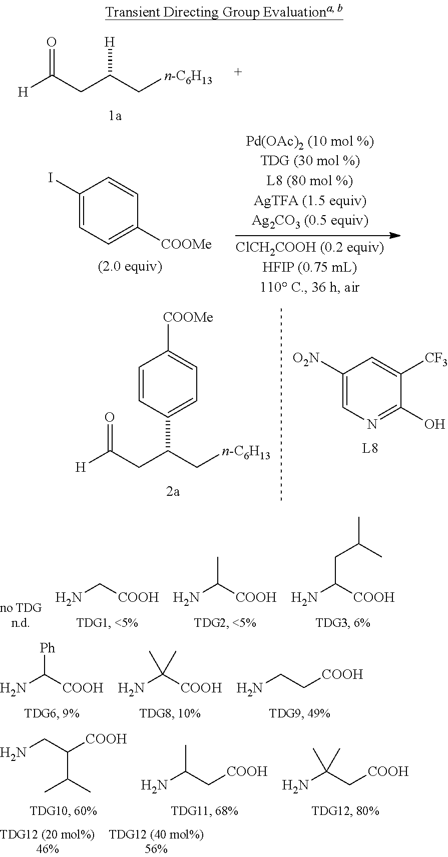

^{a}Conditions: 1a (0.1 mmol, 1.0 equiv), aryl iodide (2.0 equiv), Pd(OAc)₂ (10 mol %), TDG (30 mol %), L8 (80 mol %), AgTFA (1.5 equiv), Ag₂CO₃ (0.5 equiv), ClCH₂COOH (0.2 equiv), HFIP (0.75 mL), 110° C., under air, 26 h. ^{b}Yield determined by ¹H NMR analysis of the crude product using CH₂Br₂ as internal standard

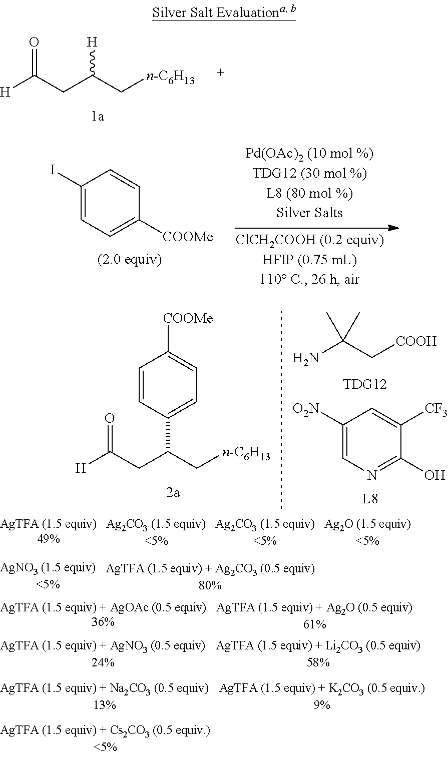

^{a}Conditions: 1a (0.1 mmol, 1.0 equiv), aryl iodide (2.0 equiv), Pd(OAc)₂ (10 mol %), TDG12 (30 mol %), L8 (80 mol %), silver salts, ClCH₂COOH (0.2 equiv), HFIP (0.75 mL), 110° C., under air, 26 h. ^{b}Yield determined by ¹H NMR analysis of the crude product using CH₂Br₂ as internal standard.

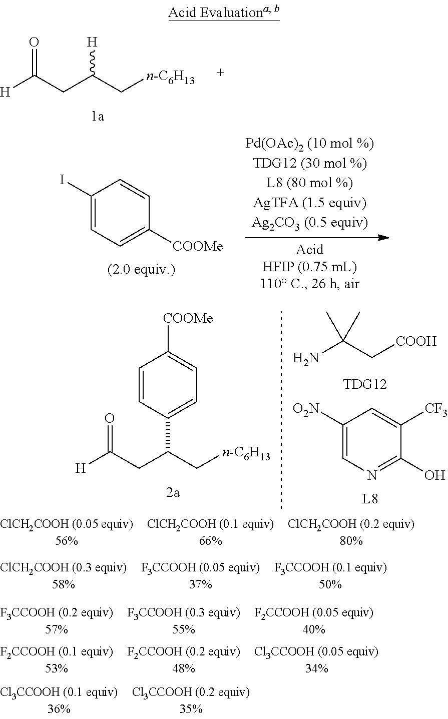

^{a}Conditions: 1a (0.1 mmol, 1.0 equiv), aryl iodide (2.0 equiv), Pd(OAc)₂ (10 mol %), TDG12 (30 mol %), L8 (80 mol %), AgTFA (1.5 equiv), Ag₂CO₃ (0.5 equiv), acid, HFIP (0.75 mL), 110° C., under air, 26 h. ^{b}Yield determined by ¹H NMR analysis of the crude product using CH₂Br₂ as internal standard.

Comparations between acetic acid and chloroacetic acid^{a,b}

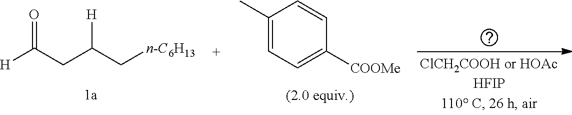

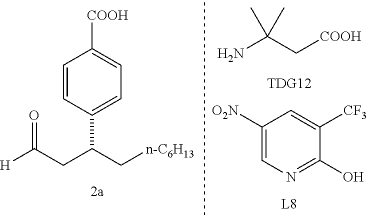

| | | ClCH₂COOH | HOAc | Solvent | Yield | Mass | |
|---|---|---|---|---|---|---|---|
| Entry | L8 (Y/N) | (equiv) | (equiv) | [0.133M] | (%) | (%) | Balance |
| 1^{c} | N | — | 19.8 | HFIP/HOAc (5:1, v/v) | 4 | 47 | |
| 2^{c} | Y | — | 19.8 | HFIP/HOAc (5:1, v/v) | 40 | 60 | |
| 3 | Y | — | — | HFIP | 46 | 96 | |
| 4 | Y | — | 0.2 | HFIP | 54 | 92 | |
| 5 | Y | — | 5.7 | HFIP/HOAc (20:1, v/v) | 62 | 72 | |
| 6 | Y | — | 19.8 | HFIP/HOAc (5:1, v/v) | 45 | 52 | |
| 7 | Y | 0.2 | — | HFIP | 80 | 96 | |

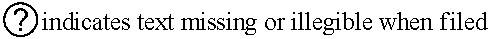

^{a}Conditions: 1a (0.1 mmol, 1.0 equiv), aryl iodide (2.0 equiv), Pd(OAc)₂ (10 mol %), TDG12 (30 mol %), L8
(80 mol %), AgTFA (1.5 equiv), Ag₂CO₃ (0.5 equiv), acid, solvent (0.75 mL), 110° C., under air, 26 h.
^{b}Yield and mass balance determined by ¹H NMR analysis of the crude product using CH₂Br₂ as internal standard.
^{c}Conditions: 1a (0.1 mmol, 1.0 equiv), aryl iodide (1.5 equiv), Pd(OAc)₂ (10 mol %), TDG12 (40 mol %), L8 (80 mol % or
None), AgTFA (1.5 equiv), HOAc/HFIP (5:1, v/v, 0.7 mL), 100° C., under N₂, 24 h.

### Part 2: Conditions Optimization for γ-C(sp3)-H Arylation

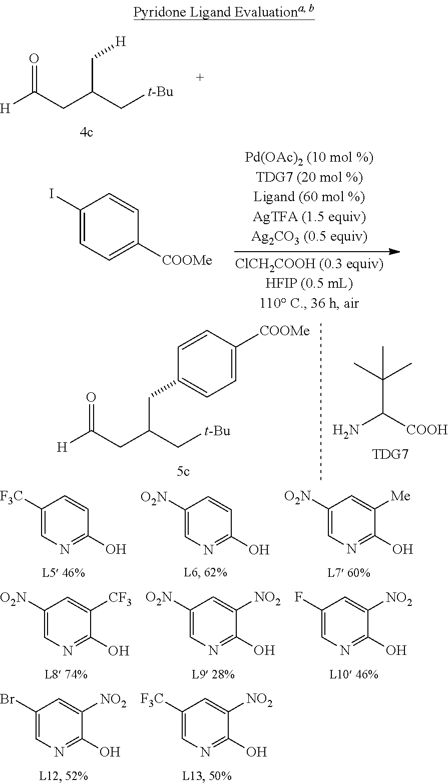

^{a}Conditions: 4c (0.1 mmol, 1.0 equiv), aryl iodide (2.0 equiv), Pd(OAc)₂ (10 mol %), TDG7 (20 mol %), Ligand (60 mol %), AgTFA (1.5 equiv), Ag₂CO₃ (0.5 equiv), ClCH₂COOH (0.3 equiv), HFIP (0.5 mL), 110° C., under air, 36 h. ^{b}Yield determined by ¹H NMR analysis of the crude product using CH₂Br₂ as internal standard.

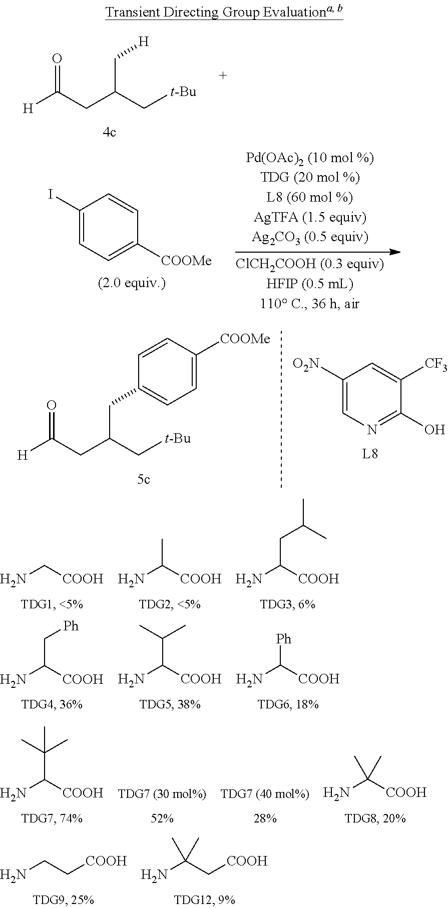

^{a}Conditions: 4c (0.1 mmol, 1.0 equiv), aryl iodide (2.0 equiv), Pd(OAc)₂ (10 mol%), TDG (20 mol %), L8 (60 mol %), AgTFA (1.5 equiv), Ag₂CO₃ (0.5 equiv), ClCH₂COOH (0.3 equiv), HFIP (0.5 mL), 110° C., under air, 36 h. ^{b}Yield determined by ¹H NMR analysis of the crude product using CH₂Br₂ as internal standard.

### 5. General Procedure for the B-Methylene and γ-C(sp³)—H Arylation.

### General Procedure A (for β-Methylene C(sp³)—H Arylation of Primary Aldehydes)

In air, to an oven-dried reaction tube (10 mL) equipped with a magnetic stir bar was added Pd(OAc)₂ (0.01 mmol, 10 mol %), transient directing groups (TDG12, 0.03 mmol, 30 mol %), ligand (L8, 0.08 mmol, 80 mol %), ArI (0.2 mmol, 2.0 equiv), AgTFA (0.15 mmol, 1.5 equiv), Ag₂CO₃ (0.05 mmol, 0.5 equiv), and solvent (HFIP, 0.75 mL and 0.02 mmol of ClCH₂COOH), followed by the aldehyde substrate (0.1 mmol, 1.0 equiv). The tube was sealed and stirred at room temperature for 20 min before heating to 110° C. for 26 h under vigorous stirring. Upon completion, the reaction mixture was cooled to room temperature and the dark brown suspension was diluted with 2 mL of ethyl acetate and was passed through a pad of Celite and washed with ethyl acetate (1.0 mL×3). The crude reaction mixture was purified on silica gel using hexanes/EtOAc as the eluent to afford the desired product.

### General Procedure B (for γ-C(sp³)—H Arylation of Primary Aldehydes)

In air, to an oven-dried reaction tube (10 mL) equipped with a magnetic stir bar was added Pd(OAc)₂ (0.01 mmol, 10 mol %), transient directing groups (TDG7, 0.02 mmol, 20 mol %), ligand (L8, 0.06 mmol, 60 mol %), ArI (0.2 mmol, 2.0 equiv), AgTFA (0.15 mmol, 1.5 equiv), Ag₂CO₃ (0.05 mmol, 0.5 equiv), and solvent (HFIP, 0.5 mL and 0.03 mmol of ClCH₂COOH), followed by the aldehyde substrate (0.1 mmol, 1.0 equiv). The tube was sealed and stirred at room temperature for 20 min before heating to 110° C. for 24 h under vigorous stirring. Upon completion, the reaction mixture was cooled to room temperature and the dark brown suspension was diluted with 2 mL of ethyl acetate and was passed through a pad of Celite and washed with ethyl acetate (1.0 mL×3). The crude reaction mixture was purified on silica gel using hexanes/EtOAc as the eluent to afford the desired product.

Physical Characterization of β-arylation Products

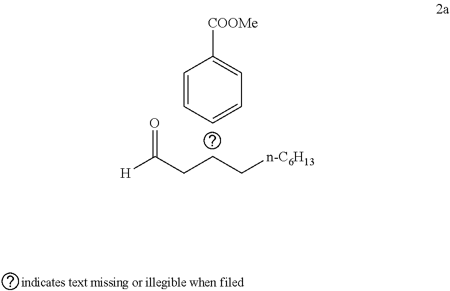

Methyl 4-(1-oxodecan-3-yl)benzoate (2a)

Following the General Procedure A, 2a was obtained as a colorless oil in 74% yield. ¹H NMR (600 MHz, CDCl₃) δ 9.67 (t, J=1.9 Hz, 1H), 7.99-7.96 (d, J=8.3 Hz, 2H), 7.26 (d, J=8.4 Hz, 2H), 3.90 (s, 3H), 3.27-3.20 (m, 1H), 2.74 (dd, J=7.3, 1.9 Hz, 2H), 1.70-1.58 (m, 2H), 1.27-1.07 (m, 10H), 0.85 (t, J=7.1 Hz, 3H). ¹³C NMR (151 MHz, CDCl₃) δ 201.23, 166.95, 149.52, 129.98, 128.57, 127.55, 52.05, 50.38, 39.96, 36.37, 31.75, 29.40, 29.08, 27.25, 22.60, 14.06. HRMS (ESI-TOF) Calcd for C₁₈H₂₅O₃⁻ [M−H]⁻: 289.1804, found: 289.1803.

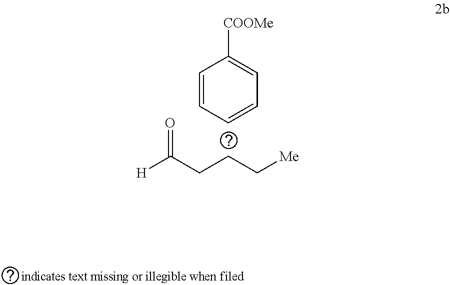

Methyl 4-(1-oxopentan-3-yl)benzoate (2b)

Following the General Procedure A, 2b was obtained as a colorless oil in 71% yield. ¹H NMR (600 MHz, CDCl₃) δ 9.68 (t, J=1.9 Hz, 1H), 7.98 (d, J=8.4 Hz, 2H), 7.26 (d, J=8.2 Hz, 2H), 3.90 (s, 3H), 3.16 (dtd, J=9.2, 7.2, 5.5 Hz, 1H), 2.79-2.70 (m, 2H), 1.78-1.61 (m, 2H), 0.80 (t, J=7.4 Hz, 3H). 13C NMR (151 MHz, CDCl3) 8 201.21, 166.95, 149.20, 129.96, 128.61, 127.62, 52.05, 49.98, 41.60, 29.29, 11.81. Spectroscopic data for this compound is consistent with that shown in the literature.¹¹

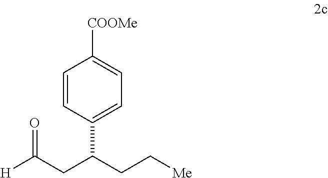 [SMILES: CCC[C@H](CC=O)c1ccc(C(=O)OC)cc1]

Methyl 4-(1-oxohexan-3-yl)benzoate (2c)

Following the General Procedure A, 2c was obtained as a colorless oil in 70% yield. ¹H NMR (600 MHZ, CDCl₃) δ 9.67 (s, 1H), 7.98 (d, J=8.4 Hz, 2H), 7.26 (d, J=7.9, 2H), 3.90 (s, 3H), 3.26 (p, J=7.2 Hz, 1H), 2.74 (dd, J=7.3, 1.9 Hz, 2H), 1.68-1.58 (m, 2H), 1.25-1.11 (m, 2H), 0.86 (t, J=7.3 Hz, 3H). ¹³C NMR (151 MHz, CDCl₃) δ 201.21, 166.93, 149.47, 129.98, 128.58, 127.55, 52.05, 50.34, 39.70, 38.54, 20.39, 13.88. HRMS (ESI-TOF) Calcd for C₁₄H₁₉O₃⁺ [M+H]⁺: 235.1334, found: 235.1335.

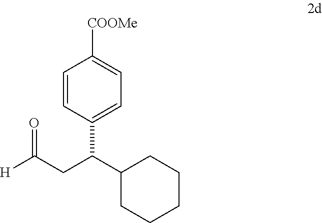 [SMILES: COC(=O)c1ccc([C@@H](CC=O)C2CCCCC2)cc1]

Methyl 4-(1-cyclohexyl-3-oxopropyl) benzoate (2d)

Following the General Procedure A, 2d was obtained as a colorless oil in 41% yield. ¹H NMR (600 MHz, CDCl₃) δ 9.62 (s, 1H), 7.96 (d, J=8.3 Hz, 2H), 7.22 (d, J=8.4 Hz, 2H), 3.90 (s, 3H), 3.06 (ddd, J=9.4, 7.8, 5.3 Hz, 1H), 2.92-2.71 (m, 2H), 1.84-1.71 (m, 2H), 1.67-1.58 (m, 2H), 1.54-1.47 (m, 1H), 1.46-1.39 (m, 1H), 1.22 (qt, J=12.8, 3.5 Hz, 1H), 1.16-1.03 (m, 2H), 0.94 (qd, J=12.5, 3.5 Hz, 1H), 0.81 (qd, J=12.3, 3.6 Hz, 1H). 13C NMR (151 MHZ, CDCl₃) δ 201.73, 166.96, 148.45, 129.72, 128.50, 128.33, 52.04, 47.00, 45.98, 42.97, 31.04, 30.70, 26.35, 26.24, 26.23. HRMS (ESI-TOF) Calcd for C₁₇H₂₃O₃⁺ [M+H]⁺: 275.1647, found: 275.1649

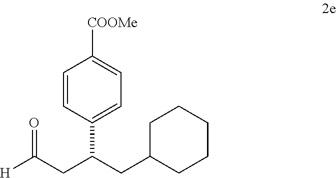 [SMILES: COC(=O)c1ccc([C@@H](CC=O)CC2CCCCC2)cc1]

Methyl 4-(1-cyclohexyl-4-oxobutan-2-yl)benzoate (2e)

Following the General Procedure A, 2e was obtained as a colorless oil in 69% yield. ¹H NMR (600 MHZ, CDCl₃) δ 9.65 (t, J=1.9 Hz, 1H), 7.98 (d, J=8.3 Hz, 2H), 7.27 (d, J=7.1 Hz, 2H), 3.90 (s, 3H), 3.39 (dtd, J=9.9, 7.2, 5.5 Hz, 1H), 2.70 (dt, J=7.0, 1.9 Hz, 2H), 1.83-1.76 (m, 1H), 1.69-1.60 (m, 2H), 1.59-1.44 (m, 4H), 1.09 (dq, J=12.7, 6.4, 3.7 Hz, 3H), 1.00 (tddd, J=11.7, 8.3, 4.9, 2.4 Hz, 1H), 0.94-0.84 (m, 2H). ¹³C NMR (151 MHZ, CDCl₃) δ 201.24, 166.96, 149.64, 130.02, 128.55, 127.54, 52.05, 50.86, 44.11, 36.94, 34.71, 33.91, 32.57, 26.47, 26.10, 26.01. HRMS (ESI-TOF) Calcd for C₁₈H₂₅O₃⁺ [M+H]⁺: 289.1804, found: 289.1810.

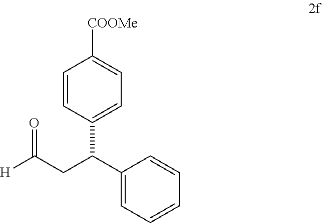 [SMILES: COC(=O)c1ccc([C@@H](CC=O)c2ccccc2)cc1]

Methyl 4-(3-oxo-1-phenylpropyl)benzoate (2f)

Following the General Procedure A, 2f was obtained as a colorless oil in 50% yield. ¹H NMR (600 MHz, CDCl₃) δ 9.75 (d, J=1.4 Hz, 1H), 7.96 (d, J=7.8 Hz, 2H), 7.30 (t, J=7.3 Hz, 4H), 7.21 (d, J=8.1 Hz, 3H), 4.68 (t, J=7.7 Hz, 1H), 3.89 (s, 3H), 3.21 (d, J=7.6 Hz, 2H). ¹³C NMR (151 MHz, CDCl₃) 200.27, 166.79, 148.48, 142.39, 130.07, 128.89, 128.65, 127.78, 127.72, 127.00, 52.10, 49.15, 44.79. HRMS (ESI-TOF) Calcd for C₁₇H₁₇O₃⁺ [M+H]⁺: 269.1178, found: 269.1180.

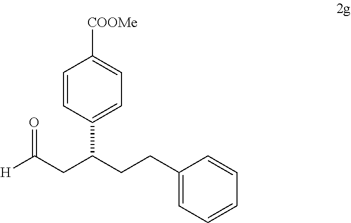 [SMILES: COC(=O)c1ccc([C@@H](CC=O)CCc2ccccc2)cc1]

Methyl 4-(1-oxo-5-phenylpentan-3-yl)benzoate (2g)

Following the General Procedure A, 2g was obtained as a colorless oil in 56% yield. ¹H NMR (600 MHZ, CDCl₃) δ 9.64 (t, J=1.8 Hz, 1H), 8.01 (d, J=8.2 Hz, 2H), 7.29 (d, J=8.3 Hz, 2H), 7.27-7.24 (m, 2H), 7.20-7.16 (m, 1H), 7.08 (d, J=6.7 Hz, 2H), 3.92 (s, 3H), 3.28 (dtd, J=9.8, 7.2, 5.0 Hz, 1H), 2.77 (dd, J=7.2, 1.8 Hz, 2H), 2.46 (t, J=8.0 Hz, 2H), 2.07-1.92 (m, 2H). ¹³C NMR (151 MHz, CDCl₃) δ 200.83, 166.89, 148.86, 141.31, 130.12, 128.81, 128.44, 128.30, 127.69, 126.02, 52.09, 50.43, 39.41, 37.79, 33.35. HRMS (ESI-TOF) Calcd for C₁₉H₂₁O₃⁺ [M+H]⁺: 297.1491, found: 297.1489.

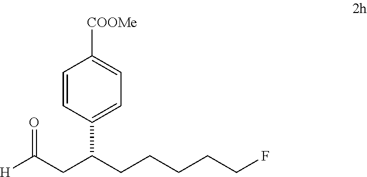 [SMILES: COC(=O)c1ccc([C@@H](CC=O)CCCCCF)cc1]

Methyl 4-(8-fluoro-1-oxooctan-3-yl)benzoate (2h)

Following the General Procedure A, 2h was obtained as a colorless oil in 81% yield. ¹H NMR (600 MHZ, CDCl₃) δ 9.67 (t, J=1.8 Hz, 1H), 7.98 (d, J=8.3 Hz, 2H), 7.26 (d, J=8.4 Hz, 2H), 4.41 (t, J=6.1 Hz, 1H), 4.33 (t, J=6.1 Hz, 1H), 3.91 (s, 3H), 3.30-3.21 (m, 1H), 2.76 (dd, J=7.2, 1.8 Hz, 2H), 1.74-1.58 (m, 4H), 1.42-1.29 (m, 2H), 1.24 (ddt, J=11.7, 9.5, 5.7 Hz, 1H), 1.18-1.10 (m, 1H). ¹³C NMR (151 MHz, CDCl₃) δ 201.01, 166.90, 149.24, 130.03, 128.68, 127.53, 83.93 (d, J_{CF}=164.2 Hz), 52.08 (d, J_{CF}=2.7 Hz), 50.38, 39.79, 36.16, 30.17 (d, J_{CF}=19.3 Hz), 26.87, 25.05 (d, J_{CF}=5.4 Hz). ¹⁹F NMR (376 MHZ, CDCl₃) δ −221.05. HRMS (ESI-TOF) Calcd for C₁₆H₂₂FO₃⁺ [M+H]⁺: 281.1553, found: 281.1553.

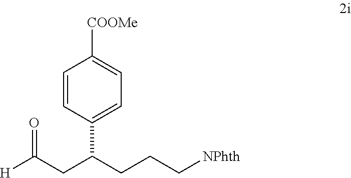

Methyl 4-(6-(1,3-dioxoisoindolin-2-yl)-1-oxohexan-3-yl)benzoate (2i)

Following the General Procedure A, reaction time 32 h, 2i was obtained as a pale-yellow solid in 62% yield. ¹H NMR (600 MHZ, CDCl₃) δ 9.65 (s, 1H), 7.96 (d, J=7.9 Hz, 2H), 7.82 (dd, J=5.5, 3.1 Hz, 2H), 7.70 (dd, J=5.5, 3.0 Hz, 2H), 7.26 (d, J=8.0 Hz, 2H), 3.89 (s, 3H), 3.63 (t, J=7.1 Hz, 2H), 3.35-3.26 (m, 1H), 2.76 (d, J=7.1 Hz, 2H), 1.78-1.66 (m, 2H), 1.59 (qd, J=11.5, 10.0, 6.6 Hz, 1H), 1.49 (hept, J=8.4, 7.7 Hz, 1H). 13C NMR (151 MHZ, CDCl₃) δ 200.69, 168.34, 166.81, 148.60, 133.96, 132.01, 130.11, 128.80, 127.54, 123.23, 52.05, 50.21, 39.38, 37.53, 33.31, 26.34. HRMS (ESI-TOF) Calcd for C₂₂H₂₂NO₅⁺ [M+H]⁺: 380.1498, found: 380.1495.

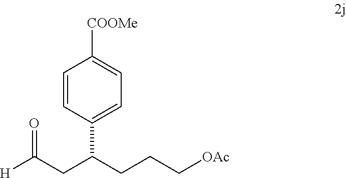 [SMILES: COC(=O)c1ccc([C@@H](CC=O)CCCOC(C)=O)cc1]

Methyl 4-(6-acetoxy-1-oxohexan-3-yl)benzoate (2j)

Following the General Procedure A, reaction time 72 h, 2j was obtained as a colorless oil in 70% yield. ¹H NMR (600 MHz, CDCl₃) δ 9.68 (t, J=1.7 Hz, 1H), 7.99 (d, J=8.5 Hz, 2H), 7.28 (s, 2H), 4.00 (t, J=6.5 Hz, 2H), 3.91 (s, 3H), 3.28 (dtd, J=9.7, 7.1, 5.2 Hz, 1H), 2.78 (dd, J=6.9, 1.4 Hz, 2H), 2.02 (s, 3H), 1.82-1.63 (m, 2H), 1.57-1.38 (m, 2H). ¹³C NMR (151 MHz, CDCl₃) δ 200.65, 171.08, 166.84, 148.67, 130.12, 127.54, 127.53, 63.95, 52.10, 50.36, 39.48, 32.51, 26.44, 20.94. HRMS (ESI-TOF) Calcd for C₁₆H₂₁O₅⁺ [M+H]⁺: 293.1384, found: 293.1384.

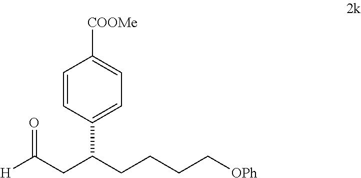 [SMILES: COC(=O)c1ccc([C@@H](CC=O)CCCCOc2ccccc2)cc1]

Methyl 4-(1-oxo-7-phenoxyheptan-3-yl)benzoate (2k)

Following the General Procedure A, 2k was obtained as a colorless oil in 61% yield. ¹H NMR (600 MHZ, CDCl₃) δ 9.67 (s, 1H), 7.98 (d, J=8.2 Hz, 2H), 7.27-7.23 (m, 4H), 6.92 (t, J=7.3 Hz, 1H), 6.83 (d, J=7.6 Hz, 2H), 3.92-3.85 (m, 5H), 3.32-3.25 (m, 1H), 2.77 (dd, J=7.2, 1.8 Hz, 2H), 1.81-1.67 (m, 4H), 1.39-1.22 (m, 2H). ¹³C NMR (151 MHz, CDCl₃) δ 200.97, 166.90, 158.90, 149.14, 130.05, 129.41, 128.70, 127.56, 120.58, 114.44, 67.36, 52.07, 50.35, 39.83, 35.99, 29.03, 23.86. HRMS (ESI-TOF) Calcd for C₂₁H₂₃O₄ [M−H]⁻: 339.1596, found: 339.1603.

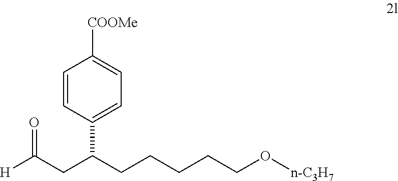

Methyl 4-(1-oxo-8-propoxyoctan-3-yl)benzoate (2l)

Following the General Procedure A, 21 was obtained as a colorless oil in 69% yield. ¹H NMR (600 MHZ, CDCl₃) δ 9.66 (t, J=1.8 Hz, 1H), 7.98 (d, J=8.3 Hz, 2H), 7.26 (d, J=8.4 Hz, 2H), 3.90 (s, 3H), 3.35-3.30 (m, 4H), 3.27-3.21 (m, 1H), 2.74 (dd, J=7.2, 1.9 Hz, 2H), 1.70- 1.62 (m, 2H), 1.56 (dt, J=14.3, 7.1 Hz, 2H), 1.48 (tdd, J=10.4, 5.5, 3.0 Hz, 2H), 1.37-1.25 (m, 2H), 1.25-1.18 (m, 1H), 1.12 (tdd, J=13.1, 10.2, 5.3 Hz, 1H), 0.89 (t, J=7.4 Hz, 3H). 13C NMR (151 MHZ, CDCl₃) δ 201.13, 166.92, 149.41, 129.99, 128.60, 127.55, 72.55, 70.60, 52.05, 50.37, 39.88, 36.29, 29.53, 27.07, 26.05, 22.94, 10.58. HRMS (ESI-TOF) Calcd for C₁₉H₂₇O₄ [M−H]⁻: 319.1909, found: 319.1913.

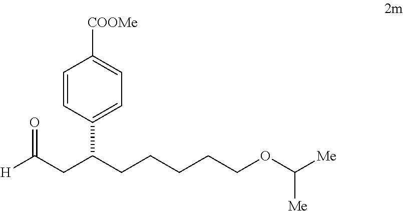 [SMILES: COC(=O)c1ccc([C@@H](CC=O)CCCCCOC(C)C)cc1]

Methyl 4-(8-isopropoxy-1-oxooctan-3-yl)benzoate (2m)

Following the General Procedure A, 2m was obtained as a colorless oil in 65% yield. 1H NMR (600 MHZ, CDCl₃) δ 9.66 (s, 1H), 7.98 (d, J=8.0 Hz, 2H), 7.26 (d, J=8.1 Hz, 2H), 3.90 (s, 3H), 3.49 (hept, J=6.1 Hz, 1H), 3.32 (t, J=6.6 Hz, 2H), 3.28-3.21 (m, 1H), 2.74 (dd, J=7.2, 1.9 Hz, 2H), 1.66 (ttd, J=18.2, 9.1, 8.6, 5.1 Hz, 2H), 1.47 (ddt, J=9.6, 6.6, 4.8 Hz, 2H), 1.39-1.13 (m, 4H), 1.11 (d, J=6.2 Hz, 6H). 13C NMR (151 MHz, CDCl₃) δ 201.16, 166.92, 149.41, 129.99, 128.59, 127.54, 71.26, 67.92, 52.05, 50.35, 39.89, 36.28, 29.91, 27.07, 26.09, 22.13. HRMS (ESI-TOF) Calcd for C₁₉H₂₇O₄ [M−H]⁻: 319.1909, found: 319.1910.

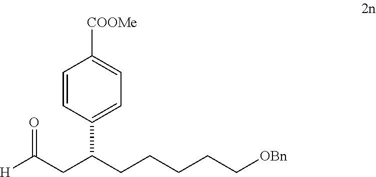 [SMILES: COC(=O)c1ccc([C@@H](CC=O)CCCCCOCc2ccccc2)cc1]

Methyl 4-(8-(benzyloxy)-1-oxooctan-3-yl) benzoate (2n)

Following the General Procedure A, 2n was obtained as a colorless oil in 72% yield. ¹H NMR (600 MHZ, CDCl₃) ¹H NMR (600 MHZ, CDCl₃) δ 9.65 (s, 1H), 7.97 (d, J=7.9 Hz, 2H), 7.32 (dt, J=13.2, 7.3 Hz, 4H), 7.25 (d, J=8.1 Hz, 3H), 4.46 (s, 2H), 3.90 (s, 3H), 3.40 (t, J=6.5 Hz, 2H), 3.27-3.17 (m, 1H), 2.73 (d, J=5.3 Hz, 2H), 1.67-1.48 (m, 4H), 1.42-1.28 (m, 2H), 1.23-1.06 (m, 2H). ¹³C NMR (151 MHz, CDCl₃) δ 201.12, 166.91, 149.38, 138.60,129.98, 128.59, 128.34, 127.60, 127.53, 127.49, 72.85, 70.18, 52.04, 50.35, 39.86, 36.25, 29.50, 27.04, 26.03. HRMS (ESI-TOF) Calcd for C₂₃H₂₉O₄⁺ [M+H]⁺: 369.2066, found: 369.2058.

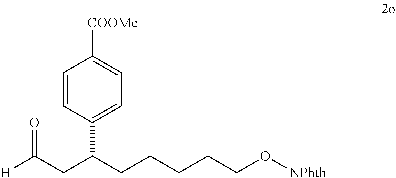

Methyl 4-(8- ((1,3-dioxoisoindolin-2-yl) oxy)-1-oxooctan-3-yl)benzoate (20)

Following the General Procedure A, 20 was obtained as a pale-yellow solid in 71% yield. ¹H NMR (600 MHZ, CDCl₃) δ 9.67 (t, J=1.8 Hz, 1H), 7.98 (d, J=8.2 Hz, 2H), 7.82 (dt, J=7.0, 3.6 Hz, 2H), 7.76-7.73 (m, 2H), 7.28 (d, J=8.3 Hz, 2H), 4.14 (t, J=6.6 Hz, 2H), 3.90 (s, 3H), 3.27 (ddt, J=12.7, 9.3, 6.1 Hz, 1H), 2.76 (dt, J=7.7, 1.4 Hz, 2H), 1.77-1.65 (m, 4H), 1.56-1.41 (m, 2H), 1.31-1.15 (m, 2H). ¹³C NMR (151 MHz, CDCl₃) δ 201.11, 166.91, 163.64, 149.29, 134.46, 130.02, 128.95, 128.62, 127.56, 123.49, 52.04, 50.32, 39.81, 36.14, 27.91, 26.86, 25.42. HRMS (ESI-TOF) Calcd for C₂₄H₂₆NO₆⁺ [M+H]⁺: 424.1760, found: 424.1757.

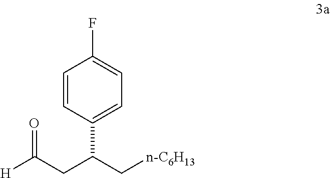

3-(4-fluorophenyl)decanal (3a)

Following the General Procedure A, reaction time 28 h, 3a was obtained as a colorless oil in 80% yield. ¹H NMR (600 MHZ, CDCl₃) δ 9.66 (t, J=2.0 Hz, 1H), 7.16-7.12 (m, 2H), 7.03-6.95 (m, 2H), 3.20-3.10 (m, 1H), 2.74-2.64 (m, 2H), 1.67-1.55 (m, 2H), 1.28-1.09 (m, 10H), 0.85 (t, J=7.2 Hz, 3H). ¹³C NMR (151 MHZ, CDCl₃) ¹³C NMR (151 MHz, CDCl₃) δ 201.71, 161.49 (d, J_{CF}=244.3 Hz), 139.63 (d, J_{CF}=3.3 Hz), 128.82 (d, J_{CF}=7.7 Hz), 115.40 (d, J_{CF}=20.9 Hz), 50.76, 39.32, 36.70, 31.77, 29.40, 29.12, 27.24, 22.61, 14.07. ¹⁹F NMR (376 MHz, CDCl₃) δ −119.23. HRMS (ESI-TOF) Calcd for C₁₆H₂₃FNaO⁺ [M+Na]⁺: 273.1625, found: 273.1610.

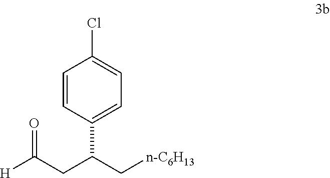

3-(4-chlorophenyl)decanal (3b)

Following the General Procedure A, reaction time 28 h, 3b was obtained as a colorless oil in 69% yield. ¹H NMR (600 MHZ, CDCl₃) δ 9.66 (t, J=2.0 Hz, 1H), 7.27 (d, J=8.3 Hz, 2H), 7.12 (d, J=8.4 Hz, 2H), 3.15 (dq, J=9.4, 6.9 Hz, 1H), 2.74-2.65 (m, 2H), 1.67-1.56 (m, 2H), 1.26-1.07 (m, 10H), 0.85 (t, J=7.2 Hz, 3H). ¹³C NMR (151 MHz, CDCl₃) δ 201.48, 142.50, 132.18, 128.82, 128.75, 50.58, 39.41, 36.51, 31.77, 29.40, 29.11, 27.23, 22.61, 14.06. HRMS (ESI-TOF) Calcd for C₁₆H₂₃ClNaO⁺ [M+Na]⁺: 289.1329, found: 289.1329.

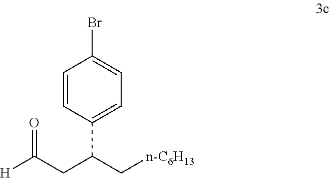

3-(4-bromophenyl)decanal (3c)

Following the General Procedure A, reaction time 28 h, 3f was obtained as a colorless oil in 70% yield. ¹H NMR (600 MHZ, CDCl₃) δ 9.66 (t, J=1.9 Hz, 1H), 7.42 (d, J=8.5 Hz, 2H), 7.06 (d, J=8.2 Hz, 2H), 3.17-3.09 (m, 1H), 2.73-2.66 (m, 2H), 1.68-1.56 (m, 2H), 1.26-1.08 (m, 10H), 0.85 (t, J=7.2 Hz, 3H). ¹³C NMR (151 MHz, CDCl₃) ¹³C NMR (151 MHZ, CDCl₃) δ 201.42, 143.04, 131.70, 129.22, 120.22, 50.52, 39.46, 36.45, 31.77, 29.40, 29.10, 27.23, 22.60, 14.06. HRMS (ESI-TOF) Calcd for C₁₆H₂₃BrNaO⁺ [M+Na]⁺: 333.0824, found: 333.0808.

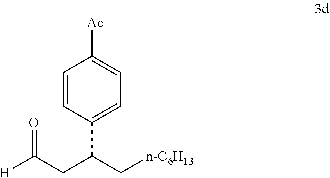

3-(4-acetylphenyl)decanal (3d)

Following the General Procedure A, reaction time 28 h, 3g was obtained as a colorless oil in 69% yield. ¹H NMR (600 MHz, CDCl₃) δ 9.67 (t, J=1.8 Hz, 1H), 7.91 (d, J=8.4 Hz, 2H), 7.29 (d, J=8.2 Hz, 2H), 3.25 (dtd, J=9.3, 7.2, 5.6 Hz, 1H), 2.79-2.72 (m, 2H), 2.59 (s, 3H), 1.70-1.59 (m, 2H), 1.25-1.04 (m, 10H), 0.85 (t, J=7.1 Hz, 3H). ¹³C NMR (151 MHZ, CDCl₃) δ 201.15, 197.71, 149.80, 135.71, 128.79, 127.74, 50.35, 39.93, 36.35, 31.76, 29.40, 29.09, 27.26, 26.58, 22.60, 14.06. HRMS (ESI-TOF) Calcd for C₁₈H₂₇O₂⁺ [M+H]⁺: 275.2011, found: 275.2018.

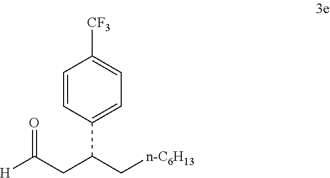

3-(4-(trifluoromethyl)phenyl)decanal (3e)

Following the General Procedure A, reaction time 24 h, 3h was obtained as a colorless oil in 71% yield. ¹H NMR (600 MHZ, CDCl₃) δ 9.68 (d, J=1.8 Hz, 1H), 7.56 (d, J=7.9 Hz, 2H), 7.30 (d, J=7.9 Hz, 2H), 3.29-3.21 (m, 1H), 2.75 (dq, J=7.6, 1.6 Hz, 2H), 1.72-1.58 (m, 2H), 1.28-1.05 (m, 10H), 0.85 (t, J=7.2 Hz, 3H). ¹³C NMR (151 MHz, CDCl₃) δ 201.04,148.25, 128.88 (q, J_{CF}=32.5 Hz), 127.85, 125.58 (q, J_{CF}=3.9 Hz), 125.57 (q, J_{CF}=270.2Hz), 50.42, 39.74, 36.37, 31.76, 29.39, 29.08, 27.24, 22.60, 14.06. 19F NMR (376 MHZ, CDCl₃) δ −65.07. HRMS (ESI-TOF) Calcd for C₁₇H₂₃F₃NaO₃⁺ [M+Na]⁺: 323.1593, found: 323.1599.

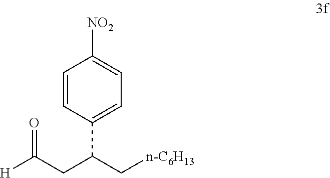

3-(4-nitrophenyl)decanal (3f)

Following the General Procedure A, reaction time 28 h, 3f was obtained as a colorless oil in 83% yield. ¹H NMR (600 MHZ, CDCl₃) δ 9.69 (t, J=1.6 Hz, 1H), 8.17 (d, J=8.7 Hz, 2H), 7.36 (d, J=8.7 Hz, 2H), 3.35-3.28 (m, 1H), 2.86-2.73 (m, 2H), 1.72-1.58 (m, 2H), 1.27-1.05 (m, 10H), 0.85 (t, J=7.2 Hz, 3H). ¹³C NMR (151 MHz, CDCl₃) δ 200.33, 152.02, 146.73, 128.38, 123.91, 50.28, 39.65, 36.26, 31.72, 29.35, 29.05, 27.24, 22.58, 14.05. HRMS (ESI-TOF) Calcd for C₁₆H₂₄NO₃⁺ [M+H]⁺: 278.1751, found: 278.1751.

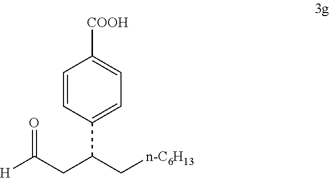

4-(1-oxodecan-3-yl)benzoic acid (3g)

Following the General Procedure A, reaction time 36 h, 3g was obtained as a white solid in 57% yield. ¹H NMR (600 MHZ, CDCl₃) ¹H NMR (600 MHz, CDCl₃) δ 9.68 (t, J=1.8 Hz, 1H), 8.05 (d, J=8.3 Hz, 2H), 7.30 (d, J=8.3 Hz, 2H), 3.26 (p, J=6.9 Hz, 1H), 2.76 (dd, J=7.3, 1.8 Hz, 2H), 1.65 (ttd, J=17.7, 8.8, 8.4, 4.8 Hz, 2H), 1.26-1.05 (m, 10H), 0.85 (t, J=7.1 Hz, 3H). ¹³C NMR (151 MHz, CDCl₃) δ 201.12, 170.92, 150.56, 130.63, 127.70, 127.58, 50.35, 40.00, 36.35, 31.75, 29.40, 29.08, 27.26, 22.60, 14.06. HRMS (ESI-TOF) Calcd for C₁₇H₂₄NaO₃⁺ [M+Na]⁺: 299.1617, found: 299.1622.

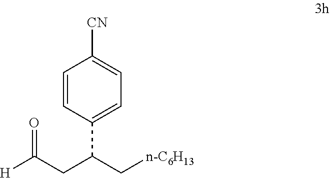

4-(1-oxodecan-3-yl)benzonitrile (3h)

Following the General Procedure A, reaction time 28 h, 3h was obtained as a colorless oil in 45% yield. ¹H NMR (600 MHz, CDCl₃) ¹H NMR (600 MHZ, CDCl₃) δ 9.67 (t, J=1.6 Hz, 1H), 7.60 (d, J=8.3 Hz, 2H), 7.30 (d, J=8.3 Hz, 2H), 3.25 (tt, J=8.0, 5.9 Hz, 1H), 2.81-2.69 (m, 2H), 1.70-1.58 (m, 2H), 1.26-1.03 (m, 10H), 0.85 (t, J=7.1 Hz, 3H). ¹³C NMR (151 MHz, CDCl₃) ¹³C NMR (151 MHz, CDCl₃) δ 200.52, 149.83, 132.47, 128.36, 118.84,110.49, 50.23, 39.88, 36.20, 31.73, 29.35, 29.05, 27.22, 22.58, 14.05. HRMS (ESI-TOF) Calcd for C₁₇H₂₄NO⁺ [M+H]⁺: 258.1853, found: 258.1847.

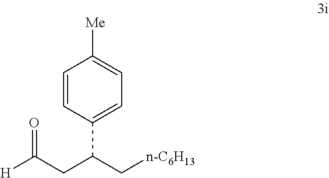

3-(p-tolyl) decanal (3i)

Following the General Procedure A, reaction time 28 h, 3i was obtained as a colorless oil in 60% yield. ¹H NMR (600 MHz, CDCl₃) δ 9.65 (t, J=2.1 Hz, 1H), 7.11 (d, J=7.7 Hz, 2H), 7.07 (d, J=6.4 Hz, 2H), 3.12 (p, J=7.4 Hz, 1H), 2.68 (d, J=7.4 Hz, 2H), 2.32 (s, 3H), 1.60 (dq, J=13.8, 7.9, 6.6 Hz, 2H), 1.29-1.09 (m, 10H), 0.88-0.83 (m, 3H). ¹³C NMR (151 MHz, CDCl₃) δ 202.35, 140.89, 136.03, 129.29, 127.31, 50.69, 39.75, 36.68, 31.81, 29.48, 29.13, 27.31, 22.62, 21.01, 14.08. HRMS (ESI-TOF) Calcd for C₁₇H₂₆NaO⁺ [M+Na]⁺: 269.1876, found: 269.1868.

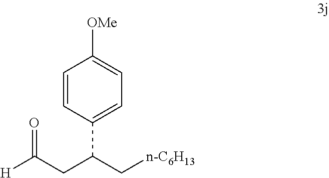

3-(4-methoxyphenyl)decanal (3j)

Following the General Procedure A, reaction time 28 h, 3j was obtained as a colorless oil in 52% yield. ¹H NMR (600 MHz, CDCl₃) δ 9.65 (t, J=2.2 Hz, 1H), 7.09 (d, J=8.4 Hz, 2H), 6.84 (d, J=8.7 Hz, 2H), 3.79 (s, 3H), 3.11 (dtd, J=9.2, 7.3, 5.6 Hz, 1H), 2.67 (dd, J=7.4, 2.2 Hz, 2H), 1.65-1.56 (m, 2H), 1.29-1.08 (m, 10H), 0.85 (t, J=7.1 Hz, 3H). ¹³C NMR (151 MHz, CDCl₃) δ 202.36, 158.16, 135.96, 128.35, 113.98, 55.23, 50.80, 39.36, 36.80, 31.81, 29.46, 29.15, 27.29, 22.62, 14.08. HRMS (ESI-TOF) Calcd for C₁₇H₂₆NaO₂⁺ [M+Na]⁺: 285.1825, found: 285.1824.

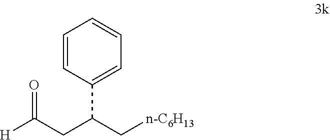

3-phenyldecanal (3k)

Following the General Procedure A, reaction time 28 h, 3k was obtained as a colorless oil in 55% yield. ¹H NMR (600 MHZ, CDCl₃) δ 9.66 (d, J=3.4 Hz, 1H), 7.30 (t, J=7.2 Hz, 2H), 7.20 (dd, J=17.9, 7.8 Hz, 3H), 3.16 (p, J=7.3 Hz, 1H), 2.76-2.66 (m, 2H), 1.63 (dtd, J=13.2, 7.8, 6.9, 3.4 Hz, 2H), 1.30-1.09 (m, 10H), 0.85 (t, J=7.2 Hz, 3H). ¹³C NMR (151 MHZ, CDCl₃) δ 202.15, 143.97, 128.61, 127.46, 126.54, 50.62, 40.12, 36.62, 31.79, 29.46, 29.12, 27.30, 22.61, 14.07. HRMS (ESI-TOF) Calcd for C₁₆H₂₄NaO⁺ [M+Na]⁺: 255.1719, found: 255.1718.

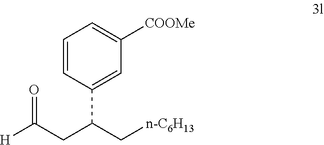

Methyl 3-(1-oxodecan-3-yl)benzoate (31)

Following the General Procedure A, reaction time 28 h, 31 was obtained as a colorless oil in 72% yield. ¹H NMR (600 MHZ, CDCl₃) δ 9.67 (t, J=1.9 Hz, 1H), 7.91-7.84 (m, 2H), 7.41- 7.35 (m, 2H), 3.92 (s, 3H), 3.23 (dtd, J=9.1, 7.2, 5.7 Hz, 1H), 2.75 (dd, J=7.2, 1.9 Hz, 2H), 1.72-1.60 (m, 2H), 1.27-1.06 (m, 10H), 0.85 (t, J=7.2 Hz, 3H). ¹³C NMR (151 MHZ, CDCl₃) δ 201.43, 167.09, 144.50, 132.33, 130.51, 128.68, 128.42, 127.87, 52.14, 50.51, 39.84, 36.49, 31.76, 29.40, 29.09, 27.27, 22.60, 14.06. HRMS (ESI-TOF) Calcd for C₁₈H₂₇O₃⁺ [M+H]⁺: 291.1960, found: 291.1967.

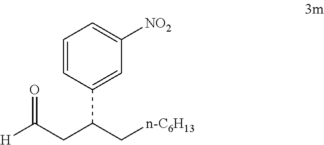

3-(3-nitrophenyl)decanal (3m)

Following the General Procedure A, reaction time 28 h, 3m was obtained as a colorless oil in 78% yield. ¹H NMR (600 MHZ, CDCl₃) δ 9.70 (t, J=1.5 Hz, 1H), 8.11-8.04 (m, 2H), 7.54 (dt, J=7.7, 1.4 Hz, 1H), 7.48 (t, J=7.8 Hz, 1H), 3.36-3.28 (m, 1H), 2.86-2.75 (m, 2H), 1.73-1.60 (m, 2H), 1.28-1.06 (m, 10H), 0.85 (t, J=7.2 Hz, 3H). ¹³C NMR (151 MHZ, CDCl₃) δ 200.43, 148.53, 146.40, 134.10, 129.50, 122.17, 121.74, 50.37, 39.48, 36.32, 31.73, 29.33, 29.06, 27.24, 22.58, 14.05. HRMS (ESI-TOF) Calcd for C₁₆H₂₃NNaO₃⁺ [M+Na]⁺: 300.1570, found: 300.1573.

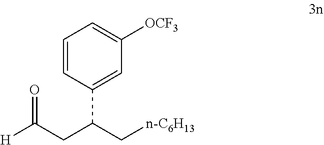

3-(3-(trifluoromethoxy)phenyl)decanal (3n)

Following the General Procedure A, reaction time 28 h, 3n was obtained as a colorless oil in 63% yield. ¹H NMR (600 MHZ, CDCl₃) ¹H NMR (600 MHZ, CDCl₃) δ 9.68 (t, J=1.8 Hz, 1H), 7.32 (t, J=7.9 Hz, 1H), 7.12 (dd, J=7.7, 1.4 Hz, 1H), 7.07 (ddt, J=8.1, 2.2, 1.1 Hz, 1H), 7.03 (t, J=1.3 Hz, 1H), 3.20 (dtd, J=9.3, 7.1, 5.5 Hz, 1H), 2.72 (ddd, J=7.5, 1.8, 1.0 Hz, 2H), 1.68-1.56 (m, 2H), 1.27-1.08 (m, 10H), 0.85 (t, J=7.2 Hz, 3H). ¹³C NMR (151 MHZ, CDCl₃) δ 201.15, 149.49 (q, J_{CF}=1.7 Hz), 146.55, 129.90, 125.93, 120.46 (q, J_{CF}=257.0 Hz), 119.98, 118.89, 50.44, 39.65, 36.36, 31.74, 29.35, 29.06, 27.17, 22.59, 14.05. 19F NMR (376 MHz, CDCl₃) δ −60.41. HRMS (ESI-TOF) Calcd for C₁₇H₂₃NaF₃O₂⁺ [M+Na]⁺: 339.1542, found: 339.1546.

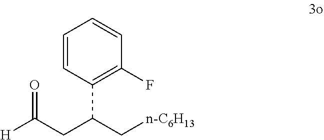

3-(2-fluorophenyl)decanal (30)

Following the General Procedure A, reaction time 28 h, 30 was obtained as a colorless oil in 58% yield. ¹H NMR (600 MHZ, CDCl₃) δ 9.68 (t, J=2.1 Hz, 1H), 7.21-7.16 (m, 2H), 7.12-7.07 (m, 1H), 7.04-6.98 (m, 1H), 3.49 (dq, J=8.8, 7.2 Hz, 1H), 2.75 (dd, J=7.3, 2.1 Hz, 2H), 1.70-1.63 (m, 2H), 1.28-1.09 (m, 10H), 0.85 (t, J=7.1 Hz, 3H). ¹³C NMR (151 MHZ, CDCl₃) δ 201.69, 160.91 (d, J_{CF}=244.9 Hz), 130.52 (d, J_{CF}=13.8 Hz), 128.85 (d, JCF=5.0 Hz), 128.00 (d, J_{CF}=8.8 Hz), 124.27 (d, J_{CF}=3.8 Hz), 115.66 (d, J_{CF}=23.1 Hz), 49.37 (d, J_{CF}=1.7 Hz), 35.22 (d, J_{CF}=1.7 Hz), 33.53, 31.77, 29.39, 29.12, 27.32, 22.61, 14.07. ¹⁹F NMR (376 MHZ, CDCl₃) δ −120.39. HRMS (ESI-TOF) Calcd for C₁₆H₂₃FNaO⁺ [M+Na]⁺: 273.1625, found: 273.1621.

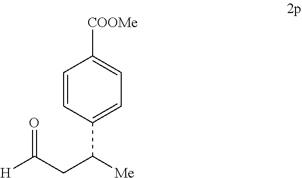 [SMILES: COC(=O)c1ccc([C@H](C)CC=O)cc1]

methyl 4-(4-oxobutan-2-yl)benzoate (2p)

Following the General Procedure A, reaction time 18 h, 2p was obtained as a colorless oil in 77% yield. ¹H NMR (600 MHZ, CDCl₃) δ 9.72 (t, J=1.8 Hz, 1H), 7.98 (d, J=8.4 Hz, 2H), 7.30 (d, J=8.3 Hz, 2H), 3.91 (s, 3H), 3.43 (h, J=7.0 Hz, 1H), 2.81-2.67 (m, 2H), 1.33 (d, J=7.0 Hz, 3H). ¹³C NMR (151 MHz, CDCl₃) δ 201.02, 166.92, 150.85, 130.07, 128.55, 126.86, 52.07, 51.44, 34.19, 21.91. Spectroscopic data for this compound is consistent with that shown in the literature.¹²

Physical Characterization of γ-arylation Products

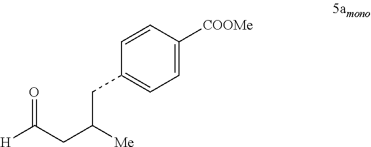

Methyl 4-(2-methyl-4-oxobutyl)benzoate (5amono)

Following the General Procedure B, 5amono was obtained as a colorless oil in 40% yield. ¹H NMR (600 MHz, CDCl₃) δ 9.73 (dd, J=2.3, 1.4 Hz, 1H), 7.97 (d, J=8.2 Hz, 2H), 7.23 (d, J=8.6 Hz, 2H), 3.91 (s, 3H), 2.68 (dd, J=13.4, 6.7 Hz, 1H), 2.59 (dd, J=13.4, 7.4 Hz, 1H), 2.45-2.37 (m, 2H), 2.32-2.26 (m, 1H), 0.97 (d, J=6.6 Hz, 3H). ¹³C NMR (151 MHZ, CDCl₃) δ 202.04, 167.04, 145.53, 129.73, 129.21, 128.28, 52.04, 50.23, 43.07, 29.97, 19.85. HRMS (ESI-TOF) Calcd for C₁₃H₁₆NaO₃⁺ [M+Na]⁺: 243.0991, found: 243.0992.

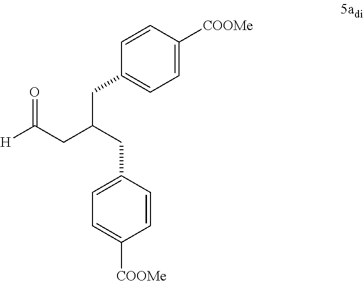

Dimethyl 4,4′-(2-(2-oxoethyl)propane-1,3-diyl)dibenzoate (5adi)

Following the General Procedure B, Sadi was obtained as a colorless oil in 32% yield. ¹H NMR (600 MHZ, CDCl₃) δ 9.61 (t, J=1.5 Hz, 1H), 7.97 (d, J=8.3 Hz, 4H), 7.22 (d, J=8.3 Hz, 4H), 3.91 (s, 6H), 2.73 (dd, J=13.2, 6.1 Hz, 2H), 2.69-2.59 (m, 3H), 2.35 (dd, J=5.9, 1.5 Hz, 2H). ¹³C NMR (151 MHz, CDCl₃) δ 201.32, 166.95, 145.07, 129.88, 129.22, 128.50, 52.08, 46.93, 40.30, 36.84. HRMS (ESI-TOF) Calcd for C₂₁H₂₃O₅⁺ [M+H]⁺: 55.1540, found: 355.1537.

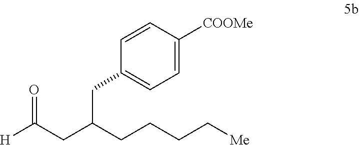

Methyl 4-(2-(2-oxoethyl)heptyl)benzoate (5b)

Following the General Procedure B, 5b was obtained as a colorless oil in 60% yield. ¹H NMR (600 MHz, CDCl₃) δ 9.68 (s, 1H), 7.96 (d, J=7.9 Hz, 2H), 7.23 (d, J=7.9 Hz, 2H), 3.90 (s, 3H), 2.75 (dd, J=13.6, 6.3 Hz, 1H), 2.58 (dd, J=13.6, 7.4 Hz, 1H), 2.33 (tq, J=12.2, 6.1 Hz, 3H), 1.28 (dtd, J=25.3, 13.4, 12.6, 6.6 Hz, 8H), 0.87 (t, J=7.1 Hz, 3H). ¹³C NMR (151 MHZ, CDCl₃) δ 202.33, 167.04, 145.73, 129.74, 129.27, 128.23, 52.03, 47.82, 40.59, 34.96, 33.92, 31.88, 26.41, 22.56, 14.03. HRMS (ESI-TOF) Calcd for C₁₈H₂₇O₂⁺ [M+H]⁺: 277.1804, found: 277.1805.

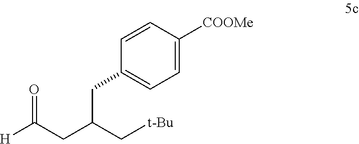

Methyl 4-(4,4-dimethyl-2-(2-oxoethyl)pentyl)benzoate (5c)

Following the General Procedure B, reaction time 36 h, 5c was obtained as a colorless oil in 72% yield. ¹H NMR (600 MHZ, CDCl₃) ¹H NMR (600 MHZ, CDCl₃) δ 9.67 (t, J=1.9 Hz, 1H), 7.96 (d, J=8.3 Hz, 2H), 7.25 (d, J=8.3 Hz, 2H), 3.91 (s, 3H), 2.73 (dd, J=13.5, 6.7 Hz, 1H), 2.61 (dd, J=13.5, 7.8 Hz, 1H), 2.43-2.35 (m, 2H), 2.32 (td, J=12.3, 6.5 Hz, 1H), 1.33 (dd, J=14.3, 4.5 Hz, 1H), 1.24 (dd, J=14.2, 5.3 Hz, 1H), 0.86 (s, 9H). ¹³C NMR (151 MHZ, CDCl₃) δ 202.27, 167.05, 145.89, 129.72, 129.37, 128.25, 52.03, 50.15, 47.39, 42.91, 31.44, 31.18, 29.81. HRMS (ESI-TOF) Calcd for C₁₇H₂₅O₃⁺ [M+H]⁺: 277.1804, found: 277.1799.

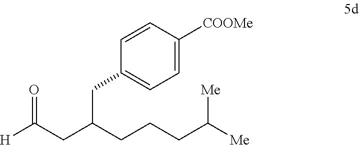

Methyl 4-(6-methyl-2-(2-oxoethyl)heptyl)benzoate (5d)

Following the General Procedure B, 5d was obtained as a colorless oil in 61% yield. ¹H NMR (600 MHz, CDCl₃) δ 9.69 (t, J=1.9 Hz, 1H), 7.96 (d, J=8.2 Hz, 2H), 7.23 (d, J=8.3 Hz, 2H), 3.91 (s, 3H), 2.75 (dd, J=13.6, 6.3 Hz, 1H), 2.59 (dd, J=13.6, 7.4 Hz, 1H), 2.39-2.2 5(m, 3H), 1.50 (dp, J=13.3, 6.6 Hz, 1H), 1.38-1.24 (m, 4H), 1.13 (qd, J=5.6, 4.6, 2.2 Hz, 2H), 0.85 (d, J=6.6 Hz, 6H). ¹³C NMR (151 MHZ, CDCl₃) δ 202.33, 167.05, 145.72, 129.75, 129.27, 128.24, 52.04, 47.84, 40.62, 38.96, 34.99, 34.22, 27.87, 24.51, 22.59, 22.56. HRMS (ESI-TOF) Calcd for C₁₈H₂₆NaO₃⁺ [M+Na]⁺: 313.1774, found: 313.1771.

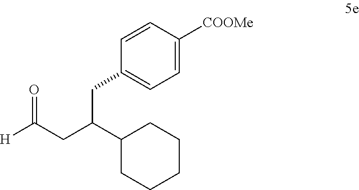

Methyl 4-(2-cyclohexyl-4-oxobutyl)benzoate (5e)

Following the General Procedure B, 5e was obtained as a colorless oil in 60% yield. ¹H NMR (600 MHZ, CDCl₃) δ 9.59 (t, J=1.9 Hz, 1H), 7.96 (d, J=8.2 Hz, 2H), 7.23 (d, J=8.2 Hz, 2H), 3.90 (s, 3H), 2.81 (dd, J=13.6, 6.0 Hz, 1H), 2.50 (dd, J=13.6, 8.4 Hz, 1H), 2.46-2.39 (m, 1H), 2.24 (dddd, J=17.1, 12.3, 6.6, 2.9 Hz, 2H), 1.82-1.62 (m, 5H), 1.35 (tq, J=11.8, 3.3 Hz, 1H), 1.23-1.02 (m, 5H). ¹³C NMR (151 MHz, CDCl₃) δ 202.46, 167.04, 146.26, 129.79, 129.22, 128.20, 52.02, 45.17, 40.43, 37.85, 30.25, 29.36, 26.56, 26.55. HRMS (ESI-TOF) Calcd for C₁₈H₂₄NaO₃⁺ [M+Na]⁺: 311.1617, found: 311.1615.

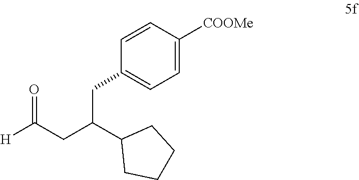

Methyl 4-(2-cyclopentyl-4-oxobutyl)benzoate (5f)

Following the General Procedure B, 5f was obtained as a colorless oil in 59% yield. ¹H NMR (600 MHz, CDCl₃) δ 9.61 (d, J=2.1 Hz, 1H), 7.96 (d, J=7.8 Hz, 2H), 7.24 (d, J=7.9 Hz, 2H), 3.90 (s, 3H), 2.89 (dd, J=13.7, 5.3 Hz, 1H), 2.57 (dd, J=13.7, 8.9 Hz, 1H), 2.35 (qd, J=17.0, 4.7 Hz, 2H), 2.25 (h, J=6.0 Hz, 1H), 1.84 (h, J=8.1 Hz, 2H), 1.77-1.71 (m, 1H), 1.64-1.48 (m, 4H), 1.24-1.14 (m, 2H). ¹³C NMR (151 MHZ, CDCl₃) δ 202.42, 167.03, 145.88, 129.77, 129.32, 128.23, 52.03, 46.29, 43.94, 40.12, 39.63, 30.77, 30.26, 25.34, 25.31. HRMS (ESI-TOF) Calcd for C₁₇H₂₂NaO₃⁺ [M+H]⁺: 297.1461, found: 297.1455.

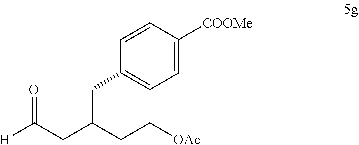

Methyl 4-(4-acetoxy-2-(2-oxoethyl)butyl)benzoate (5g)

Following the General Procedure B, 5g was obtained as a colorless oil in 56% yield. ¹H NMR (600 MHz, CDCl₃) ¹H NMR (600 MHz, CDCl₃) δ 9.70 (t, J=1.3 Hz, 1H), 7.97 (d, J=8.6 Hz, 2H), 7.24 (d, J=7.9 Hz, 2H), 4.17-4.07 (m, 2H), 3.91 (s, 3H), 2.77 (dd, J=13.5, 5.9 Hz, 1H), 2.66 (dd, J=13.5, 6.7 Hz, 1H), 2.51-2.34 (m, 3H), 2.05 (s, 3H), 1.76-1.64 (m, 2H). ¹³C NMR (151 MHz, CDCl₃) δ 201.35, 171.02, 166.96, 144.91, 129.86, 129.25, 128.50, 62.07, 52.08, 47.46, 40.38, 32.55, 31.93, 20.96. HRMS (ESI-TOF) Calcd for C₁₆H₂₀NaOs⁺ [M+Na]⁺: 315.1203, found: 315.1206.

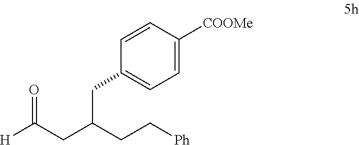

Methyl 4-(4-oxo-2-phenethylbutyl)benzoate (5h)

Following the General Procedure B, 5h was obtained as a colorless oil in 45% yield. ¹H NMR (600 MHz, CDCl₃) δ 9.68 (t, J=1.8 Hz, 1H), 7.98-7.94 (m, 2H), 7.27 (d, J=6.1 Hz, 2H), 7.23-7.20 (m, 2H), 7.20-7.17 (m, 1H), 7.14-7.11 (m, 2H), 3.91 (s, 3H), 2.82 (dd, J=13.6, 6.4 Hz, 1H), 2.71-2.60 (m, 3H), 2.45-2.32 (m, 3H), 1.75-1.62 (m, 2H). ¹³C NMR (151 MHz, CDCl₃) δ 201.94, 167.01, 145.35, 141.57, 129.80, 129.26, 128.48, 128.35, 128.27, 126.01, 52.05, 47.72, 40.40, 35.60, 34.45, 33.08. HRMS (ESI-TOF) Calcd for C₂₀H₂₃O₃⁺ [M+H]⁺: 311.1647, found: 311.1646.

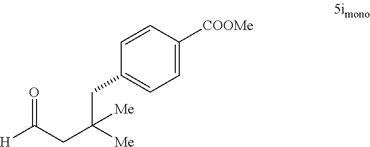

Methyl 4-(2,2-dimethyl-4-oxobutyl)benzoate (5iₘₒₙₒ)

Following the General Procedure B, 5iₘₒₙₒ was obtained as a colorless oil in 60% yield. ¹H NMR (600 MHZ, CDCl₃) δ 9.86 (t, J=2.8 Hz, 1H), 7.96 (d, J=8.3 Hz, 2H), 7.20 (d, J=8.4 Hz, 2H), 3.91 (s, 3H), 2.70 (s, 2H), 2.27 (d, J=2.8 Hz, 2H), 1.08 (s, 6H). ¹³C NMR (151 MHZ, CDCl₃) δ 202.96, 167.07, 143.47, 130.61, 129.26, 128.37, 54.31, 52.06, 48.57, 34.71, 27.39. HRMS (ESI-TOF) Calcd for C₁₄H₁₈NaO₃⁺ [M+Na]⁺: 257.1148, found: 257.1138.

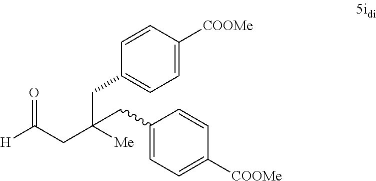

Dimethyl 4,4′-(2-methyl-2-(2-oxoethyl)propane-1,3-diyl)dibenzoate (5i_{di})

Following the General Procedure B, 5i_{di} was obtained as a colorless oil in 23% yield. ¹H NMR (600 MHz, CDCl₃) δ 9.81 (t, J=2.1 Hz, 1H), 7.96 (d, J=8.3 Hz, 4H), 7.20 (d, J=8.3 Hz, 4H), 3.91 (s, 6H), 2.92 (d, J=13.1 Hz, 2H), 2.77 (d, J=13.1 Hz, 2H), 2.23 (d, J=2.1 Hz, 2H), 1.03 (s, 3H). ¹³C NMR (151 MHz, CDCl₃) δ 202.46, 166.99, 143.05, 130.77, 129.38, 128.58, 52.09, 50.76, 46.54, 38.49, 24.56. HRMS (ESI-TOF) Calcd for C₂₂H₂₅O₃⁺ [M+H]⁺: 369.1697, found: 369.1691.

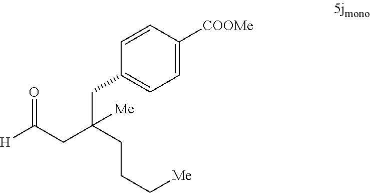

Methyl 4-(2-methyl-2-(2-oxoethyl)hexyl)benzoate (5jₘₒₙₒ)

Following the General Procedure B, 5jₘₒₙₒ was obtained as a colorless oil in 44% yield. 1H NMR (600 MHZ, CDCl₃) δ 9.84 (t, J=2.8 Hz, 1H), 7.95 (d, J=8.3 Hz, 2H), 7.20 (d, J=8.3 Hz, 2H), 3.91 (s, 3H), 2.73 (q, J=13.2 Hz, 2H), 2.32-2.20 (m, 2H), 1.44-1.28 (m, 6H), 1.04 (s, 3H), 0.92 (t, J=7.1 Hz, 3H). ¹³C NMR (151 MHZ, CDCl₃) δ 203.16, 167.08, 143.53, 130.68, 129.26, 128.32, 52.05, 51.97, 46.23, 39.62, 37.47, 26.05, 25.05, 23.28, 14.10. HRMS (ESI-TOF) Calcd for C₁₇H₂₄NaO₃⁺ [M+Na]⁺: 299.1617, found: 299.1613.

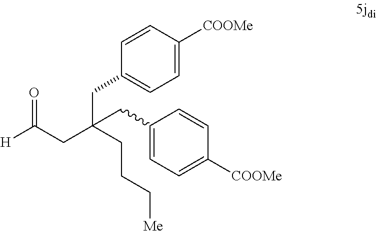

Dimethyl 4,4′-(2-butyl-2-(2-oxoethyl)propane-1,3-diyl)dibenzoate (5j_{di})

Following the General Procedure B, 5j_{di} was obtained as a colorless oil in 16% yield. ¹H NMR (600 MHZ, CDCl₃) δ 9.73-9.69 (m, 1H), 7.96 (d, J=8.3 Hz, 4H), 7.19 (d, J=8.3 Hz, 4H), 3.91 (s, 6H), 2.93-2.84 (m, 4H), 2.20 (d, J=2.0 Hz, 2H), 1.52-1.44 (m, 2H), 1.33 (h, J=6.8 Hz, 4H), 0.95 (t, J=7.3 Hz, 3H). ¹³C NMR (151 MHz, CDCl₃) δ 202.49, 166.97, 143.28, 130.69, 129.45, 128.55, 52.10, 49.49, 43.06, 41.47, 35.42, 25.88, 23.10, 14.15. HRMS (ESI-TOF) Calcd for C₂₅H₃₁O₅⁺ [M+H]⁺: 411.2166, found: 411.2168.

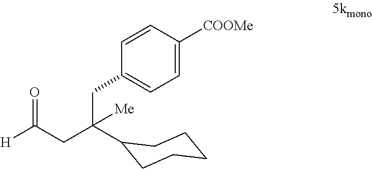

Methyl 4-(2-cyclohexyl-2-methyl-4-oxobutyl)benzoate (5kₘₒₙₒ)

Following the General Procedure B, 5kₘₒₙₒ was obtained as a colorless oil in 37% yield. ¹H NMR (600 MHZ, CDCl₃) δ 9.79 (t, J=2.8 Hz, 1H), 7.95 (d, J=8.2 Hz, 2H), 7.20 (d, J=8.2 Hz, 2H), 3.91 (s, 3H), 2.82-2.72 (m, 2H), 2.32 (dd, J=15.4, 3.1 Hz, 1H), 2.19 (dd, J=15.4, 2.5 Hz, 1H), 1.89-1.78 (m, 4H), 1.69 (d, J=12.4 Hz, 1H), 1.36 (tt, J=11.7, 2.8 Hz, 1H), 1.23-1.05 (m, 5H), 1.02 (s, 3H). ¹³C NMR (151 MHZ, CDCl₃) δ 203.50, 167.07, 143.96, 130.81, 129.24, 128.28, 52.05, 50.31, 45.30, 43.42, 40.32, 27.57, 27.27, 26.91, 26.53, 22.63. HRMS (ESI-TOF) Calcd for C₁₉H₂₆NaO₃⁺ [M+Na]⁺: 325.1774, found: 325.1766.

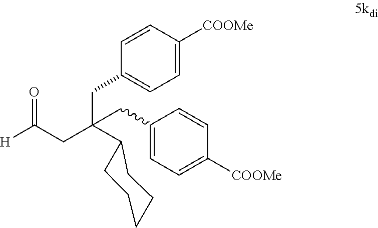

Dimethyl 4,4′-(2-cyclohexyl-2-(2-oxoethyl)propane-1,3-diyl)dibenzoate (5k_{di})

Following the General Procedure B, 5k_{di} was obtained as a colorless oil in 9% yield. ¹H NMR (600 MHZ, CDCl₃) δ 9.09 (t, J=2.3 Hz, 1H), 7.95 (d, J=8.4 Hz, 4H), 7.19 (d, J=8.4 Hz, 4H), 3.91 (s, 6H), 3.00 (d, J=13.4 Hz, 2H), 2.78 (d, J=13.4 Hz, 2H), 2.31 (d, J=2.4 Hz, 2H), 1.86 (dd, J=17.8, 10.7 Hz, 4H), 1.69 (dd, J=32.4, 10.2 Hz, 2H), 1.27-1.16 (m, 5H). ¹³C NMR (151 MHZ, CDCl₃) δ 202.45, 166.91, 143.36, 131.00, 129.52, 128.65, 52.10, 48.61, 44.03, 42.31, 41.80, 27.54, 26.89, 26.52. HRMS (ESI-TOF) Calcd for C₂₇H₃₃O₅⁺ [M+H]⁺: 437.2323, found: 437.2328.

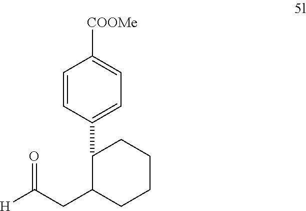 [SMILES: COC(=O)c1ccc([C@@H]2CCCCC2CC=O)cc1]

Methyl 4-(2-(2-oxoethyl)cyclohexyl)benzoate (5l)

Following the General Procedure B, 5l was obtained as a colorless oil in 20% yield. ¹H NMR (600 MHZ, CDCl₃) ¹H NMR (600 MHz, CDCl₃) δ 9.50 (dt, J=2.3, 1.1 Hz, 1H), 7.97 (d, J=8.6 Hz, 2H), 7.24 (d, J=8.0 Hz, 2H), 3.90 (s, 3H), 2.34 (td, J=11.5, 3.5 Hz, 1H), 2.22-2.11 (m, 2H), 2.10-2.04 (m, 1H), 1.96-1.90 (m, 1H), 1.88-1.78 (m, 3H), 1.53-1.48 (m, 1H), 1.48-1.32 (m, 2H), 1.22-1.10 (m, 1H). ¹³C NMR (151 MHZ, CDCl₃) ¹³C NMR (151 MHZ, CDCl₃) ¹³C NMR (151 MHz, CDCl₃) δ 202.08, 166.97, 150.75, 130.04, 128.52, 127.62, 52.03, 50.36, 49.02, 37.75, 35.25, 33.18, 26.45, 26.11. HRMS (ESI-TOF) Calcd for C₁₆H₂₀NaO₃⁺ [M+Na]⁺: 283.1304, found: 283.1303.

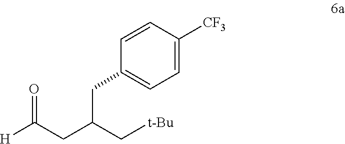

5,5-dimethyl-3-(4-(trifluoromethyl)benzyl)hexanal (6a)

Following the General Procedure B, reaction time 36 h, 6a was obtained as a colorless oil in 64% yield. ¹H NMR (600 MHZ, CDCl₃) δ 9.69 (t, J=1.9 Hz, 1H), 7.54 (d, J=7.7 Hz, 2H), 7.32-7.27 (m, 2H), 2.74 (dd, J=13.5, 6.6 Hz, 1H), 2.61 (dd, J=13.5, 7.8 Hz, 1H), 2.40 (dt, J=6.1, 1.8 Hz, 2H), 2.34-2.28 (m, 1H), 1.33 (dd, J=14.3, 4.6 Hz, 1H), 1.25 (dd, J=14.3, 5.3 Hz, 1H), 0.87 (s, 9H). ¹³C NMR (151 MHz, CDCl₃) ¹³C NMR (151 MHz, CDCl₃) δ 202.20, 144.49, 129.61, 128.53 (q, JCF=96.3 Hz), 125.30 (q, J_{CF}=3.9 Hz), 124.27 (q, J_{CF}=272.0 Hz), 50.00, 47.36, 42.63, 31.38, 31.19, 29.81. 19F NMR (376 MHz, CDCl₃) 8-65.01. HRMS (ESI-TOF) Calcd for C₁₆H₂₁F₃NaO⁺ [M+Na]⁺: 309.1436, found: 309.1440.

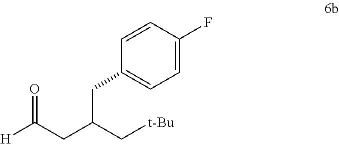

3-(4-fluorobenzyl)-5,5-dimethylhexanal (6b)

Following the General Procedure B, reaction time 36 h, 6b was obtained as a colorless oil in 57% yield. ¹H NMR (600 MHZ, CDCl₃) ¹H NMR (600 MHZ, CDCl₃) δ 9.66 (t, J=2.0 Hz, 1H), 7.12 (dd, J=8.5, 5.5 Hz, 2H), 6.97 (t, J=8.7 Hz, 2H), 2.67 (dd, J=13.7, 6.5 Hz, 1H), 2.50 (dd, J=13.6, 8.0 Hz, 1H), 2.41-2.34 (m, 2H), 2.28-2.21 (m, 1H), 1.33 (dd, J=14.3, 4.6 Hz, 1H), 1.23 (dd, J=14.2, 5.2 Hz, 1H), 0.87 (s, 9H). ¹³C NMR (151 MHZ, CDCl₃) δ 202.51, 161.49 (d, J_{CF}=244.3 Hz), 135.95 (d, J_{CF}=2.8 Hz), 130.66 (d, J_{CF}=7.7 Hz), 115.15 (d, J_{CF}=21.1 Hz), 50.10, 47.41, 42.15, 31.77, 31.21, 29.82. 19F NMR (376 MHZ, CDCl₃) δ −119.65. HRMS (ESI-TOF) Calcd for C₁₅H₂₁FNaO⁺ [M+Na]⁺: 259.1468, found: 259.1467.

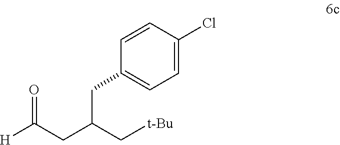

3-(4-chlorobenzyl)-5,5-dimethylhexanal (6c)

Following the General Procedure B, reaction time 36 h, 6c was obtained as a colorless oil in 64% yield. ¹H NMR (600 MHZ, CDCl₃) δ 9.66 (t, J=2.0 Hz, 1H), 7.25 (d, J=8.7 Hz, 2H), 7.10 (d, J=8.4 Hz, 2H), 2.66 (dd, J=13.6, 6.5 Hz, 1H), 2.50 (dd, J=13.6, 8.0 Hz, 1H), 2.38 (dd, J=6.1, 2.1 Hz, 2H), 2.26 (ddt, J=12.8, 7.9, 3.0 Hz, 1H), 1.32 (dd, J=14.3, 4.6 Hz, 1H), 1.23 (dd, J=14.3, 5.3 Hz, 1H), 0.87 (s, 9H). ¹³C NMR (151 MHz, CDCl₃) δ 202.40, 138.77, 132.01, 130.64, 128.49, 50.04, 47.40, 42.25, 31.56, 31.19, 29.82. HRMS (ESI-TOF) Calcd for C₁₅H₂₁ClNaO⁺ [M+Na]⁺: 275.1173, found: 275.1172.

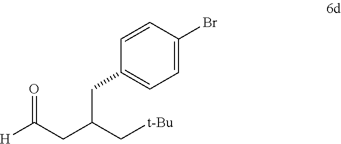

3-(4-bromobenzyl)-5,5-dimethylhexanal (6d)

Following the General Procedure B, reaction time 36 h, 6d was obtained as a colorless oil in 63% yield. ¹H NMR (600 MHz, CDCl₃) δ 9.67 (t, J=2.0 Hz, 1H), 7.40 (d, J=8.3 Hz, 2H), 7.05 (d, J=8.4 Hz, 2H), 2.65 (dd, J=13.6, 6.5 Hz, 1H), 2.49 (dd, J=13.6, 8.0 Hz, 1H), 2.38 (dd, J=6.4, 1.9 Hz, 2H), 2.26 (dddd, J=12.9, 7.9, 6.3, 3.2 Hz, 1H), 1.32 (dd, J=14.2, 4.6 Hz, 1H), 1.23 (dd, J=14.3, 5.3 Hz, 1H), 0.87 (s, 9H). ¹³C NMR (151 MHz, CDCl₃) δ 202.39, 139.29, 131.44, 131.04, 120.04, 50.02, 47.39, 42.30, 31.49, 31.19, 29.83. HRMS (ESI-TOF) Calcd for C₁₅H₂₁BrNaO⁺ [M+Na]⁺: 319.0668, found: 319.0669.

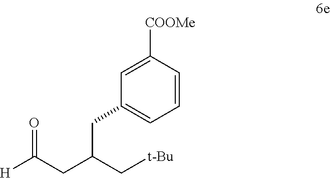

Methyl 3-(4,4-dimethyl-2-(2-oxoethyl)pentyl)benzoate (6e)

Following the General Procedure B, reaction time 36 h, 6e was obtained as a colorless oil in 69% yield. ¹H NMR (600 MHz, CDCl₃) δ 9.67 (t, J=2.0 Hz, 1H), 7.91-7.86 (m, 1H), 7.84 (s, 1H), 7.39-7.35 (m, 2H), 3.92 (s, 3H), 2.76 (dd, J=13.6, 6.5 Hz, 1H), 2.59 (dd, J=13.6, 8.0 Hz, 1H), 2.41-2.36 (m, 2H), 2.36-2.27 (m, 1H), 1.34 (dd, J=14.3, 4.6 Hz, 1H), 1.25 (dd, J=14.3, 5.2 Hz, 1H), 0.87 (s, 9H). ¹³C NMR (151 MHz, CDCl₃) δ 202.39, 167.16, 140.69, 133.96, 130.31, 130.25, 128.47, 127.59, 52.14, 50.05, 47.42, 42.71, 31.55, 31.21, 29.84. HRMS (ESI-TOF) Calcd for C₁₇H₂₄NaO₃⁺ [M+Na]⁺: 299.1617, found: 299.1621.

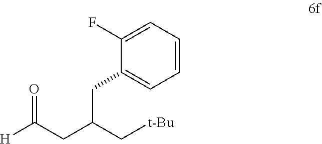

3-(2-fluorobenzyl)-5,5-dimethylhexanal (6f)

Following the General Procedure B, reaction time 36 h, 6f was obtained as a colorless oil in 61% yield. ¹H NMR (600 MHZ, CDCl₃) ¹H NMR (600 MHZ, CDCl₃) δ 9.65 (t, J=2.0 Hz, 1H), 7.22-7.14 (m, 2H), 7.06 (td, J=7.5, 1.2 Hz, 1H), 7.02 (ddd, J=9.7, 8.2, 1.2 Hz, 1H), 2.80 (dd, J=13.5, 5.4 Hz, 1H), 2.54 (dd, J=13.5, 7.6 Hz, 1H), 2.42-2.30 (m, 3H), 1.34 (dd, J=14.2, 4.4 Hz, 1H), 1.27 (dd, J=14.2, 4.3 Hz, 1H), 0.89 (s, 9H). ¹³C NMR (151 MHZ, CDCl₃) δ 202.65, 161.38 (d, J_{CF}=244.8 Hz), 131.71 (d, J_{CF}=5.0 Hz), 128.11 (d, J_{CF}=8.2 Hz), 127.18 (d, J_{CF}=15.9 Hz), 123.96 (d, J_{CF}=3.4 Hz), 115.37 (d, J_{CF}=22.5 Hz), 50.11, 47.85, 36.26, 31.19, 30.63, 29.74. 19F NMR (376 MHz, CDCl₃) 8-120.03. HRMS (ESI-TOF) Calcd for C₁₅H₂₁FNaO⁺ [M+H]⁺: 259.1468, found: 259.1470.

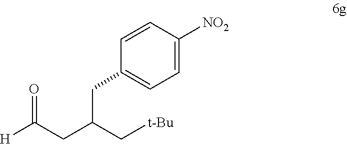

5,5-dimethyl-3-(4-nitrobenzyl)hexanal (6g)

Following the General Procedure B, reaction time 36 h, 6g was obtained as a colorless oil in 70% yield. ¹H NMR (600 MHZ, CDCl₃) δ 9.72 (t, J=1.7 Hz, 1H), 8.16 (d, J=8.7 Hz, 2H), 7.35 (d, J=8.6 Hz, 2H), 2.76-2.65 (m, 2H), 2.51-2.39 (m, 2H), 2.34 (dtdd, J=7.2, 5.8, 4.8, 2.9 Hz, 1H), 1.33-1.24 (m, 2H), 0.85 (s, 9H). ¹³C NMR (151 MHZ, CDCl₃) δ 201.78, 148.34, 146.63, 130.14, 123.65, 50.18, 47.24, 42.63, 31.25, 31.14, 29.76. HRMS (ESI-TOF) Calcd for C₁₅H22NO₃⁺ [M+H]⁺: 264.1594, found: 264.1592.

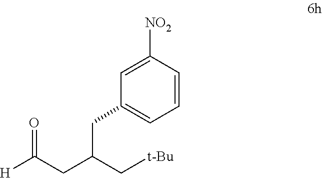

5,5-dimethyl-3-(3-nitrobenzyl)hexanal (6h)

Following the General Procedure B, reaction time 36 h, 6h was obtained as a colorless oil in 71% yield. ¹H NMR (600 MHZ, CDCl₃) δ 9.72 (t, J=1.7 Hz, 1H), 8.08 (ddd, J=8.1, 2.3, 1.1Hz, 1H), 8.05 (t, J=2.0 Hz, 1H), 7.56-7.50 (m, 1H), 7.47 (t, J=7.8 Hz, 1H), 2.77-2.66 (m, 2H), 2.50-2.39 (m, 2H), 2.37-2.30 (m, 1H), 1.30 (qd, J=14.3, 4.9 Hz, 2H), 0.86 (s, 9H). ¹³C NMR (151 MHz, CDCl₃) δ 201.83, 148.28, 142.45, 135.60, 129.30, 124.01, 121.51, 50.09, 47.19, 42.41, 31.25, 31.17, 29.78. HRMS (ESI-TOF) Calcd for C₁₅H₂₂NO₃⁺ [M+H]⁺: 264.1594, found: 264.1596.

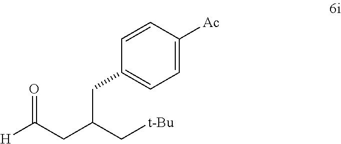

3-(4-acetylbenzyl)-5,5-dimethylhexanal (6i)

Following the General Procedure B, reaction time 36 h, 6i was obtained as a colorless oil in 59% yield. ¹H NMR (600 MHz, CDCl₃) δ 9.68 (t, J=1.9 Hz, 1H), 7.89 (d, J=8.3 Hz, 2H), 7.27 (d, J=8.0 Hz, 2H), 2.74 (dd, J=13.5, 6.6 Hz, 1H), 2.64-2.59 (m, 1H), 2.59 (s, 3H), 2.40 (dt, J=6.0, 1.5 Hz, 2H), 2.36-2.29 (m, 1H), 1.34 (dd, J=14.3, 4.5 Hz, 1H), 1.25 (dd, J=14.3, 5.3 Hz, 1H), 0.87 (s, 9H). ¹³C NMR (151 MHz, CDCl₃) δ 202.25, 197.82, 146.17, 135.41, 129.55, 128.54, 50.12, 47.44, 42.88, 31.40, 31.19, 29.82, 26.58. HRMS (ESI-TOF) Calcd for C₁₇H₂₅O₂⁺ [M+H]⁺: 261.1849, found: 261.1853.

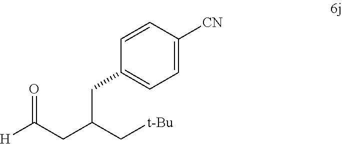

4-(4,4-dimethyl-2-(2-oxoethyl)pentyl)benzonitrile (6j)

Following the General Procedure B, reaction time 36 h, 6j was obtained as a colorless oil in 48% yield. ¹H NMR (600 MHz, CDCl₃) δ 9.70 (t, J=1.7 Hz, 1H), 7.59 (d, J=8.4 Hz, 2H), 7.29 (d, J=8.5 Hz, 2H), 2.72-2.61 (m, 2H), 2.50-2.37 (m, 2H), 2.30 (qtd, J=7.1, 5.6, 4.4 Hz, 1H), 1.31-1.23 (m, 2H), 0.85 (s, 9H). ¹³C NMR (151 MHz, CDCl₃) δ 201.87, 146.12, 132.20, 130.11, 118.94, 110.21, 50.14, 47.24, 42.91, 31.22, 31.14, 29.75. HRMS (ESI-TOF) Calcd for C₁₆H₂₂NO⁺ [M+H]⁺: 244.1696, found: 244.1704.

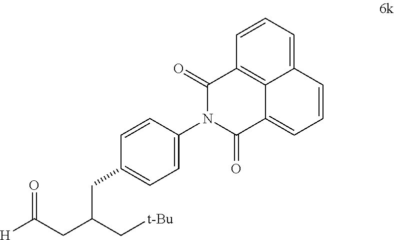

3-(4-(1,3-dioxo-1H-benzo[de]isoquinolin-2 (3H)-yl)benzyl)-5,5-dimethylhexanal (6k)

Following the General Procedure B, reaction time 36 h, 6k was obtained as white solid in 56% yield. ¹H NMR (600 MHZ, CDCl₃) δ 9.73 (dd, J=2.3, 1.7 Hz, 1H), 8.65 (dd, J=7.2, 1.1 Hz, 2H), 8.28 (dd, J=8.3, 1.1 Hz, 2H), 7.80 (dd, J=8.2, 7.3 Hz, 2H), 7.36 (d, J=8.3 Hz, 2H), 7.25 (d, J=8.3 Hz, 2H), 2.86 (dd, J=13.5, 5.6 Hz, 1H), 2.62 (dd, J=13.5, 8.2 Hz, 1H), 2.50 (ddd, J=16.2, 5.8, 1.8 Hz, 1H), 2.44-2.33 (m, 2H), 1.41 (dd, J=14.3, 5.0 Hz, 1H), 1.32-1.29 (m, 1H), 0.93 (s, 9H). ¹³C NMR (151 MHz, CDCl₃) δ 202.79, 164.42, 140.90, 134.28, 133.54, 131.77, 131.62, 130.23, 128.57, 128.56, 127.05, 122.86, 49.82, 47.54, 42.58, 31.58, 31.31, 29.90. HRMS (ESI-TOF) Calcd for C₂₇H₂₈NO₃⁺ [M+H]⁺: 414.2064, found: 414.2064.

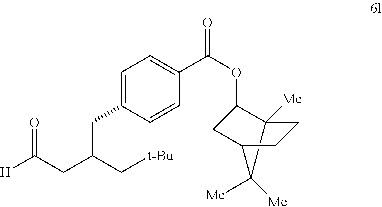

(2R)-1,7,7-trimethylbicyclo[2.2.1]heptan-2-yl 4-(4,4-dimethyl-2-(2-oxoethyl)pentyl)benzoate (61)

Following the General Procedure B, reaction time 36 h, 61 was obtained as a colorless oil in 65% yield. ¹H NMR (600 MHz, CDCl₃) δ 9.67 (t, J=1.9 Hz, 1H), 7.98 (d, J=8.2 Hz, 2H), 7.25 (d, J=8.2 Hz, 2H), 5.10 (dddd, J=10.0, 3.5, 2.1, 1.3 Hz, 1H), 2.77 (dd, J=13.5, 6.3 Hz, 1H), 2.60 (dd, J=13.5, 8.0 Hz, 1H), 2.50-2.44 (m, 1H), 2.39 (dd, J=6.4, 1.9 Hz, 2H), 2.37-2.29 (m, 1H), 2.13 (ddd, J=12.9, 9.5, 4.5 Hz, 1H), 1.86-1.77 (m, 1H), 1.74 (t, J=4.5 Hz, 1H), 1.41 (dddd, J=14.3, 12.2, 4.5, 2.2 Hz, 1H), 1.37-1.28 (m, 2H), 1.26 (dd, J=14.3, 5.2 Hz, 1H), 1.12 (ddd, J=13.8, 3.5, 1.5 Hz, 1H), 0.97 (s, 3H), 0.92 (d, J=3.1 Hz, 6H), 0.88 (d, J=0.8 Hz, 9H). ¹³C NMR (151 MHZ, CDCl₃) δ 202.32, 166.75, 145.66, 129.67, 129.31, 128.99, 80.43, 50.00, 49.09, 47.88, 47.49, 45.00, 42.90, 36.92, 31.48, 31.21, 29.86, 28.10, 27.40, 19.74, 18.93, 13.63. HRMS (ESI-TOF) Calcd for C₂₆H₃₈NaO₃⁺ [M+Na]⁺: 421.2713, found: 421.2712.

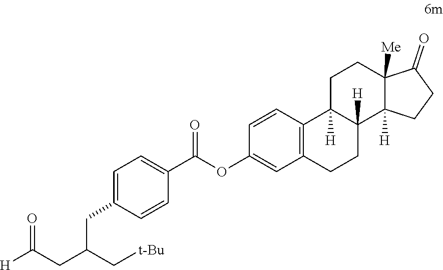

(8R,9S,13S,14S)-13-methyl-17-oxo-7,8,9,11,12,13,14,15,16,17-decahydro-6H-cyclopenta[a]phenanthren-3-yl 4-(4,4-dimethyl-2-(2-oxoethyl) pentyl)benzoate (6m)

Following the General Procedure B, reaction time 36 h, 6m was obtained as a white solid in 57% yield. ¹H NMR (600 MHZ, CDCl₃) δ 9.69 (t, J=1.9 Hz, 1H), 8.11 (d, J=8.2 Hz, 2H), 7.35-7.29 (m, 3H), 6.98 (dd, J=8.5, 2.6 Hz, 1H), 6.96-6.93 (m, 1H), 2.97-2.90 (m, 2H), 2.77 (dd, J=13.4, 6.7 Hz, 1H), 2.65 (dd, J=13.4, 7.7 Hz, 1H), 2.52 (ddd, J=19.1, 8.8, 1.0 Hz, 1H), 2.46-2.39 (m, 3H), 2.33 (ddd, J=15.5, 9.7, 4.8 Hz, 2H), 2.16 (dt, J=19.0, 9.0 Hz, 1H), 2.09-1.96 (m, 3H), 1.65-1.48 (m, 6H), 1.36 (dd, J=14.3, 4.4 Hz, 1H), 1.27 (dd, J=14.2, 5.5 Hz, 1H), 0.93 (s, 3H), 0.88 (s, 9H). ¹³C NMR (151 MHz, CDCl₃) δ 220.80, 202.18, 165.37, 148.87, 146.64, 138.07, 137.40, 130.30, 129.55, 127.72, 126.46, 121.74, 118.90, 50.46, 50.17, 47.97, 47.42, 44.20, 42.97, 38.04, 35.87, 31.57, 31.43, 31.19, 29.83, 29.44, 26.37, 25.79, 21.61, 13.85. HRMS (ESI-TOF) Calcd for C₃₄H₄₃O₄⁺ [M+H]⁺: 515.3156, found: 515.3155.

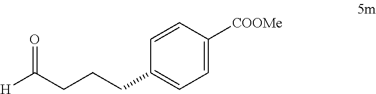

methyl 4-(4-oxobutyl)benzoate (5m)

Following the General Procedure B, with slightly modification of solvent volume to 0.65 mL and L8 loading to 80 mol %, 5m was obtained as a colorless oil in 62% yield (mixed with 1/9 2p). ¹H NMR (600 MHZ, CDCl₃) δ 9.77 (t, J=1.5 Hz, 1H), 7.97 (d, J=8.2 Hz, 2H), 7.25 (d, J=8.2 Hz, 2H), 3.91 (s, 3H), 2.73-2.70 (m, 2H), 2.47 (td, J=7.2, 1.5 Hz, 2H), 1.98 (p, J=7.3 Hz, 2H). ¹³C NMR (151 MHZ, CDCl₃) δ 201.89, 167.05, 146.74, 129.84, 128.49, 128.16, 52.04, 43.03, 34.99, 23.26. Spectroscopic data for this compound is consistent with that shown in the literature.¹³

### 6. Deuterium Incorporation Experiments

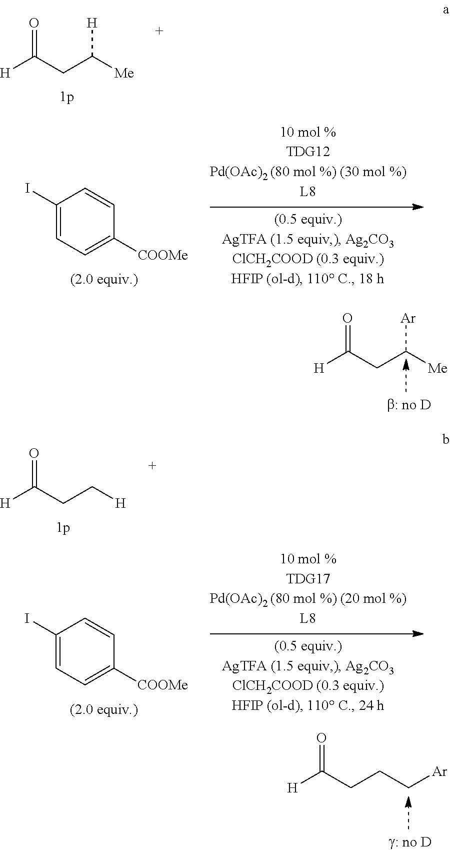

### Deuterium Incorporation Experiments a:

In air, to an oven-dried reaction tube (10 mL) equipped with a magnetic stir bar was added Pd(OAc)₂ (0.01 mmol, 10 mol %), transient directing groups (TDG12, 0.03 mmol, 30 mol %), ligand (L8, 0.08 mmol, 80 mol %), ArI (0.2 mmol, 2.0 equiv), AgTFA (0.15 mmol, 1.5 equiv), Ag₂CO₃ (0.05 mmol, 0.5 equiv), and solvent (HFIP-d₁, 0.75 mL and 0.02 mmol of ClCH₂COOD), followed by the aldehyde substrate 1p (0.1 mmol, 1.0 equiv). The tube was sealed and stirred at room temperature for 20 min before heating to 110° C. for 18 h under vigorous stirring. Upon completion, the reaction mixture was cooled to room temperature and the dark brown suspension was diluted with 2 mL of ethyl acetate and was passed through a pad of Celite and washed with ethyl acetate (1.0 mL×3). The crude reaction mixture was purified on silica gel using hexanes/EtOAc as the eluent to to get the arylation product (no deuteration at the B position). This result indicated that the β-methylene C(sp³)—H activation was not reversible under the reaction conditions.

### Deuterium Incorporation Experiments b:

In air, to an oven-dried reaction tube (10 mL) equipped with a magnetic stir bar was added Pd(OAc)₂ (0.01 mmol, 10 mol %), transient directing groups (TDG7, 0.02 mmol, 20 mol %), ligand (L8, 0.08 mmol, 80 mol %), ArI (0.2 mmol, 2.0 equiv), AgTFA (0.15 mmol, 1.5 equiv), Ag₂CO₃ (0.05 mmol, 0.5 equiv), and solvent (HFIP-d₁, 0.65 mL and 0.03 mmol of ClCH₂COOD), followed by the aldehyde substrate 1p (0.1 mmol, 1.0 equiv). The tube was sealed and stirred at room temperature for 20 min before heating to 110° C. for 24 h under vigorous stirring. Upon completion, the reaction mixture was cooled to room temperature and the dark brown suspension was diluted with 2 mL of ethyl acetate and was passed through a pad of Celite and washed with ethyl acetate (1.0 mL×3). The crude reaction mixture was purified on silica gel using hexanes/EtOAc as the eluent to to get the arylation product (no deuteration at the γ position). This result indicated that the γ-C(sp³)—H activation was not reversible under the reaction conditions.

### 7. Kinetic Isotope Effect (KIE) Experiments

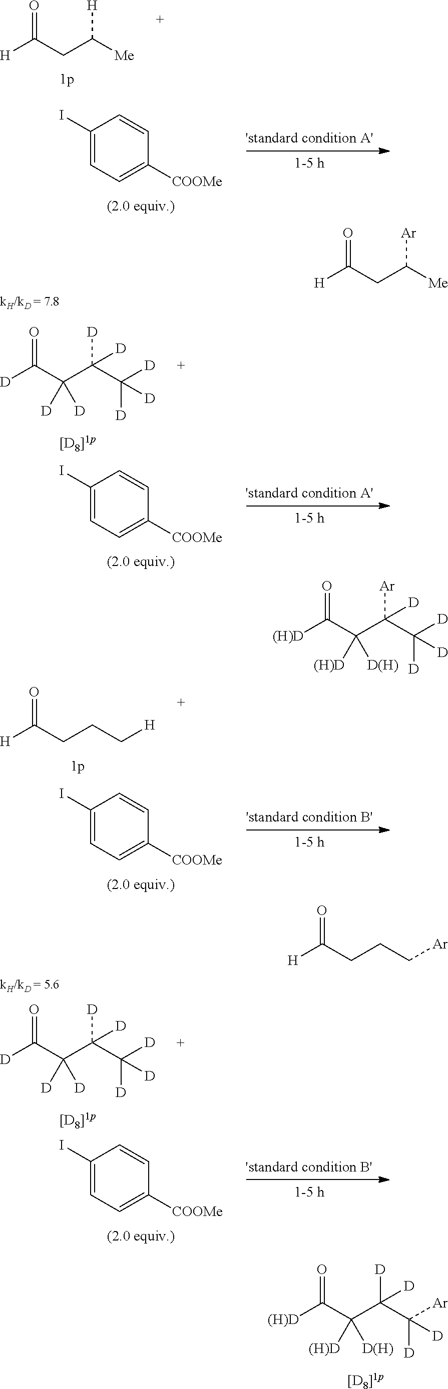

### Standard Condition A:

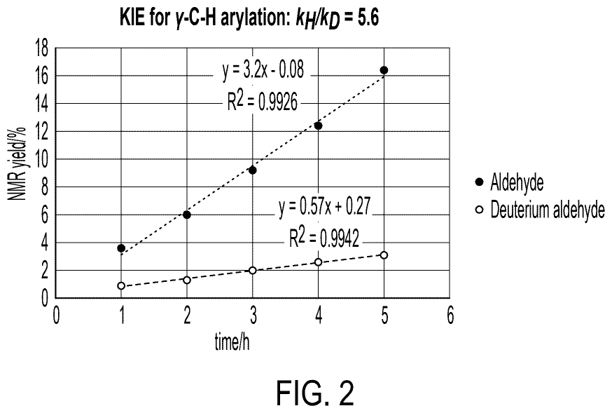

In air, to an oven-dried reaction tube (10 mL) equipped with a magnetic stir bar was added Pd(OAc)₂ (0.01 mmol, 10 mol %), transient directing groups (TDG12, 0.03 mmol, 30 mol %), ligand (L8, 0.08 mmol, 80 mol %), ArI (0.2 mmol, 2.0 equiv), AgTFA (0.15 mmol, 1.5 equiv), Ag2CO3 (0.05 mmol, 0.5 equiv), and solvent (HFIP, 0.75 mL and 0.02 mmol of CICH2COOH), followed by the aldehyde substrate 1p or 1p-d8 (0.1 mmol, 1.0 equiv). The tube was sealed and stirred at room temperature for 20 min before heating to 110° C. for 1-5 h under vigorous stirring. The reaction was quenched by freezing the vial in a dry ice-acetone bath at the indicated time, after then the mixture was diluted with 2 mL of ethyl acetate and was passed through a pad of Celite and washed with ethyl acetate (1.0 mL×3). The yield of β-arylation product was determined by ¹H NMR using CH₂Br₂ as internal standard. The result indicated that the KIE were 7.8 for the B position respectively (See FIG. 2).

### Standard Condition B:

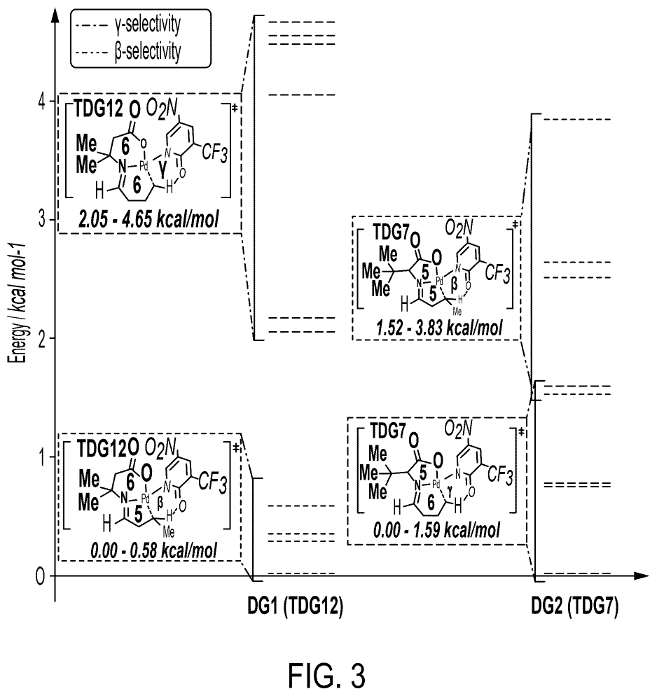

In air, to an oven-dried reaction tube (10 mL) equipped with a magnetic stir bar was added Pd(OAc)₂ (0.01 mmol, 10 mol %), transient directing groups (TDG7, 0.02 mmol, 20 mol %), ligand (L8, 0.08 mmol, 80 mol %), ArI (0.2 mmol, 2.0 equiv), AgTFA (0.15 mmol, 1.5 equiv), Ag2CO3 (0.05 mmol, 0.5 equiv), and solvent (HFIP, 0.65 mL and 0.03 mmol of ClCH₂COOH), followed by the aldehyde substrate 1p or 1p-d8 (0.1 mmol, 1.0 equiv). The tube was sealed and stirred at room temperature for 20 min before heating to 110° C. for 1-5 h under vigorous stirring. The reaction was quenched by freezing the vial in a dry ice-acetone bath at the indicated time, after then the mixture was diluted with 2 mL of ethyl acetate and was passed through a pad of Celite and washed with ethyl acetate (1.0 mL×3). The yield of y-arylation product was determined by ¹H NMR using CH₂Br₂ as internal standard. The result indicated that the KIE were 5.6 for the y position respectively. (see FIG. 3)

### 8. Computational Data and Analysis

### A. Computational Methods

DFT calculations were performed with Gaussian 16 (Rev. B.01) suite of quantum chemical programs¹⁴ with a pruned (99,590)-quadrature integration grid. Geometry optimizations were carried out with tight convergence thresholds using the B3LYP¹⁵⁻¹⁸ density functional, combined with Grimme's D3 empirical dispersion correction¹⁹ with Becke-Johnson damping.²⁰ The SDD basis set was used on Pd with Stuttgart-Dresden ECPs,21 and a split-valence 6-31G(d,p) basis set²²⁻²⁴ was used for all other atoms, augmented with diffuse functions on O atoms. Frequency analysis was carried out for all calculated transition state (TS) structures at the optimization level of theory to verify them as saddle points by the presence of precisely one imaginary vibrational frequency corresponding to the appropriate reactive vibrational mode. Single point electronic energies were computed at the PBE0²⁵-D3_{BJ}/6-311++G(2d,p)/SDD(Pd) level. Bulk solvation effects of HFIP were implemented implicitly as a generic solvent (ϵ=17.8, n²=1.629452)^{26,27} for all calculations using the integral equation formalism polarizable continuum (IEF-PCM) solvation model.²⁸ The quasi-RRHO (rigid-rotor-harmonic-oscillator) approximation was applied to vibrational entropies, as proposed by Grimme,²⁹ switching to a free rotor description of vibrational modes below 100 cm⁻¹, with a smooth damping function applied to interpolate between the two limiting descriptions at the cut-off frequency (GoodVibes v.3.0.1).³⁰ Enthalpies, quasi-harmonic Gibbs free energies, and Boltzmann population factors were evaluated at the reaction temperature (as indicated) and adjusted to the standard state concentration of 1 mol·dm⁻³.³⁰ All presented structures were visualized with CylView 1.06.³¹

### B. Energy Span of TS Ensembles Based on the Transient Directing Group (TDG)

See FIG. 3: Relative quasi-harmonic Gibbs free energies (Δqh-G₃₈₃) are plotted in kcal/mol for the C(sp³)—H cleavage TS in the analyzed ensembles for structures within 5 kcal/mol (corresponding to >99.9% of Boltzmann population).

### 9. References

(1) Zong, Q.-S.; Wu, J.-Y. A New Approach to the Synthesis of Royal Jelly Acid. Chem. Nat. Compd. 2014, 50, 399-401.

(2) Cormanich, R. A.; Rittner, R.; Freitas, M. P.; Bühl, M. The seeming lack of CF··· HO intramolecular hydrogen bonds in linear aliphatic fluoroalcohols in solution. Phys. Chem. Chem. Phys. 2014, 16, 19212-19217.

(3) Olsen, E. P. K.; Madsen, R. Iridium-Catalyzed Dehydrogenative Decarbonylation of Primary Alcohols with the Liberation of Syngas. Chem. Eur. J. 2012, 18, 16023-16029.

(4) Fallek, A.; Weiss-Shtofman, M.; Kramer, M.; Dobrovetsky, R.; Portnoy, M. Phosphorylation Organocatalysts Highly Active by Design. Org. Lett. 2020, 22, 3722-3727.

(5) Zhu, Y.; Huang, K.; Pan, J.; Qiu, X.; Luo, X.; Qin, Q.; Wei, J.; Wen, X.; Zhang, L.; Jiao, N. Silver-catalyzed remote Csp³—H functionalization of aliphatic alcohols. Nature Communications 2018, 9, 2625.

(6) Komine, K.; Urayama, Y.; Hosaka, T.; Yamashita, Y.; Fukuda, H.; Hatakeyama, S.; Ishihara, J. Formal Synthesis of (−)-Haliclonin A: Stereoselective Construction of an Azabicyclo [3.3.1] nonane Ring System by a Tandem Radical Reaction. Org. Lett. 2020, 22, 5046-5050.

(7) Chen, F.; Song, K.-S.; Wu. Y.-D.; Yang, D. Synthesis and Conformational Studies of γ-Aminoxy Peptides. J. Am. Chem. Soc. 2008, 130, 743-755.

(8) Xu. G.-F.; Yang. X.-L.; Lei, P.; Liu, X.-L.; Zhang, X.-B.; Ling Y. Synthesis and fungicidal activity study of novel daphneolone analogs with 2,6-dimethylmorpholine. Chinese Chemical Letters, 2016, 27, 555-558.

(9) Yan, X.-C.; Harutyunyan, S. R. Double-slit photoelectron interference in strong-field ionization of the neon dimer. Nature Communications 2019, 10, 1.

(10) Park, H. S.; Fan, Z.-L.; Zhu, R.-Y.; Yu, J.-Q. Distal γ-C(sp³)—H Olefination of Ketone Derivatives and Free Carboxylic Acids. Angew. Chem. Int. Ed. 2020, 59, 12853-12859.

(11) García, J. M.; González, A.; Kardak, B. G.; Odriozola, J. M.; Oiarbide, M.; Razkin J.; Palomo, C. Copper-Catalyzed Enantioselective Conjugate Addition of Dialkylzinc Reagents to α′-Oxy Enones. Chem. Eur. J. 2008, 14, 8768-8771.

(12) You, C.; Li, S.; Li, X.; Lan, J.; Yang, Y.; Chung, L. W.; Lv, H.; Zhang, X. Design and Application of Hybrid Phosphorus Ligands for Enantioselective Rh-Catalyzed Anti-Markovnikov Hydroformylation of Unfunctionalized 1,1-Disubstituted Alkenes. J. Am. Chem. Soc. 2018, 140, 4977-4981.

(13) Ohno, H.; Okumura, M.; Maeda S.; Iwasaki H.; Wakayama, R.; Tanaka. T. Samarium (II)-Promoted Radical Spirocyclization onto an Aromatic Ring. J. Org. Chem. 2003, 68, 7722-7732.

(14) M. J. Frisch, G. W. Trucks, H. B. Schlegel, G. E. S.; M. A. Robb, J. R. Cheeseman, G. Scalmani, V. B.; G. A. Petersson, H. Nakatsuji, X. Li, M. Caricato, A. V. M.; J. Bloino, B. G. Janesko, R. Gomperts, B. Mennucci, H. P. H.; J. V. Ortiz, A. F. Izmaylov, J. L. Sonnenberg, D. W.-Y.; F. Ding, F. Lipparini, F. Egidi, J. Goings, B. Peng, A. P.; T. Henderson, D. Ranasinghe, V. G. Zakrzewski, J. Gao, N. R.; G. Zheng, W. Liang, M. Hada, M. Ehara, K. Toyota, R. F.; J. Hasegawa, M. Ishida, T. Nakajima, Y. Honda, O. Kitao, H. N.; T. Vreven, K. Throssell, J. A. Montgomery, Jr., J. E. P.; et al. Gaussian 16, Revision B.01. Gaussian, Inc.: Wallingford CT 2016.

(15) Becke, A. D. Density-Functional Thermochemistry. III. The Role of Exact Exchange. J. Chem. Phys. 1993, 98, 5648-5652.

(16) Lee, C.; Yang, W.; Parr, R. G. Development of the Colle-Salvetti Correlation-Energy Formula into a Functional of the Electron Density. Phys. Rev. B 1998, 37, 785-789.

(17) Becke, A. D. A New Mixing of Hartree-Fock and Local Density-Functional Theories. J. Chem. Phys. 1993, 98, 1372-1377.

(18) Stephens, P. J.; Devlin, F. J.; Chabalowski, C. F.; Frisch, M. J. Ab Initio Calculation of Vibrational Absorption and Circular Dichroism Spectra Using Density Functional Force Fields. J. Phys. Chem., 1994, 98, 11623-11627.

(19) Grimme, S.; Antony, J.; Ehrlich, S.; Krieg, H. A Consistent and Accurate Ab Initio Parametrization of Density Functional Dispersion Correction (DFT-D) for the 94 Elements H-Pu. J. Chem. Phys. 2010, 132, 154104.

(20) Grimme, S.; Ehrlich, S.; Goerigk, L. Effect of the Damping Function in Dispersion Corrected Density Functional Theory. J. Comput. Chem. 2011, 32, 1456-1465.

(21) (a) Martin, J. M. L.; Sundermann, A., Correlation consistent valence basis sets for use with the Stuttgart-Dresden-Bonn relativistic effective core potentials: The atoms Ga-Kr and In-Xe. J. Chem. Phys. 2001, 114, 3408-3420; (b) Andrae, D.; Häußermann, U.; Dolg, M.; Stoll, H.; Preuß, H., Energy-adjusted ab initio pseudopotentials for the second and third row transition elements. Theor. Chim. Acta 1990, 77, 123-141; (c) Dolg, M.; Wedig, U.; Stoll, H.; Preuss, H., Energy-adjusted ab initio pseudopotentials for the first row transition elements. J. Chem. Phys. 1987, 86, 866-872.

(22) Ditchfield, R.; Hehre, W. J.; Pople, J. A. Self-Consistent Molecular-Orbital Methods. IX. An Extended Gaussian-Type Basis for Molecular-Orbital Studies of Organic Molecules. J. Chem. Phys. 1971, 54, 720-723.

(23) Hehre, W. J.; Ditchfield, K.; Pople, J. A. Self-Consistent Molecular Orbital Methods. XII. Further Extensions of Gaussian-Type Basis Sets for Use in Molecular Orbital Studies of Organic Molecules. J. Chem. Phys. 1972, 56, 2257-2261.

(24) Hariharan, P. C.; Pople, J. A. The Influence of Polarization Functions on Molecular Orbital Hydrogenation Energies. Theor. Chim. Acta, 1973, 28, 213-222.

(25) Adamo, C.; Barone, V. Toward reliable density functional methods without adjustable parameters: The PBEO model. J. Chem. Phys. 1999, 110, 6158-6170.

(26) M Hong, D. P.; Hoshino, M.; Kuboi, R.; Goto, Y. Clustering of Fluorine-Substituted Alcohols as a Factor Responsible for Their Marked Effects on Proteins and Peptides. J. Am. Chem. Soc. 1999, 121, 8427-8433.

(27) Wohlfarth C, Wohlfarth B, Lechner MD. Optical constants. Springer; 1996.

(28) (a) Cances, E.; Mennucci, B.; Tomasi, J. J. Chem. Phys. 1997, 107, 3032-3041.; (b) Mennucci, B.; Tomasi, J. J. Chem. Phys. 1997, 106, 5151-5158.; (c) Scalmani, G.; Frisch, M. J. J. Chem. Phys. 2010, 132, 114110-114124.

(29) Grimme, S. Supramolecular Binding Thermodynamics by Dispersion-Corrected Density Functional Theory. Chem.-A Eur. J. 2012, 18, 9955-9964.

(30) Luchini, G.; Alegre-Requena, J. V; Funes-Ardoiz, I.; Paton, R. S. GoodVibes: Automated Thermochemistry for Heterogeneous Computational Chemistry Data. F1000Research 2020, 9, 291.

(31) Legault, C. Y. CYLView, 1.0b. Université de Sherbrooke 2009.

The foregoing disclosure has been described in some detail by way of illustration and example, for purposes of clarity and understanding. It will be obvious to one of skill in the art that changes and modifications may be practiced within the scope of the appended claims. Therefore, it is to be understood that the above description is intended to be illustrative and not restrictive. The scope of the disclosure should, therefore, be determined not with reference to the above description, but should instead be determined with reference to the following appended claims, along with the full scope of equivalents to which such claims are entitled.

This application refers to various publications (e.g., issued patents, published patent applications, journal articles, and other publications), each of which are incorporated herein by reference.

What is claimed is:

1. A process for preparing a compound of Formula (I) 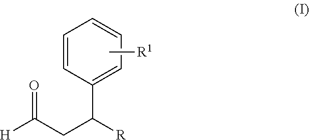
wherein:
R is C₁-C₁₀ alkyl that is optionally substituted with one substituent selected from the group consisting of C₃-C₇ cycloalkyl, C₆-C₁₀ aryl, halo, —O—C(═O)-(C₁-C₆ alkyl), —O-(C₆-C₁₀ aryl), and —O-(C₁-C₆ alkyl) wherein the C₁-C₆ alkyl of the —O-(C₁-C₆ alkyl) is optionally substituted with C₆-C₁₀ aryl; C₃-C₇ cycloalkyl; and C₆-C₁₀ aryl;
R¹ is hydrogen, halo, —C(═O)-(C₁-C₆ alkyl), halo(C₁-C₆ alkyl), C₁-C₆ alkyl, —NO₂, —C(═O)—OH, —CN, —C(═O)—O-(C₁-C₆ alkyl), and —O-(C₁-C₆ alkyl), or 13 O-halo(C₁-C₆ alkyl); comprising reacting a compound of Formula (II) 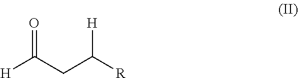
wherein R in Formula (II) is as defined above for Formula (I);
with a compound of Formula (III) 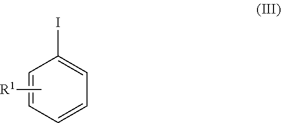
wherein R1 in Formula (III) is as defined above for Formula (I);
in the presence of a palladium salt, an amino acid based transient directing group compound (TDG), a 2-pyridone ligand (L), a salt composition comprising at least one silver salt, and an acid composition comprising acetic acid or an acetic acid derivative.

2. The process according to claim 1, wherein in R:
the optionally substituted C₁-C₁₀ alkyl is selected from the group consisting of —(CH₂)-n-C₆H₁₃, —CH₂CH₃, —CH₂CH₂CH₃, —CH₂-cyclohexyl, —CH₂CH₂-phenyl, —(CH₂)₄-CH₂F, —(CH₂)₂CH₂-naphthyl, —(CH₂)₂CH₂—O—C(—O)—CH₃, —(CH₂)₃CH₂—O-phenyl, —(CH₂)₄CH₂—O-n-C₃H₇, —(CH₂)₄CH₂—O—CH(CH₃)₂, —(CH₂)₄CH₂—O-benzyl, and —(CH₂)₄CH₂—O-naphthyl;
the C₃-C₇ cycloalkyl is cyclohexyl; and
the C₆-C₁₀ aryl is phenyl. (both inventors)

3. The process according to claim 1 or 2, wherein in R¹:
the —C(═O)—(C₁-C₆ alkyl) is —C(═O)—CH₃;
the halo(C₁-C₆ alkyl) is CF₃;
the —C(═O)—O—(C₁-C₆ alkyl) is —C(═O)—OCH₃;
the —O-(C₁-C₆ alkyl) is —OCH₃;l and
the —O-halo(C₁-C₆ alkyl) is —OCF₃.

4. The process according to any one of claims 1-3, wherein the compound of Formula (I) is selected from the group consisting of:

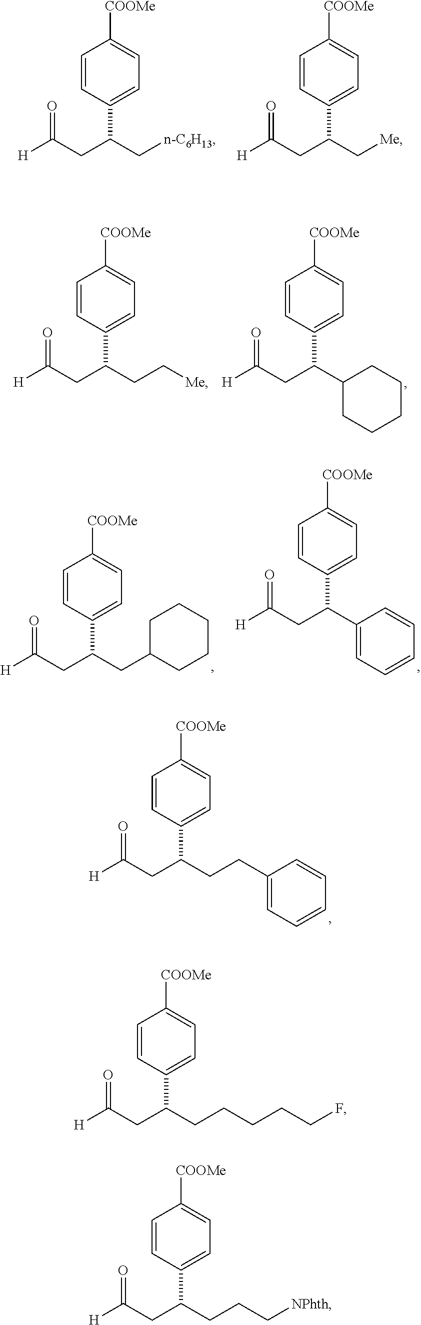

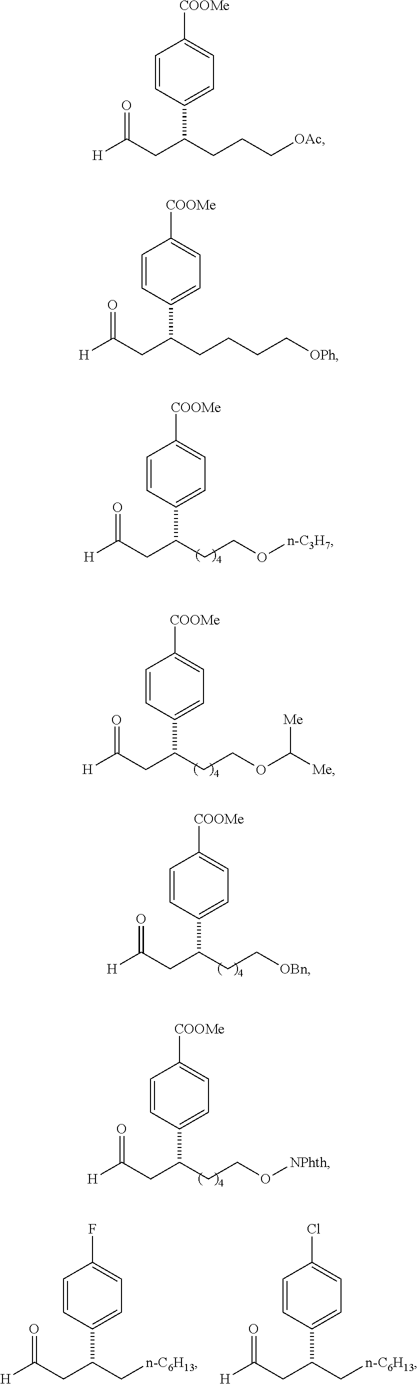

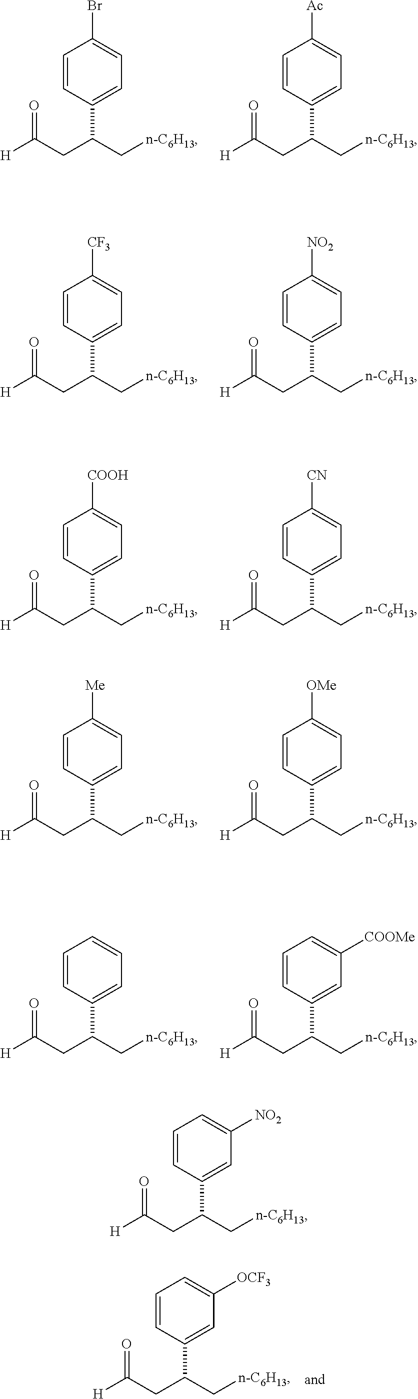

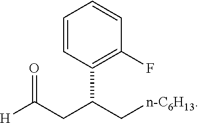

5. The process according to any one of claims 1-4, wherein the palladium salt is Pd(OAc)₂ Pd(TFA)₂, or PdCl₂.

6. The process according to any one of claims 1-5, wherein the TDG is selected from the group consisting of

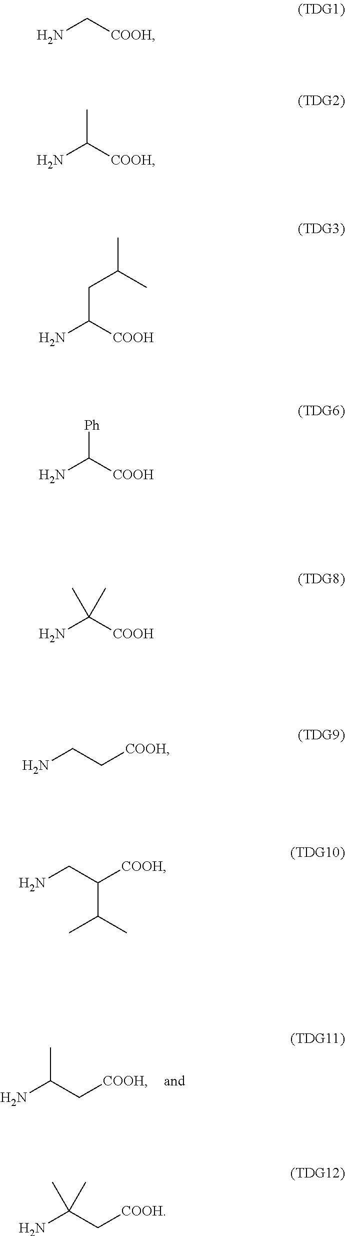

7. The process according to claim 6, wherein the TDG is TDG12.

8. The process according to any one of claims 1-7, wherein L is selected from the group consisting of

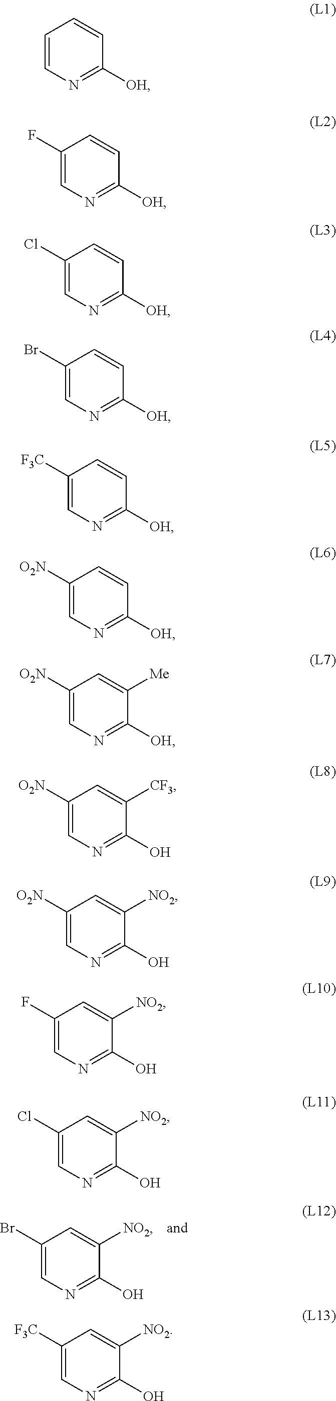

9. The process according to claim 8, wherein L is L8.

10. The process according to any one of claims 1-9, wherein the at least one silver salt of the salt composition is selected from the group consisting of silver trifluoroacetate (AgTFA), silver carbonate (Ag₂CO₃), silver acetate (AgOAc), and silver nitrate (AgNO₃).

11. The process according to claim 10, wherein the salt composition further comprises in addition to the silver salt, a metal salt distinct from the silver salt.

12. The process according to claim 11, wherein the metal salt distinct from the silver salt is selected from the group consisting of sodium carbonate (Na₂CO₃), lithium carbonate (Li₂CO₃), and cesium carbonate (Cs₂CO₃)

13. The process according to any one of claims 10-12, wherein the salt composition is selected from the group consisting of: a mixture of AgTFA and Ag₂CO_{3,} a mixture of AgTFA and Ag₂O, a mixture of AgTFA and Li₂CO₃, and a mixture of AgTFA and AgNO₃.

14. The process according to any one of claims 1-13, wherein the acetic acid derivative of the acid composition is selected from the group consisting of chloroacetic acid (ClCH₂COOH), trichloroacetic acid (Cl₃CCOOH), difluoroacetic acid (F₂CHCOOH), and trifluoroacetic acid (F₃CCOOH).

15. A process for preparing a compound of Formula (IV) 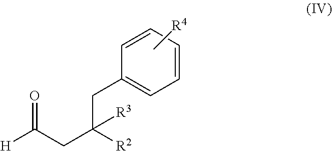
wherein:
R² is H, or C₁-C₆ alkyl;
R³ is C₃-C₇ cycloalkyl; or C₁-C₆ alkyl which is optionally substituted with a substituent selected from the group consisting of —O—C(═O)-C₁-C₆ alkyl and C₆-C₁₀ aryl; and
R⁴ is selected from the group consisting of —C(═O)-(C₁-C₆ alkyl); —C(═O)—O-(C₁-C₆ alkyl); —C(═O)—O -(C₃-C₁₀ bicyclic carbocyclyl) wherein said C₃-C₁₀ bicyclic carbocyclyl is optionally substituted with C₁-C₆ alkyl; halo; halo (C₁-C₆ alkyl); —NO₂; —CN; 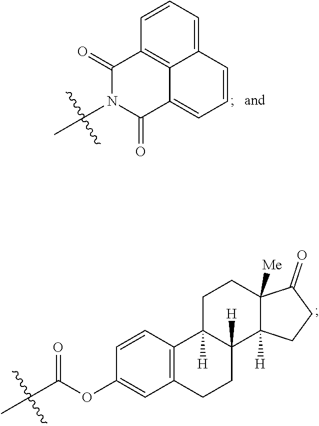
comprising reacting a compound of Formula (V) 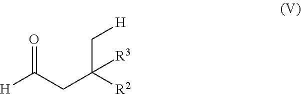
wherein R² and R³ in Formula (V) are as defined above for Formula (IV);
with a compound of Formula (VI) 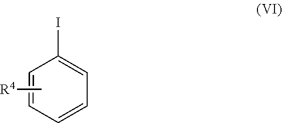
wherein R⁴ in Formula (VI) is as defined above for Formula (IV);
in the presence of a palladium salt, an amino acid based transient directing group compound (TDG), a 2-pyridone ligand (L), a salt composition comprising at least one silver salt, and an acid composition comprising acetic acid or an acetic acid derivative.

16. The process according to claim 15, wherein:
in R², the C₁-C₆ alkyl is methyl;
in R³, the C₃-C₇ cycloalkyl is cyclopentyl or cyclohexyl; the optionally substituted C₁-C₆ alkyl is —CH₃, —(CH₂)₄CH₃, —CH₂-t-Butyl, —(CH₂)₃CH(CH₃)₂, —(CH₂)₂—O—C(═O)—CH₃, —(CH₂)₂-phenyl, or —(CH₂)₃CH₃; and
in R⁴, the —C(═O)-(C₁-C₆ alkyl) is —C(═O)—CH₃; the —C(═O)—O-(C₁-C₆ alkyl) is —C(═O)—O—CH₃; the —C(═O)—O-(C₃-C₁₀ bicyclic carbocyclyl) with the optionally substituted bicyclic carbocyclyl is 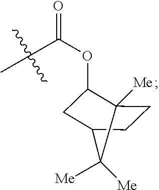 [SMILES: *C(=O)OC1CC2CCC1(C)C2(C)C]
and the halo (C₁-C₆ alkyl) is CF₃.

17. The process according to claim 15 or 16, wherein the compound of Formula (IV) is selected from the group consisting of:

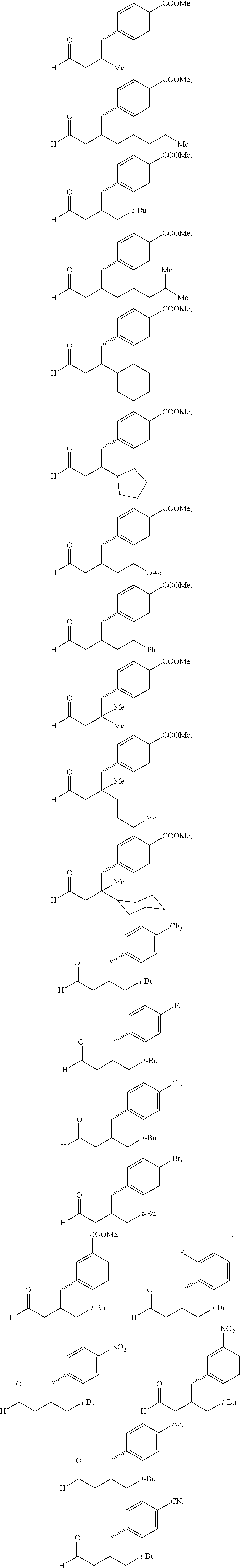

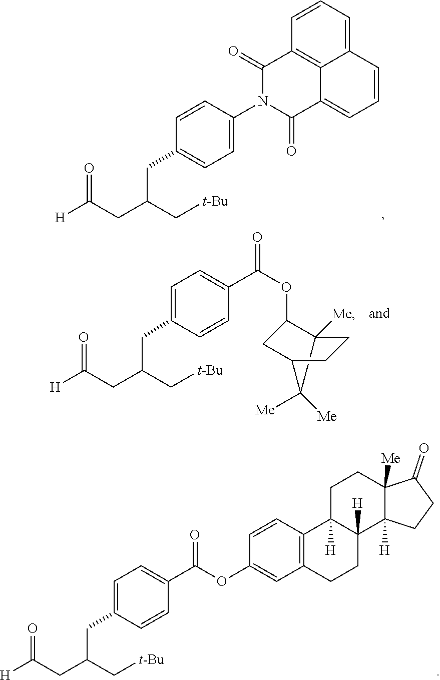

18. The process according to any one of claims 15-17, wherein the palladium salt is Pd(OAc)₂, Pd(TFA)₂, or PdCl₂.

19. The process according to any one of claims 15-18, wherein the TDG is selected from the group consisting of

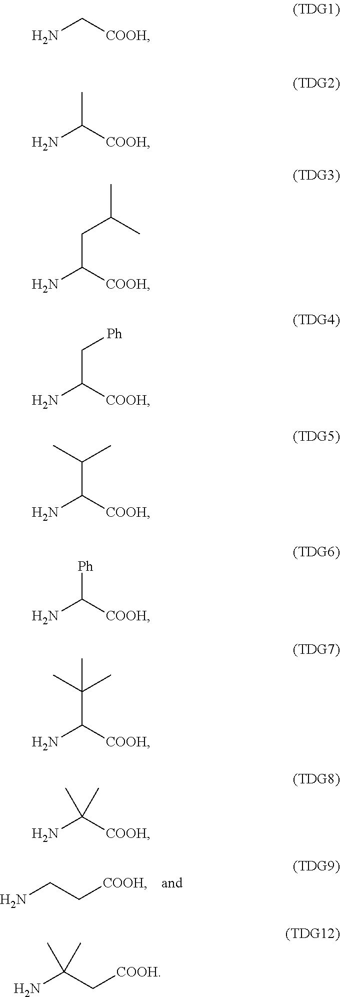

20. The process according to claim 19, wherein the TDG is TDG7.

21. The process according to any one of claims 15-20, wherein L is selected from the group consisting of

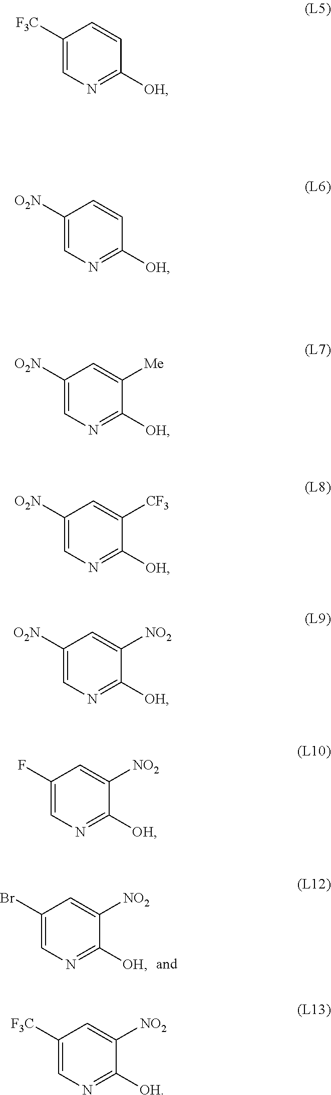

22. The process according to claim 21, wherein L is L8.

23. The process according to any one of claims 15-22, wherein the at least one silver salt of the salt composition is selected from the group consisting of silver trifluoroacetate (AgTFA), silver carbonate (Ag₂CO₃), silver acetate (AgOAc), and silver nitrate (AgNO₃).

24. The process according to claim 15, wherein the salt composition further comprises in addition to the silver salt, a metal salt distinct from the silver salt.

25. The process according to claim 24, wherein the metal salt distinct from the silver salt is selected from the group consisting of sodium carbonate (Na₂CO₃), lithium carbonate (Li₂CO₃), and cesium carbonate (Cs₂CO₃).

26. The process according to any one of claims 23-25, wherein the salt composition is selected from the group consisting of: a mixture of AgTFA and Ag₂CO₃, a mixture of AgTFA and Ag₂O, a mixture of AgTFA and Li₂CO₃, and a mixture of AgTFA and AgNO₃.

27. The process according to any one of claims 15-26, wherein the acetic acid derivative of the acid composition is selected from the group consisting of chloroacetic acid (ClCH₂COOH), trichloroacetic acid (Cl₃CCOOH), difluoroacetic acid (F₂CHCOOH), and trifluoroacetic acid (F₃CCOOH).

28. A process for preparing a compound of Formula (VII) 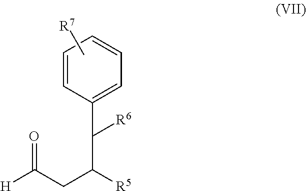
wherein:
R⁵ and R⁶ together with the carbon atoms to which they are shown attached form a C₄-C₇ cycloalkyl; and
R⁷ is selected from the group consisting of —C(═O)-(C₁-C₆ alkyl); —C(═O)—O-(C₁-C₆ alkyl); —C(═O)—O-(C₃-C₁₀ bicyclic carbocyclyl) wherein said C₃-C₁₀ bicyclic carbocyclyl is optionally substituted with C₁-C₆ alkyl; halo; halo (C₁-C₆ alkyl); —NO₂; —CN; 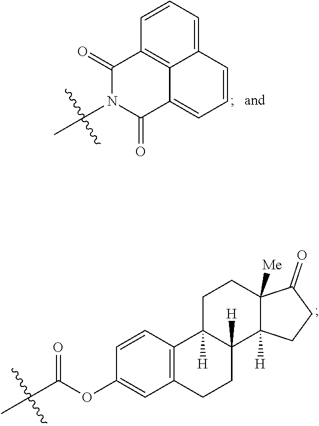
comprising reacting a compound of Formula (VIII) 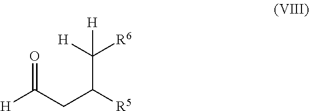
wherein R⁵ and R⁶ are as defined above for Formula (VII);
with a compound of Formula (IX) 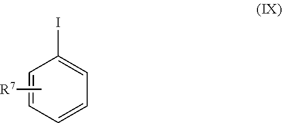
wherein R⁷ is as defined above for Formula (VII);
in the presence of a palladium salt, an amino acid based transient directing group compound (TDG), a 2-pyridone ligand (L), a salt composition comprising at least one silver salt, and an acid composition comprising acetic acid or an acetic acid derivative.

29. The process according to claim 28, wherein:
R⁵ and R⁶ together with the carbon atoms to which they are shown attached form a cyclohexyl group; and
in R⁷, the —C(═O)-(C₁-C₆ alkyl) is —C(═O)—CH₃; the —C(═O)—O-(C₁-C₆ alkyl) is —C(═O)—O—CH₃; the —C(═O)—O-(C₃-C₁₀ bicyclic carbocyclyl) with the optionally substituted bicyclic carbocyclyl is 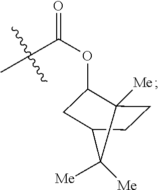 [SMILES: *C(=O)OC1CC2CCC1(C)C2(C)C]
and the halo (C₁-C₆ alkyl) is CF₃.

30. The process according to claim 29, wherein R⁷ is —C(═O)—O—CH₃.

31. The process according to any one of claims 28-30, wherein the compound of Formula (VII) is

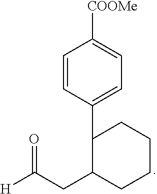 [SMILES: COC(=O)c1ccc(C2CCCCC2CC=O)cc1]

32. The process according to any one of claims 28-31, wherein the palladium salt is Pd(OAc)₂, Pd(TFA)₂, or PdCl₂.

33. The process according to any one of claims 28-32, wherein the TDG is selected from the group consisting of

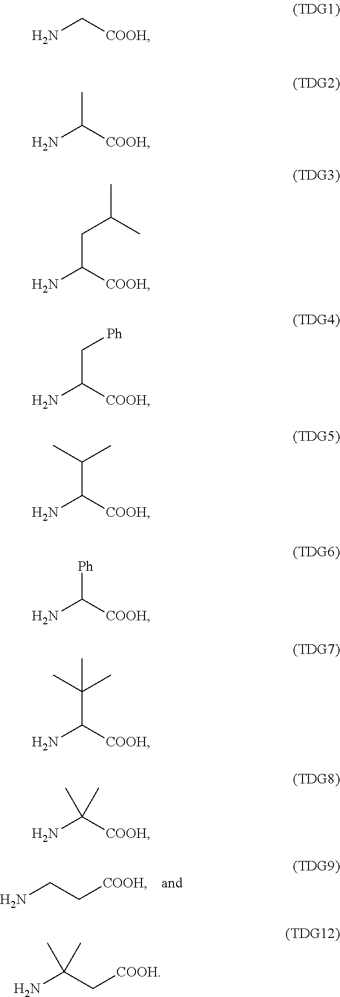

34. The process according to claim 33, wherein the TDG is TDG7.

35. The process according to any one of claims 28-34, wherein L is selected from the group consisting of

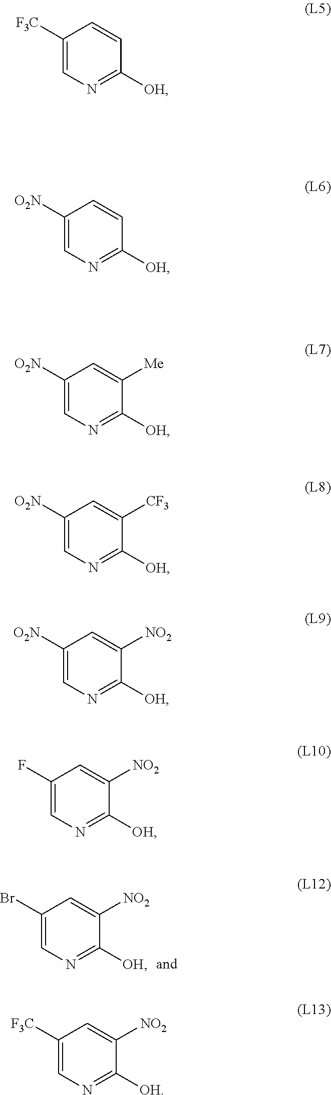

36. The process according to claim 35, wherein L is L8.

37. The process according to any one of claims 28-36, wherein the at least one silver salt of the salt composition is selected from the group consisting of silver trifluoroacetate (AgTFA), silver carbonate (Ag₂CO₃), silver acetate (AgOAc), and silver nitrate (AgNO₃).

38. The process according to claim 37, wherein the salt composition further comprises in addition to the silver salt, a metal salt distinct from the silver salt.

39. The process according to claim 38, wherein the metal salt distinct from the silver salt is selected from the group consisting of sodium carbonate (Na₂CO₃), lithium carbonate (Li₂CO₃), and cesium carbonate (Cs₂CO₃)

40. The process according to any one of claims 37-39, wherein the salt composition is selected from the group consisting of: a mixture of AgTFA and Ag₂CO₃, a mixture of AgTFA and Ag₂O, a mixture of AgTFA and Li₂CO₃, and a mixture of AgTFA and AgNO₃.

41. The process according to any one of claims 28-40, wherein the acetic acid derivative of the acid composition is selected from the group consisting of chloroacetic acid (ClCH₂COOH), trichloroacetic acid (Cl₃CCOOH), difluoroacetic acid (F₂CHCOOH), and trifluoroacetic acid (F₃CCOOH).
